(19) United States
(12) Patent Application Publication
(10) Pub. No.: US 2016/0102365 A1
(43) Pub. Date: Apr. 14, 2016

(54) ASSAYS, METHODS AND KITS FOR ANALYZING SENSITIVITY AND RESISTANCE TO ANTI-CANCER DRUGS, PREDICTING A CANCER PATIENT'S PROGNOSIS, AND PERSONALIZED TREATMENT STRATEGIES

(71) Applicant: UNIVERSITY OF MIAMI, Miami, FL (US)

(72) Inventor: Tan A. Ince, Miami, FL (US)

(21) Appl. No.: 14/894,595

(22) PCT Filed: Jun. 4, 2014
(86) PCT No.: PCT/US14/40806

§ 371 Date: Nov. 30, 2015

Related U.S. Application Data
(60) Provisional application No. 61/830,709, filed on Jun. 4, 2013.

(51) Int. Cl. C12Q 1/68; A61K 33/24; A61K 31/475; A61K 31/437; A61K 31/473; A61K 31/704; A61K 31/44; A61K 31/513; A61K 31/337; A61K 31/277

(52) U.S. Cl. CPC C12Q 1/6886; A61K 31/337; A61K 33/24; A61K 31/475; A61K 31/277; A61K 31/473; A61K 31/704; A61K 31/44; A61K 31/513; A61K 31/437; C12Q 2600/106; C12Q 2600/136; C12Q 2600/158

(57) ABSTRACT

Described herein are assays, methods and kits for analyzing sensitivity of a subject's cancerous tumor to a drug, predicting responses of cancerous tumors to drugs, determining the prognosis of a subject having a cancerous tumor, and developing a personalized therapy or treatment strategy for the subject. The assays, methods and kits involve analyzing gene and protein expression signatures or profiles of a subject's cancerous tumor, testing candidate drugs in cancerous cells from the subject's cancerous tumor, and classifying a subject's cancerous tumor based on ovarian cell and fallopian tube cell cell-of-origin gene expression signatures. Using these methods, a suitable drug (or drugs) is identified, the subject can be treated with that drug, and a personalized therapy can be developed for the subject.

### CROSS-REFERENCE TO RELATED APPLICATION

This application claims the benefit of Provisional Application Ser. No. 61/830,709 filed Jun. 4, 2013, which is herein incorporated by reference in its entirety.

### FIELD OF THE INVENTION

The invention relates generally to the fields of cellular biology, molecular biology, oncology, and medicine.

### BACKGROUND

Despite many decades of incremental improvements in methods for establishment of cancer cell lines, it is still extremely difficult to establish high-quality, permanent cell lines from human primary tumors routinely. Malignant, drug-resistant cancer phenotypes are not represented in currently available tumor cell line panels which fail to represent the biological diversity of human tumors. The inability to establish stable cell lines from the vast majority of human tumors has limited the use of in vitro models to study human cancer. A robust and efficient model system that predicts a patient's response to various drugs would greatly improve development of new drugs for personalized treatment of cancer patients. There is thus a need for the development of methods for testing and predicting a patient's response to treatment.

### SUMMARY

Assays, methods and kits for analyzing sensitivity of a subject's (e.g., patient's) cancerous tumor to a drug, predicting responses of cancerous tumors to drugs, determining the prognosis of a subject having a cancerous tumor, and developing a personalized therapy or treatment strategy for the subject are described herein. Identification of patients who are resistant to particular oncology drugs (e.g., Taxol) and in vitro determination of specific existing and new drugs to be utilized for individual patients can be achieved using the assays and methods described herein, providing for the development of a personalized approach to cancer treatment. Such assays include high throughput screening assays (e.g., high throughput screening of a group, plurality or population of patients or subjects and drugs).

Accordingly, described herein is a method for analyzing sensitivity of a subject's cancerous tumor to an oncology drug (e.g., including but not limited to Taxol, vincristine, U0126, PJ34, adriamycin, AS703026, 5-Fluorouracil, cisplatin, PLX4720, etc.) and developing a personalized therapy for the subject (e.g., a female human having an ovarian cancer tumor). The method includes the steps of: (a) obtaining cancer cells from the subject's cancerous tumor; (b) examining expression of a set of proteins or mRNAs in the cancerous cells, wherein overexpression or underexpression of the set of proteins or mRNAs relative to a control is associated with resistance to the oncology drug; and (c) correlating overexpression or underexpression of the set of proteins or mRNAs relative to the control with resistance of the subject's cancerous tumor to the oncology drug and correlating normal expression of the set of proteins or mRNAs relative to the control with sensitivity of the subject's cancerous tumor to the oncology drug. In an embodiment in which the set of proteins or mRNAs are overexpressed or underexpressed in the subject's cancerous tumor relative to the control, the method can further include administering to the subject an oncology drug (e.g., Taxol, vincristine, U0126, PJ34, adriamycin, AS703026, 5-Fluorouracil, cisplatin, and PLX4720, etc.) different from the oncology drug the subject's cancerous tumor is resistant to. In an embodiment in which the set of proteins or mRNAs are normally expressed relative to the control, the method can further include administering the oncology drug to the subject. In one embodiment, the oncology drug is Taxol or vincristine, and the set of proteins includes at least two of: tubulin, AKT, androgen receptor, Jun oncogene, Crystalline, cyclin D1, epidermal fatty acid binding protein, Ets related gene, FAK, Forkhead Box O3, Erk/Mek, N-cadherin, mitogen-activated protein kinase 14, plasminogen activator inhibitor type 1, paired box 2, protein kinase C-alpha, protein kinase AMP-activated Gamma 2, phosphatase and tensin homolog, SMAD3, Sarcoma viral oncogene homolog, signal transducer and activator of transcription 3, and signal transducer and activator of transcription 5.

The method can further include correlating overexpression or underexpression of the set of proteins or mRNAs relative to the control with a worse prognosis for the subject compared to a second subject having a cancerous tumor in which the first set of proteins or mRNAs are normally expressed relative to the control. The method can further include repeating steps b) and c) until an oncology drug that the subject's cancerous tumor is sensitive to is identified.

Also described herein is a method for predicting a response of a cancer patient's (e.g., a female human having an ovarian cancer tumor) cancerous tumor to an oncology drug (e.g., Taxol, vincristine, U0126, PJ34, adriamycin, AS703026, 5-Fluorouracil, cisplatin, PLX4720, etc.) and developing a personalized therapy for the patient for treatment of the cancerous tumor. The method includes the steps of: obtaining cancer cells from the patient's cancerous tumor; culturing the cancer cells in WIT-OC, WIT-L, or WIT-OCe cell culture medium; contacting the cultured cancer cells with the oncology drug; determining an IC50 OR IC90 value for the oncology drug in the cultured cancer cells; and correlating an increased IC50 or IC90 value relative to an IC50 or IC90 value for the oncology drug in control cultured cells with a poor response of the patient's cancerous tumor to the oncology drug and correlating a normal or low IC50 or IC90 value relative to the IC50 or IC90 value for the oncology drug in control cultured cells with a positive response of the patient's cancerous tumor to the oncology drug. The cancer cells can be, for example, ovarian cancer cells obtained from ascites fluid or primary solid ovarian tissue from the patient. In one embodiment, the IC50 or IC90 value is increased relative to the IC50 or IC90 value for the oncology drug in control cultured cells, and the method further includes administering to the patient a second oncology drug (e.g., Taxol, vincristine, U0126, PJ34, adriamycin, AS703026, 5-Fluorouracil, cisplatin, PLX4720, etc.). In another embodiment, the IC50 or IC90 value is normal or decreased relative to the IC50 or IC90 value for the oncology drug in control cultured cells, and the method further includes administering the oncology drug to the patient. The method can further include correlating an increased IC50 or IC90 value relative to an IC50 or IC90 value for the oncology drug in control cultured cells with a worse prognosis for the patient compared to a second patient having a cancerous tumor in which an IC50 or IC90 value for the oncology drug in cultured cancer cells from the second patient is normal or decreased relative to the IC50 or IC90 value for the oncology drug in control cultured cells.

Still further described herein is a kit for analyzing sensitivity of a subject's cancerous tumor and predicting a response of a subject's (e.g., cancer patient's) cancerous tumor to an oncology drug and developing a personalized therapy for the subject. The kit includes one or more OCI lines as an internal control(s); instructions for use; WIT medium, or a derivative of WIT medium; and optionally, one or more probes. In such a kit, the one or more probes can be probes specific to at least two (e.g., two, three, four, five, etc.) of the following proteins: tubulin, AKT, androgen receptor, Jun oncogene, Crystalline, cyclin D1, epidermal fatty acid binding protein, Ets related gene, FAK, Forkhead Box O3, Erk/Mek, N-cadherin, mitogen-activated protein kinase 14, plasminogen activator inhibitor type 1, paired box 2, protein kinase C-alpha, protein kinase AMP-activated Gamma 2, phosphatase and tensin homolog, SMAD3, Sarcoma viral oncogene homolog, signal transducer and activator of transcription 3, and signal transducer and activator of transcription 5.

Additionally described herein is a method for determining a prognosis of a subject (e.g., a female human) having an ovarian cancer tumor. The method includes the steps of: obtaining a sample from the subject's tumor; subjecting the sample to gene expression profiling resulting in an expression profile comprising a first set of genes that are upregulated in fallopian tube cells relative to ovarian cells and a second set of genes that are upregulated in ovarian cells relative to fallopian tube cells; determining expression levels of the first and second sets of genes; and correlating an upregulation of the first set of genes but not of the second set of genes with a worse disease-free survival prognosis relative to a second subject having an ovarian cancer tumor in which the first set of genes are not upregulated and the second set of genes are upregulated. In one embodiment, the first set of genes includes DOK5, CD47, HS6ST3, DPP6, and OSBPL3 and the second set of genes includes STC2, SFRP1, SLC35F3, SHMT2, and TMEM164. In an embodiment in which the first set of genes in the expression profile is upregulated, the method can further include classifying the subject's ovarian cancer tumor as fallopian tube-like. In an embodiment in which the second set of genes in the expression profile is upregulated, the method can further include classifying the subject's ovarian cancer tumor as ovary-like. The method can further include administering an oncology drug to the subject.

Unless otherwise defined, all technical terms used herein have the same meaning as commonly understood by one of ordinary skill in the art to which this invention belongs.

As used herein, “protein” and “polypeptide” are used synonymously to mean any peptide-linked chain of amino acids, regardless of length or post-translational modification, e.g., glycosylation or phosphorylation.

By the term “gene” is meant a nucleic acid molecule that codes for a particular protein, or in certain cases, a functional or structural RNA molecule.

As used herein, a “nucleic acid” or a “nucleic acid molecule” means a chain of two or more nucleotides such as RNA (ribonucleic acid) and DNA (deoxyribonucleic acid).

The terms “patient,” “subject” and “individual” are used interchangeably herein, and mean an animal (e.g., a mammal such as a human, a vertebrate) subject to be treated and/or to obtain a biological sample from.

When referring to a nucleic acid molecule or polypeptide, the term “native” refers to a naturally-occurring (e.g., a wild type, WT) nucleic acid or polypeptide.

As used herein, the phrases “WIT-OC cell culture medium,” “WIT-oc cell culture medium,” “WIT-OC medium” and “WIT-oc medium” are used interchangeably and refer to a cell culture medium adapted for the culture of tumor cells (such as ovarian tumor cells) and including between 1.0% and 10.0% v/v of serum (preferably between 1.8% v/v and 2% v/v of serum, most preferably about 1.8% v/v of serum). In some such embodiments, WIT-OC cell culture medium includes between 0.15 μg/mL and 0.3 μg/mL of hydrocortisone, preferably about 0.15 μg/mL of hydrocortisone and/or between 5.0 μg/mL and 50.0 μg/mL of insulin, preferably about 15.0 μg/mL of insulin. In such embodiments adapted for the culture of certain ovarian tumor cells, such as those derived from endometrioid tumors and mucinous tumors, WIT-OC cell culture medium further includes an estrogen, for example an estrogen (e.g., 17-beta-estradiol) at a concentration of equivalent potency of between 30 nM and 300 nM of 17-beta-estradiol, preferably about 100 nM of 17-beta-estradiol. In other embodiments, such as those adapted for the culture of certain ovarian tumor cells, such as tumor cells derived from papillary serous tumors, clear cell tumors, carcinosarcomas, and dysgerminomas, WIT-OC cell culture medium is substantially free of estrogens. WIT-OC cell culture medium may include estrogen or may be substantially free of estrogen, depending on the cell type that will be cultured therein. WIT-OC cell culture medium is described in detail in PCT application no. PCT/US2012/030446 and U.S. application Ser. No. 14/007,008, which are both incorporated herein by reference in their entireties.

The phrases “WIT-FO cell culture medium,” “WIT-fo cell culture medium,” “WIT-FO medium” and “WIT-fo medium” are used interchangeably herein to mean a modified version of WIT-OC cell culture medium optimized for fallopian tube and ovarian epithelial cells. WIT-fo medium was modified with several supplements to a final concentration of 0.5 to 1% serum, and supplemented with EGF (0.01 ug/mL, Sigma, E9644), Insulin (20 ug/mL, Sigma, 10516), Hydrocortisone (0.5 ug/mL, Sigma H0888) and 25 ng/mL Cholera Toxin (Calbiochem, 227035).

By the term “off label” when referring to a drug or compound means that the drug or compound is used in a different way than described in the FDA-approved drug or compound label.

As used herein, the terms “therapeutic,” and “therapeutic agent” are used interchangeably, and are meant to encompass any molecule, chemical entity, composition, drug, therapeutic agent, chemotherapeutic agent, or biological agent capable of preventing, ameliorating, or treating a disease or other medical condition. The term includes small molecule compounds, antisense reagents, siRNA reagents, antibodies, enzymes, peptides organic or inorganic molecules, cells, natural or synthetic compounds and the like.

The term “sample” is used herein in its broadest sense. A sample including polynucleotides, proteins, peptides, antibodies and the like may include a bodily fluid, a soluble fraction of a cell preparation or media in which cells were grown, genomic DNA, RNA or cDNA, a cell, a tissue, a biopsy, skin, hair and the like. Examples of samples include saliva, serum, tissue, biopsies, skin, blood, urine and plasma.

As used herein, the term “treatment” is defined as the application or administration of a therapeutic agent to a patient or subject, or application or administration of the therapeutic agent to an isolated tissue or cell line from a patient or subject, who has a disease, a symptom of disease or a predisposition toward a disease, with the purpose to cure, heal, alleviate, relieve, alter, remedy, ameliorate, improve, prevent or affect the disease, the symptoms of disease, or the predisposition toward disease.

Although assays, kits, and methods similar or equivalent to those described herein can be used in the practice or testing of the present invention, suitable assays, kits, and methods are described below. All publications, patent applications, and patents mentioned herein are incorporated by reference in their entirety. In the case of conflict, the present specification, including definitions, will control. The particular embodiments discussed below are illustrative only and not intended to be limiting.

### BRIEF DESCRIPTION OF THE DRAWINGS

FIG. 1: Gene expression profiling of ovarian cancer cell lines and ovarian tumor samples identifies two major classes. A) Unsupervised hierarchical clustering of gene expression data of 37 cell lines and 285 human tissues. Genes with an expression level that was at least 2-fold different relative to the median value across tissues in at least 4 cells were selected for hierarchical clustering analysis (3,831 gene features). The data are presented in matrix format in which rows represent individual genes and columns represent each tissue. Each square in the matrix represents the expression level of a gene feature in an individual tissue or cell line. The red and green color in cells reflect relative high and low expression levels respectively as indicated in the scale bar (log 2 transformed scale). Red, blue and black bars above the heatmap are human tumor samples; light blue, OCI lines; yellow bar SOC lines. Whereas SOC cells (yellow bars) were exclusively within tumor cluster 1 (red A bar), the OCI cells (light blue bars) were predominantly within tumor cluster 2 (blue bar). A small subset of tumor samples formed a small distinct cluster that did not include any cell lines (black bar). B) The progression-free and overall survival analysis data of patients with the ovarian tumors in clusters 1 and 2 in panel A. The patients with tumors that have a gene expression profile that is similar to OCI lines (blue bar, cluster 2 in panel A) have a worse outcome than patients with tumors that have gene expression profile similar to SOC lines (red bar, cluster 1 in panel A). The small subset of tumors in cluster 3 that did not include any cell lines (black bar) was excluded from the outcome analysis.

FIG. 2: Gene expression profiling and Taxol Response of ovarian cancer cell lines identifies two major classes. A) Taxol response of OCI and SOC cell lines in mRNA/RPPA (Reverse Phase Protein Analysis) Cluster 1 vs. Cluster 2. The OCI and SOC lines were plated in triplicates in WIT-OC medium (5000 cells/well) in 96 well plates. The next day 20 nM Taxol was added and metabolic activity was measured as 590/530 fluorescence via Alamar Blue after 5 days. OCI cell lines in mRNA/RPPA Cluster 1 (blue bars), SOC cell lines in Cluster 2 (red bars), OCI lines in Cluster 2 (white bars). The results are representative of more than three four different experiments. B) Proteins that are over-expressed in OCI mRNA/RPPA Cluster 1. The hierarchical clustering of RPPA data from OCI and SOC lines revealed a subset of proteins that are over-expressed in OCI lines significantly correlated with Taxol resistance (Cluster 1, blue labels; Cluster 2, red labels; OCI-C4p purple label, IC-50, p<0.05, Spearman). The data are presented in matrix format in which rows represent cell lines and columns represent antibody probes for each protein. The red and green colors reflect relative high and low expression levels respectively. C) Hierarchical clustering of mRNA data from OCI and SOC lines. The mRNA for the subset of genes associated with Taxol response in the RPPA analysis was examined. The mRNA clustering of the cell lines was very similar to RPPA groups. The over expressed genes are in red, under expressed genes are in green. A detailed list of genes that are up-regulated in each group is provided in Table 2 (list of proteins associated with Taxol resistance association that are over-expressed in Taxol resistant Cluster 1 OCI cells compared to Taxol sensitive class 2 cells in RPPA analysis). Cluster 1, blue labels; Cluster 2, red labels; OCI-C4p purple label.

FIG. 3: Gene expression profiling of ovarian cancer cell lines identifies two major classes. A) Unsupervised hierarchical clustering of mRNA expression levels of OCI (blue bars) and SOC (red bars) ovarian cancer cell lines. The data are presented in matrix format in which rows represent individual genes and columns represent each cell line. Each square in the matrix represents the expression level of a gene feature in an individual tissue or cell line. The red and green color in cells reflect relative high and low expression levels respectively as indicated in the scale bar (log 2 transformed scale). Two major clusters are observed; Cluster I contains only OCI cell lines (left cluster, blue only), and Cluster II contains a mixture of SOC and OCI cell lines (right cluster, red and blue). Interestingly, while the papillary serous histotype almost exclusively aligned within Cluster I (green bars), the other subtypes were present in both clusters (orange bars). B) The dendogram of the cell lines that make up the two clusters in the heatmap in panel A. The cell line names are colored as follows; first column OCI (blue), SOC (red); second column Papillary Serous (dark green), other histotypes (orange); third column Papillary Serous (dark green), Clear Cell (light blue), Endometrioid (pink), mucinous (light green), other histotypes (orange).

FIG. 4: The proteomic profile of ovarian cancer cell lines identifies two major classes. A) The unsupervised clustering of protein expression (measured by RPPA) in OCI cell lines (blue bars) together with SOC ovarian cancer cell lines (red bars) revealed two distinct clusters. Rows represent cell lines and columns represent antibody probes for each protein. The red and green colors reflect relative high and low expression levels, respectively. As in the mRNA clustering, Cluster 1 contains only OCI cell lines (top half of the heatmap, blue only), and Cluster 2 contains a mixture of SOC and OCI cell lines (bottom half of the heatmap, red and blue). While the papillary serous histotype almost exclusively aligned within Cluster 1 (green bars), the non-papillary serous subtypes (orange bars) were divided between Cluster 1 and Cluster 2. B) The dendogram of the cell lines that make up the two clusters in panel A. The cell line names are colored as follows; Papillary Serous (green), other histotypes (orange). See also FIG. 8.

FIG. 5: Histopathology of OCI xenografts recapitulates the original human tumor. A-C) H&E stained sections of primary human tumors used to create OCI-P8p (papillary serous), OCI-E1p (endometrioid) and OCI-C3x (clear cell). D-F) H&E stained sections of xenografts tumors derived by injecting SOC cells (ES2, SKOV3, and TOV-112D) subcutaneously into immunocompromised mice. The typical features of human adenocarcinomas such as glands, papillae, stromal cores, and desmoplastic stroma are absent. G-O) H&E stained sections of xenograft tumors derived by injecting OCI cell lines (P5x, P7a, P9a, C5x, C3x, CSp, E1p) subcutaneously into immunocopromised mice. In papillary serous specimens note the presence of stromal cores and papillary architecture (G, H and I). In the endometrioid specimen note the presence of glands (M), which were positive for estrogen receptor (ER) and mucin (brown), respectively, consistent with the endometrioid phenotype (N and O).

FIG. 6: The mRNA expression profiles of OCI cell lines in Cluster 1 and Cluster 2 are associated with distinct pathways. For pathway analysis we used Ingenuity Pathway Analysis (IPA) to organize the 823 genes that were significantly differentiate expressed between Cluster 1 vs. in Cluster 2 (p, 0.05) (FIG. 3). A) 558 were up-regulated in Cluster 1 which were organized in 37 core pathways in IPA (p<0.05). B) 265 genes were up-regulated in Cluster 2 which were organized in 37 core pathways in IPA (p<0.05).

FIG. 7: Validation of ten probesets associated with unique genes and over-expressed in either OCE or FNE in two independent ovarian cancer datasets. (a) Association of OV-like and FT-like tumor subclassification in the Wu dataset with clinical characteristics (P-values from logistic regression (grade, stage as ordinal variables) and Fisher's Exact test (histological subtype)). (b) Association of OV-like and FT-like subgroups in the Tothill dataset with clinical features (P-values calculated as in (a)). (c) Kaplan-Meier plots demonstrate significant differences in disease-free and overall survival between OV- and FT-like subgroups in the Tothill data (univariate P-values from the log-rank test are displayed). In multivariate analysis, the OV/FT-like subgroups were independently associated with disease-free survival (Cox proportional hazards P=0.01) but not overall survival (P=0.34) after adjusting for tumor grade, stage, serous subtype, patient age and residual disease.

FIG. 8: A series of graphs and a table showing that OCI lines are significantly more resistant to a diverse panel of oncology drugs compared to standard cell lines.

### DETAILED DESCRIPTION

Described herein are assays, methods and kits for analyzing sensitivity of a subject's cancerous tumor to a drug, predicting responses of cancerous tumors to drugs, determining the prognosis of a subject having a cancerous tumor, and developing a personalized therapy or treatment strategy for the subject. The assays, methods and kits involve analyzing gene and protein expression signatures or profiles of a subject's cancerous tumor, testing candidate drugs in cancerous cells from the subject's cancerous tumor, and classifying a subject's cancerous tumor based on ovarian cell and fallopian tube cell cell-of-origin gene expression signatures. Using these methods, a suitable drug (or drugs) is identified, the subject can be treated with that drug, and a personalized therapy is thus developed for the subject.

### Biological and Chemical Methods

Methods involving conventional molecular biology techniques are described herein. Such techniques are generally known in the art and are described in detail in methodology treatises such as Molecular Cloning: A Laboratory Manual, 3rd ed., vol. 1-3, ed. Sambrook et al., Cold Spring Harbor Laboratory Press, Cold Spring Harbor, N. Y., 2001; and Current Protocols in Molecular Biology, ed. Ausubel et al., Greene Publishing and Wiley-Interscience, New York, 1992 (with periodic updates). Conventional methods of culturing mammalian cells are generally known in the art. Methods of culturing ovarian and fallopian tube cells (e.g., ovarian cancer cells and fallopian tube cancer cells), including preparation and use of WIT-OC cell culture medium, are described in detail in PCT application no. PCT/US2012/030446. Any WIT culture medium or derivative of WIT culture medium (e.g., WIT-P, WIT-I, WIT-T, WIT-OC, WIT-OCe, WIT-L etc.) can be used.

### Methods and Assays for Analyzing Sensitivity of a Subject's Cancerous Tumor to a Drug and Developing a Personalized Therapy

Using the methods described herein, a prediction of a particular drug's (e.g., oncology drug) effect on a subject's cancerous tumor may be made, based on the expression profile of a particular set of proteins in the cancerous tumor, and a comparison to a control or reference cell line for which responsiveness to that particular drug is known. Generally, if a subject's cancerous tumor has a protein expression profile substantially similar to that of a control or reference cell line, and the control or reference cell line is responsive to treatment with a particular drug (e.g., oncology drug such as Taxol), then one can predict that the subject's cancerous tumor will also respond to treatment with that particular drug (e.g., Taxol). Conversely, if a subject's cancerous tumor has a protein expression profile substantially similar to that of a control or reference cell line, and the control or reference cell line is resistant to treatment with a particular drug (e.g., Taxol), then one can predict that the subject's cancerous tumor will also be resistant to treatment with that particular drug (e.g., Taxol).

A typical method for analyzing sensitivity of a subject's (e.g., mammal such as a human) cancerous tumor to a drug (e.g., Taxol) and developing a personalized therapy for the subject includes the steps of: obtaining cancer cells from the subject's cancerous tumor; examining expression of a set of proteins or mRNAs in the cancerous cells; and correlating overexpression or underexpression of the set of proteins or mRNAs relative to a control with resistance of the subject's cancerous tumor to the oncology drug and correlating normal expression of the set of proteins or mRNAs relative to the control with sensitivity of the subject's cancerous tumor to the oncology drug. The subject may be any animal, e.g., mammals such as human, bovine, canine, ovine, feline, non-human primate, porcine, etc. For example, the subject may be a female human having at least one (e.g., one, two, three, etc.) ovarian cancer tumor. The cancerous tumor may be any type of cancerous tumor. Examples of cancerous tumors include those from ovary, fallopian tube, lung, breast, colon, prostate, gastrointestinal, endocrine organ, blood, immune cell, muscle, bone, neural, endothelial, fibroblasts, or other epithelial and stromal tumors.

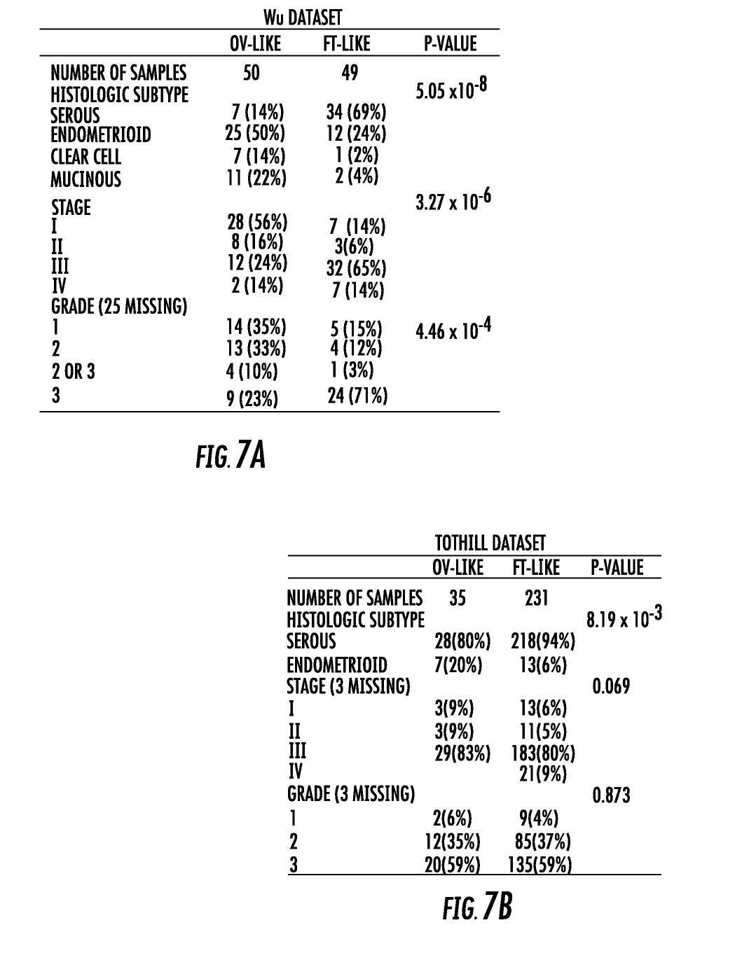

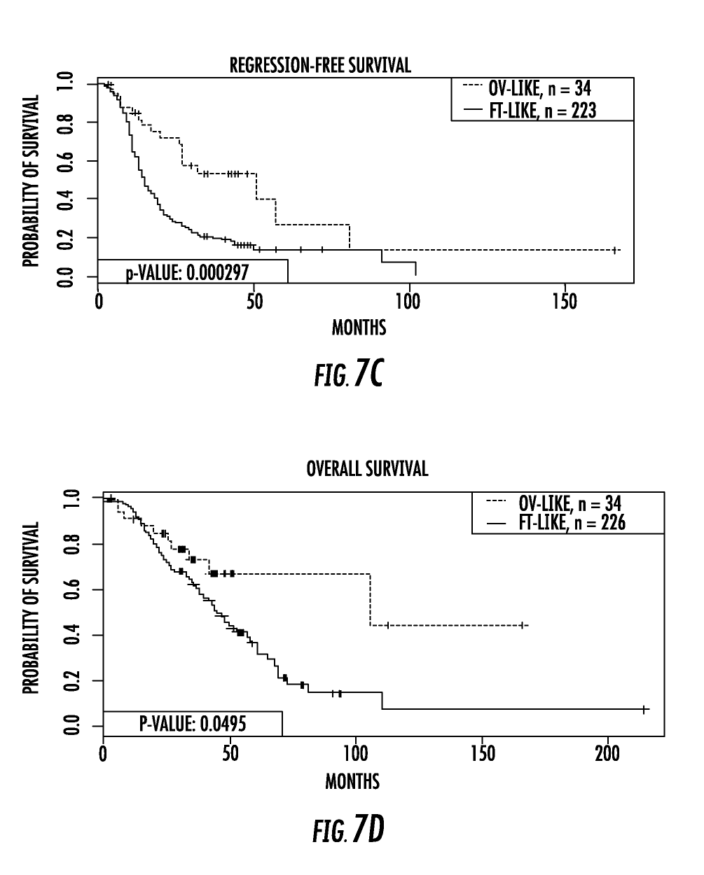

In this example of a method, the set of proteins or mRNAs includes proteins whose overexpression or underexpression relative to a control is associated with resistance to the drug. The set of proteins or mRNAs can include a subset of proteins or mRNAs whose overexpression is associated with resistance to the drug as well as a subset of proteins or mRNAs whose underexpression is associated with resistance to the drug. Typically, the drug is a known oncology drug. In one embodiment, the oncology drug is Taxol or vincristine, and the set of proteins includes at least two of: tubulin, AKT, androgen receptor, Jun oncogene, Crystalline, cyclin D1, epidermal fatty acid binding protein, Ets related gene, FAK, Forkhead Box O3, Erk/Mek, N-cadherin, mitogen-activated protein kinase 14, plasminogen activator inhibitor type 1, paired box 2, protein kinase C-alpha, protein kinase AMP-activated Gamma 2, phosphatase and tensin homolog, SMAD3, Sarcoma viral oncogene homolog, signal transducer and activator of transcription 3, and signal transducer and activator of transcription 5 (e.g., two or more (i.e., two, three, four, five, six, seven, eight, nine, ten, fifteen, twenty, etc.) of the proteins listed in Table 1 below). In the experiments described herein, the proteins listed in Table 1 were found to be overexpressed in OCI lines in the mRNA/RPPA Cluster 1 and associated with Taxol resistance in an RPPA analysis. Use of this method is not limited to Taxol, however. The same approach can be applied to any other oncology drug. As shown in FIG. 7, the methods described herein can be used for any oncology drug, e.g., Taxol, vincristine, U0126, PJ34, adriamycin, AS703026, 5-Fluorouracil, cisplatin, PLX4720, etc. Drugs that are considered off-label may also be analyzed using the methods.

Any suitable method of obtaining cancer cells from a subject's cancerous tumor can be used. In a typical method, cancer cells are obtained by a biopsy, needle aspirations, ascites fluid, or any other fluid containing tumor cells or solid tumor fragments removed during surgery. The cancer cells may be also obtained from a xenograft explant. In some embodiments, the method is used to simultaneously analyze the sensitivity of cancerous tumors from multiple subjects (e.g., 2, 3, 4, 5, 10, 15, 20, 25, 30, 35, 40, 50, 100, 1000, 10,000, etc.) who have cancer. In some embodiments, cancer cells from a plurality of subjects can be analyzed simultaneously, e.g., in a high-throughput format.

In the method, any suitable control sample can be used. Typically, the control sample is normal cells isolated from the same patient and same tissue, or cell lines established from other patients with a known drug response—sensitive or resistant, and expression of the set of proteins in the subject's cancerous cells is examined relative to expression levels of the set of proteins in this control sample. When referring to “overexpression” of the proteins in the set of proteins, what is meant is at least a two-fold increase compared to a control. Expression of a particular protein or set of proteins in a sample or population of cancerous cells can be compared to a baseline level (also known as a control level) of expression of the particular protein or set of proteins (e.g., a protein(s) listed in Table 1). A “baseline level” is a control level, and in some embodiments a normal level or a level not observed in subjects having cancer (e.g., ovarian cancer) or cell lines that are sensitive to a drug. Alternatively, a “baseline level” or control level is a level not observed in a sample from subjects having a different type of cancer (e.g., ovarian-like ovarian cancer) than the cancer (e.g., fallopian tube-like ovarian cancer) of the subject whose cancerous cells are being analyzed for sensitivity or resistance to an oncology drug. Therefore, it can be determined, based on the control or baseline level of expression of the particular protein (or set of proteins), whether a sample of cancer cells to be evaluated for sensitivity or resistance to a particular drug (e.g., Taxol) has a measurable increase (i.e., overexpression, upregulation), decrease, or substantially no change in expression of the particular protein (or set of proteins), as compared to the baseline level.

Expression of a set of proteins in the cancerous cells can be analyzed using any suitable techniques or protocols. For example, a Reverse Phase Protein Analysis (RPPA) assay (Zhang et al., Bioinformatics 25, 650-654, 2009) can be used. Conventional methods of analyzing protein expression include enzyme-linked detection systems such as enzyme-linked immunosorbent assays (ELISAs), fluorescence-based detection systems, Western blots, ELISAs, etc. In some embodiments, protein expression can be extrapolated by analyzing corresponding mRNA levels. Conventional methods of analyzing mRNA levels include reverse transcription polymerase chain reaction (RT-PCR), quantitative PCR, Serial analysis of gene expression (SAGE), RNA-Seq, next-generation sequencing, northern blotting, microarrays, etc.

In some embodiments, the steps of the method can be repeated for different oncology drugs until an oncology drug that the subject's cancerous tumor is sensitive (responsive) to is identified. If it turns out a patient's tumor is resistant to Taxol, the method can be repeated with a different set of proteins and another oncology drug(s) until an oncology drug the tumor will respond to is found. In some embodiments, after determining that a patient's tumor is resistant to Taxol, a second oncology drug may instead be administered to the patient without first testing resistance of the patient's tumor to the second oncology drug.

Once a suitable drug (or drugs) is identified, the subject can be treated with that drug, and a personalized therapy can be developed for the subject. More specifically, a treatment can be selected for the subject based at least in part on a prediction or result suggesting that a particular oncology drug will be effective or more effective than one or more alternative oncology drugs for that particular subject. For example, if the set of proteins or mRNAs are overexpressed or underexpressed in the subject's cancerous tumor relative to the control sample, it is determined that the subject's cancerous tumor is not sensitive to (i.e., is resistant to) the first oncology drug (e.g. Taxol), and thus a second oncology drug (e.g., vincristine, U0126, PJ34, adriamycin, AS703026, 5-Fluorouracil, cisplatin, PLX4720, etc.) different from the first oncology drug (e.g., Taxol) can be administered to the subject. In another example, if the set of proteins or mRNAs are expressed at normal levels in the subject's cancerous tumor relative to the control sample, it is determined that the subject's cancerous tumor is sensitive to the first oncology drug (e.g., Taxol) and thus, the first oncology drug (e.g., Taxol) can be administered to the subject. As there is great biological diversity amongst human tumors, different tumors having different gene signatures and molecular features, the methods described herein are particularly useful for personalized cancer treatment, including predicting a subject's response to a particular drug (e.g., oncology drug), classifying a subject's cancerous tumor, and choosing an appropriate treatment strategy as well as predicting the subject's outcome/survival based on such characterizations.

According to the methods, the drug to which the subject's cancerous tumor is determined to be responsive can be administered to the subject in combination with one or more other oncology drugs and/or treatments (e.g., chemotherapy, radiation therapy, surgery, etc.). In some embodiments, the method can further include determining the subject's prognosis, e.g., outcome, survival, disease-free survival. In such an embodiment, the method further includes correlating overexpression or underexpression of the set of proteins or mRNAs relative to the control sample with a worse prognosis for the subject compared to a second subject having a cancerous tumor in which the first set of proteins are normally expressed relative to the control sample. Generally, what is meant by a “worse prognosis” or “worse outcome/survival” is meant a statistically significant shorter period without relapse, metastasis or death due to tumor.

In these methods, after a subject is treated with a drug, at one or more (e.g., one, two, three, four, etc.) time points, the subject or a sample from the subject (e.g., a biopsy, culture) can be analyzed to determine the subject's response to the drug. In other words, the subject or a sample from the subject (e.g., a biopsy, culture) can be analyzed to determine if the drug is having a therapeutic effect on the subject, e.g., reducing tumor size and/or tumor growth and/or tumor markers. Any suitable methods of analyzing a sample from the subject for the drug's therapeutic effect can be used, including those protein and mRNA assays described herein. Any suitable methods for analyzing the subject to determine if the drug is having a therapeutic effect can be used. Such methods include, for example, physical exams, tumor biomarkers such as CA125, and imaging (x-rays, CT scan, PET scan, MRI etc.).

### Methods and Assays for Predicting Responses of Cancerous Tumors to Drugs and Developing Personalized Therapy for Treatment of Cancerous Tumors

Described herein are methods (e.g., assays) for predicting a response of a cancer patient's cancerous tumor (e.g., ovarian cancer tumor) to a drug (e.g., oncology drug) and developing a personalized therapy for the patient for treatment of the cancerous tumor. Generally, cancerous tumor cells obtained from a patient having a cancerous tumor (e.g., obtained from a biopsy or surgery) are cultured in an appropriate medium (e.g., WIT-OC or WIT-FO medium) and exposed to a particular drug (or to a combination of drugs). The effect of the particular drug (or combination of drugs) on survival and proliferation of the cancerous tumor cells is examined in order to make a prediction of the particular drug's (or the combination of drugs') likely effect on the patient's cancerous tumor. Using the method, a treatment can be selected for the patient based at least in part on a prediction or result suggesting that a particular drug (e.g., oncology drug) will be effective or more effective than one or more alternative drugs for that particular patient. Such methodology can be used to determine a patient-specific response to one or more therapeutic strategies that have been approved for the treatment of the medical condition being treated in the patient (e.g., ovarian cancer), as well as therapies that may be utilized off-label. Use of the prediction methods described herein allows for the identification of optimal personalized treatment strategies for a cancer patient.

In one embodiment, the method includes predicting a response of a cancer patient's (e.g., a female human having an ovarian cancer tumor) cancerous tumor to a drug (e.g., oncology drug) and developing a personalized therapy for the patient for treatment of the cancerous tumor. A typical method includes the steps of: obtaining cancer cells from the patient's cancerous tumor; culturing the cancer cells in WIT-OC cell culture medium (or other WIT culture medium or a derivative of a WIT culture medium); contacting the cultured cancer cells with the drug; determining an IC50 value (or IC90 value—a dose of drug that kills at least 90% of tumor cells) for the drug in the cultured cancer cells; and correlating an increased IC50 (or IC90) value relative to an IC50 (or IC90) value for the drug in control cultured cells with a poor response of the patient's cancerous tumor to the drug and correlating a normal or low IC50 (or IC90) value relative to the IC50 (or IC90) value for the drug in control cultured cells with a positive response of the patient's cancerous tumor to the drug. By a “poor response” is meant no decrease in tumor size or tumor markers. A “positive response” means a decrease in tumor size or tumor markers.

In one embodiment in which the patient's cancerous tumor is not responsive to the drug being tested, the IC50 value is increased relative to the IC50 value for the drug in control cultured cells, and the method further includes administering to the patient a second drug (i.e., a drug different from the drug tested to which the cancerous tumor cells demonstrated a poor response, e.g., Taxol, vincristine, U0126, PJ34, adriamycin, AS703026, 5-Fluorouracil, cisplatin, and PLX4720). In another embodiment, in which the patient's cancerous tumor is responsive to the drug being tested, the IC50 value is normal or decreased relative to the IC50 value for the drug in control cultured cells, and the method further includes administering the tested drug (e.g., Taxol, vincristine, U0126, PJ34, adriamycin, AS703026, 5-Fluorouracil, cisplatin, and PLX4720) to the patient. The method can also be used for making a prognosis for the patient. In such an embodiment, the method can further include correlating an increased IC50 value relative to an IC50 value for the drug in control cultured cells with a worse prognosis for the patient compared to a second patient having a cancerous tumor in which an IC50 value for the drug in cultured cancer cells from the second patient is normal or decreased relative to the IC50 value for the drug in control cultured cells.

Although the experiments described herein involved measuring IC50 or IC90 values, other measurements can be taken to predict a response of a cancer patient's cancerous tumor to a drug. Any assay that measures survival and/or proliferation of cancer cells in response to a drug (e.g., Taxol) can be used. For example, cell number counts, mtt, mtx, alamar blue, apatosis assays, cell cycle profiles, etc. can be used.

As with the other methods described above, the cancer cells may be obtained from a xenograft explant, from ascites fluid, biopsy or primary solid ovarian tissue from the subject. In some embodiments, the method is used to simultaneously predict responses of cancerous tumors from multiple subjects (e.g., 2, 3, 4, 5, 10, 15, 20, 25, 30, 35, 40, 50, 100, etc.) who have cancer to a drug (e.g., oncology drug) or combination of drugs. As already mentioned, a nonexhaustive list of oncology drugs includes Taxol, vincristine, U0126, PJ34, adriamycin, AS703026, 5-Fluorouracil, cisplatin, PLX4720, etc.

As with the other methods described above, after a subject is treated with a drug, at one or more (e.g., one, two, three, four, etc.) time points, the subject or a sample from the subject (e.g., a biopsy, culture) can be analyzed to determine the subject's response to the drug. In other words, the subject or a sample from the subject (e.g., a biopsy, culture) can be analyzed to determine if the drug is having a therapeutic effect on the subject, e.g., reducing tumor size and/or tumor growth and/or tumor markers. Any suitable methods of analyzing a sample from the subject for the drug's therapeutic effect can be used, including those protein and mRNA assays described herein. Any suitable methods for analyzing the subject to determine if the drug is having a therapeutic effect can be used. Such methods include, for example, physical exams, tumor biomarkers such as CA125, and imaging (x-rays, CT scan, PET scan, MRI etc.).

### Methods for Determining the Prognosis of a Subject Having an Ovarian Cancer Tumor

One embodiment of a method for determining a prognosis of a subject (e.g., female human) having an ovarian cancer tumor involves generation of a gene expression signature or profile for the subject's ovarian cancer tumor, and classifying the ovarian cancer tumor as fallopian tube-like or ovary-like. In the experiments described in Example 3 below, a cell-of-origin gene expression signature that distinguishes normal human ovarian (OV) and fallopian tube (FT) epithelial cells within the same subject (e.g., patient) was identified, and it was shown that application of the OV vs. FT cell-of-origin gene signature to gene expression profiles of primary ovarian cancers permits identification of distinct OV and FT-like subgroups among these cancers. The experiments further showed that the normal FT-like tumor classification correlated with a significantly worse disease-free survival, and thus, applying this classification to a gene expression signature or profile of a subject's cancerous tumor can be used for determining a prognosis for the subject (e.g., female human).

In one example of such a method, the method includes the steps of: obtaining a sample from the subject's tumor; subjecting the sample to gene expression profiling resulting in an expression profile including a first set of genes that are upregulated in fallopian tube cells relative to ovarian cells and a second set of genes that are upregulated in ovarian cells relative to fallopian tube cells; determining expression levels of the first and second sets of genes; and correlating an upregulation of the first set of genes and normal expression of the second set of genes with a worse disease-free survival prognosis (e.g., statistically significant shorter period without relapse, metastasis or death due to tumor) relative to a second subject having an ovarian cancer tumor in which the first set of genes are not upregulated and the second set of genes are upregulated. In the method, the first set of genes typically includes all of DOK5, CD47, HS6ST3, DPP6, and OSBPL3, as these genes were found to be overexpressed in cultured fallopian tube cells compared to cultured ovarian cells. If other genes are also found to be overexpressed in cultured fallopian tube cells compared to cultured ovarian cells, the first set of genes can then include one or more (e.g., one, two, three, four, five) of DOK5, CD47, HS6ST3, DPP6, and OSBPL3 in combination with one or more other genes that are overexpressed in cultured fallopian tube cells compared to cultured ovarian cells. The second set of genes typically includes STC2, SFRP1, SLC35F3, SHMT2, and TMEM164, as these genes were found to be overexpressed in cultured ovarian cells compared to cultured fallopian tube cells. If other genes are also found to be overexpressed in cultured ovarian cells compared to cultured fallopian tube cells, the second set of genes can then include one or more (e.g., one, two, three, four, five) of STC2, SFRP1, SLC35F3, SHMT2, and TMEM164, in combination with one or more other genes that are overexpressed in cultured ovarian cells compared to cultured fallopian tube cells. However, any suitable genes can be analyzed, as long as they are differentially expressed between fallopian tube cells and ovarian cells. Quantitative sensitive methods such as PCR and RNA sequencing, for example, can be used to examine other suitable genes that are differentially expressed between fallopian tube and ovary; gene expression profiling can be performed using any suitable methods, including any of those described herein.

In an embodiment in which the first set of genes in the expression profile is upregulated but the second set of genes is not upregulated, the method can further include classifying the subject's ovarian cancer tumor as fallopian tube-like. In another embodiment in which the second set of genes in the expression profile is upregulated but the first set of genes is not upregulated, the method can further include classifying the subject's ovarian cancer tumor as ovary-like. As shown in the experiments described in Example 3 below, fallopian tube-like tumors were of significantly higher stage, higher grade and were predominantly composed of serous adenocarcinomas, while in contrast, ovary-like tumors included non-serous subtypes and lower grade cancers. Thus, the correlation can be made between a subject's ovarian cancer tumor being a fallopian tube-like tumor, and a poor prognosis for the subject. If the subject's ovarian cancer tumor is ovary-like, the subject is expected to have a better prognosis, (a longer period without relapse, metastasis or death due to tumor).

As with the other methods described herein, the method can further include treating the subject with one or more oncology drugs and/or treatments (e.g., chemotherapy, radiation therapy, surgery, etc.). After the subject is treated with a drug, at one or more (e.g., one, two, three, four, etc.) time points, the subject or a sample from the subject (e.g., a biopsy, culture) can be analyzed to determine the subject's response to the drug. In other words, the subject or a sample from the subject (e.g., a biopsy, culture) can be analyzed to determine if the drug is having a therapeutic effect on the subject, e.g., reducing tumor size and/or tumor growth and/or tumor markers. Any suitable methods of analyzing a sample from the subject for the drug's therapeutic effect can be used, including those protein and mRNA assays described herein. Any suitable methods for analyzing the subject to determine if the drug is having a therapeutic effect can be used. Such methods include, for example, physical exams, tumor biomarkers such as CA125, and imaging (x-rays, CT scan, PET scan, MRI etc.).

### Kits

Kits for analyzing sensitivity of a subject's cancerous tumor to an oncology drug (predicting a response of a cancer patient's cancerous tumor to an oncology drug) and developing a personalized therapy for the subject are described herein. A typical kit for determining if a subject's cancerous tumor is sensitive or resistant to a particular oncology drug (e.g., Taxol) includes at least one control such as one more OCI lines as an internal control(s); instructions for use; and WIT medium, or a derivative of WIT medium. Although an OCI line is typically included as a control, any suitable control(s) can be used. Additionally, the kit may contain one or more (e.g., one, two, three, four, five, ten, twenty, etc.) probes. For example, the kit may include one or more probes for use in a multiplexed PCR assay, for example, in which several probes are used simultaneously. Probes that are specific for particular proteins can be used. For example, the one or more probes can be at least two probes specific to at least two (e.g., two, three, four, five, six, etc.) of the following proteins: tubulin, AKT, androgen receptor, Jun oncogene, Crystalline, cyclin D1, epidermal fatty acid binding protein, Ets related gene, FAK, Forkhead Box O3, Erk/Mek, N-cadherin, mitogen-activated protein kinase 14, plasminogen activator inhibitor type 1, paired box 2, protein kinase C-alpha, protein kinase AMP-activated Gamma 2, phosphatase and tensin homolog, SMAD3, Sarcoma viral oncogene homolog, signal transducer and activator of transcription 3, and signal transducer and activator of transcription 5. Optionally, kits may also contain one or more of the following: containers which include positive controls, containers which include negative controls, photographs or images of representative examples of positive results and photographs or images of representative examples of negative results.

### Data and Analysis

Use of the assays, methods and kits described herein may employ conventional biology methods, software and systems. Useful computer software products typically include computer readable medium having computer-executable instructions for performing logic steps of a method. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, for example Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001). See U.S. Pat. No. 6,420,108.

The assays, methods and kits described herein may also make use of various computer program products and software for a variety of purposes, such as reagent design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170. Additionally, the embodiments described herein include methods for providing data (e.g., experimental results, analyses) and other types of information over networks such as the Internet.

### EXAMPLES

The present invention is further illustrated by the following specific examples. The examples are provided for illustration only and should not be construed as limiting the scope of the invention in any way.

### Example 1

### An In Vitro Test for Taxol Sensitivity in Ovarian Tumor Cell Lines that Retain the Phenotype of Primary Tumors

The inability to establish stable cell lines from the vast majority of human tumors has limited the use of in vitro models to study human cancer. Currently available tumor cell lines fail to represent the biological diversity of human tumors. We previously developed a cell culture medium and methods that enabled us to routinely establish cell lines in more than 95% of cases and from diverse subtypes of ovarian tumors. Importantly, the 25 ovarian tumor cell lines described herein retained the genomic landscape and histopathology of the original tumors, and their molecular features.

Described herein is the use of these cell lines to predict a patient's response (including patients' responses) to drugs. We have determined that the drug response of the cell lines we have established correlated with patient outcomes. Thus, tumor cell lines derived using this methodology represent a significantly improved new platform to test and potentially predict patient response to treatment. A robust and efficient model system that predicts patient response to various drugs would greatly improve development of new drugs for personalized treatment of cancer patients. The cell lines we established represent a more malignant, drug-resistant cancer phenotype than has been previously represented in tumor cell line panels. Thus, tumor cell lines derived using this methodology represent a significantly improved new platform to study human tumor biology and treatment.

We previously developed a new culture system for common human cancers both by the ostensible need for improved model systems and by the encouraging results with a new chemically-defined culture medium (WIT) we described previously (Ince et al., Cancer Cell 12, 160-170, 2007). This medium provides all the essential nutrients for maintaining basic cellular metabolism without undefined supplements such as serum, pituitary extract, feeder layers, conditioned medium or drugs (Ince et al., Cancer Cell 12, 160-170, 2007). In WIT medium normal human breast epithelial cells could reach beyond seventy population doublings, a nearly 10²¹-fold expansion of cell numbers. These results encouraged us to hypothesize that perhaps human tumors could also be grown routinely in such a medium.

For the purposes of this report, all ovarian cancer cell lines derived using standard culture medium and methods will collectively be referred to as “standard ovarian carcinoma” cell lines, or SOC cell lines, including the 26 SOC lines available from the American Tissue Type Collection (ATCC) and the European Collection of Cell Cultures (ECACC). The set of ovarian cancer cell lines derived using WIT-OC medium will be referred to as “OCI” cell lines. In two cases the bulk of the tumor mass was located in the fallopian tubes, these cell lines are referred to as “FCI” cell lines.

### Results

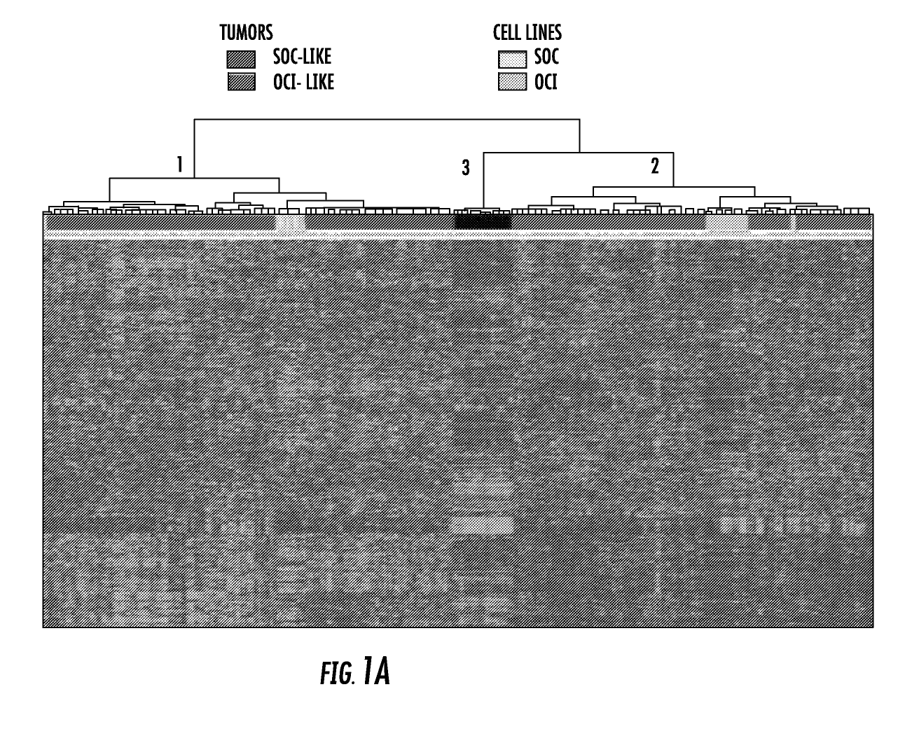

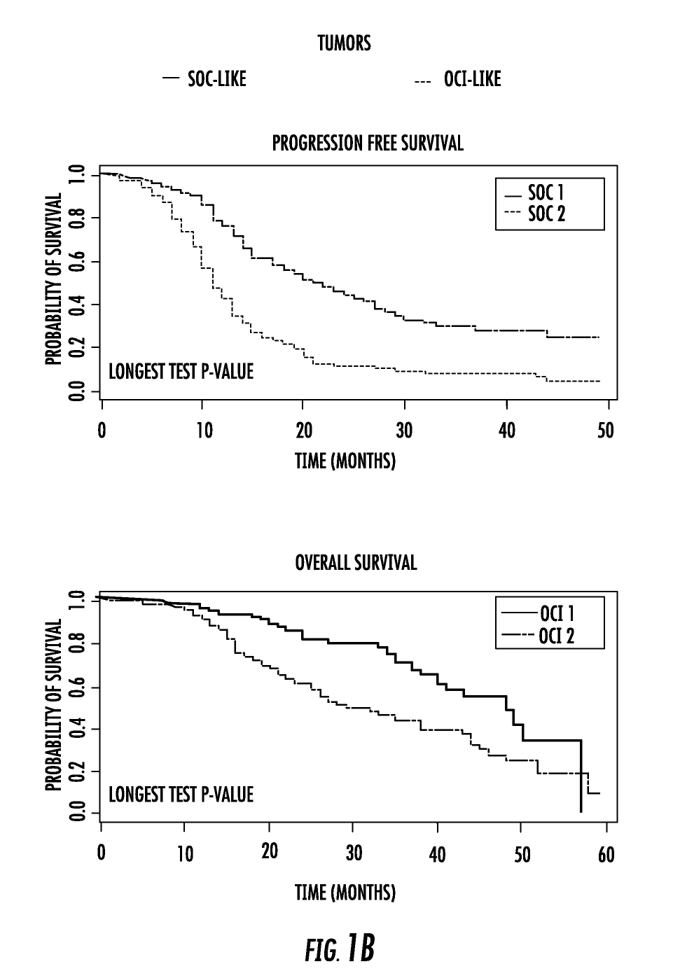

mRNA gene expression profile of the OCI tumor cell lines resembles human tumors with distinct clinical characteristics. Examination of the OCI and SOC cell line panel together with 285 human ovarian tumor specimens revealed three distinct patient clusters. Patient Cluster 1 included only OCI lines, and Cluster 2 included all the SOC lines. None of the cell lines were in Cluster 3 (FIG. 1a). The distribution of the cell lines within human tumor samples was identical to the in vitro cell line clusters, except a single cell line (OCI-C4p), strongly indicating that the in vitro phenotype of these cell lines may reflect relevant in vivo clinical differences. Furthermore, the comparison of the clinical outcomes of these two groups of patients revealed that the patients with OCI-like tumors in Cluster 1 had a significantly shorter progression free and overall survival than tumors in Cluster 2 with an SOC-like profile in multivariate analysis (FIG. 1b).

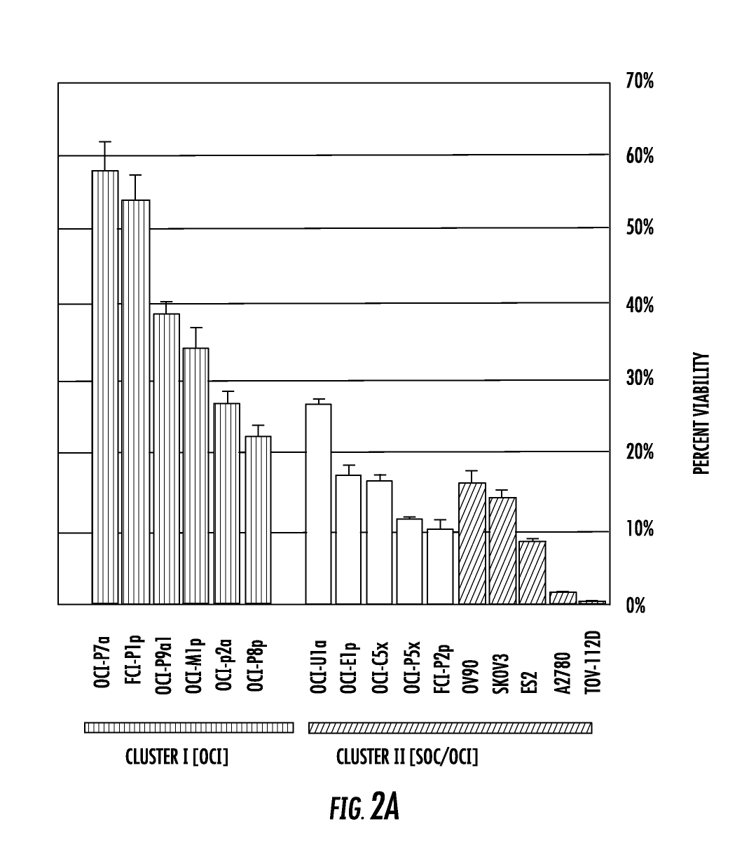

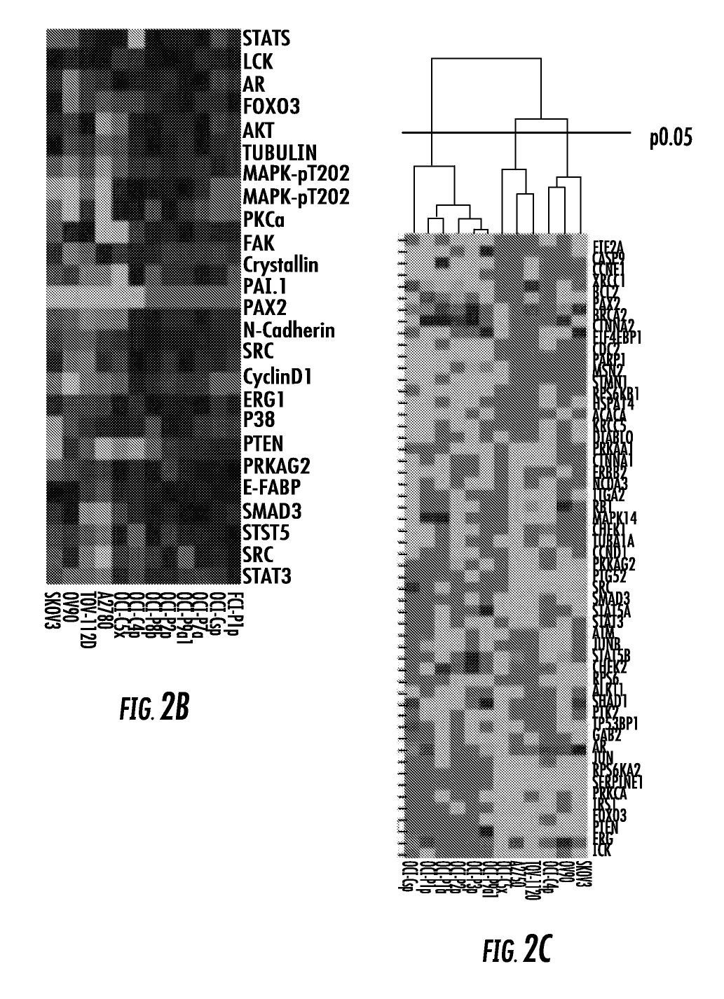

Response of tumor cell lines in mRNA/RPPA Clusters 1 and 2 to Taxol: The striking correlation between poor patient outcomes and OCI lines in mRNA/RPPA Cluster 1 prompted us to test the response of these cell lines to Taxol and Cisplatin, which are two of the most commonly-used drugs for ovarian cancer. We selected a panel of lines that correspond to OCI lines in mRNA/RPPA Cluster 1 and SOC lines in mRNA/RPPA Cluster 2; each panel included examples of different tumor subtypes (PS, CC, CS, E, M), and tissue sources (solid tumors, ascites fluid, and xenograft explants). In these experiments we observed that the IC50 for Taxol in OCI lines in mRNA/RPPA Cluster 1 ranged >10-100 nM, which was >5-10 fold higher than the IC50 values in SOC lines in mRNA/RPPA Cluster 2. The SOC IC50 values for Taxol in these experiments were consistent with previous reports. The subset of OCI lines in Cluster 2 were also more sensitive to Taxol compared to OCI lines in Cluster 1, similar to SOC lines (FIG. 2a). Both OCI and SOC lines were plated in WIT-OC medium for the above experiments. Thus, we infer that the differences in drug response are not a consequence of different growth media. Importantly, we found that the response to another microtubule inhibiting drug, Vincristine, was similarly different between OCI and SOC lines. In contrast, we did not find a significant difference in the response to Cisplatin between OCI and SOC lines.

In order to explore the basis for the relative Taxol resistance of OCI cells we compared the protein profiles Cluster1/OCI cells with Cluster2/SOC lines since they had the largest IC50 differences. There was a strong correlation between protein expression levels and Taxol response of 46 proteins and IC50 values. Among these, we concentrated on 22 proteins that were over-expressed in Cluster 1 (FIG. 2b, Table 1). Reassuringly, Tubulin, which is the target of Taxol, was in this group of proteins. Furthermore, 11 additional proteins in this group had been previously associated with Taxol resistance in disparate studies including AKT, p38, AKT, PTEN, Src, SMAD3, STAT3, STATS. The unsupervised hierarchical clustering of the mRNA microarray data including the list of genes from the resistance-associated protein signature was also able to distinguish identical cell line groups in Clusters 1 and 2 (FIG. 2c). Using functional protein network association software, we found that the majority of these over-expressed proteins either directly or indirectly interact with each other. The amino acid sequences of these proteins are well known in the art.

TABLE 1
The list of proteins that are over-expressed in OCI lines in the mRNA/RPPA
Cluster 1 and associated with Taxol resistance in RPPA analysis.

| RPPA | | Evidence for | |
|---|---|---|---|
| Antibody | | Association with | |
| Probe | Gene Name | Taxol Resistance | Potential Interactome Role |
| a.Tubulin | Tubulin | Murphy et al., | Tubulin over-expressed and mutated in Taxol |
| | | Biochimica et | resistant cells (Sangraijrang Chemotherapy |
| | | Biophysica Acta | (2000)46: 327-334; Orr Oncogene (2003) 22, |
| | | 1784 (2008) | 7280-7295) |
| | | 1184-1191; | |
| | | L'esperance | |
| | | International | |
| | | Journal of | |
| | | Oncology 29: 5- | |
| | | 24, 2006 | |
| AKT | AKT | Lin et al. Br. J. | Rapamycin with paclitaxel displayed synergistic |
| | | Cancer (2003) | effects (Aissat et al., Cancer Chemother |
| | | 88: 973-980; Liu | Pharmacol (2008) 62: 305-313; Liu et al., |
| | | et al., Oncogene | Oncogene (2006c) 25: 3565-3575). |
| | | (2006c) 25: 3565- | Constitutively active Akt contributes to |
| | | 3575; Jiang et al., | Vincristine Resistance (Zhang Cancer |
| | | Drug Resist | Investigation, 28: 156-165, 2010). Akt induces |
| | | Updat. (2008) | survival in paclitaxel treated cells (Bava et al., |
| | | 11(3): 63-76; | The International Journal of Biochemistry & |
| | | Bava 2009 | Cell Biology 43 (2011) 331-341). Akt directly |
| | | | regulates the transcriptional activity of c-Jun |
| | | | (Shin et al., Mol Cancer Res. 2009, 7(5): 745-54) |
| AR.C19. | Androgen | | Androgen receptor is activated by STAT3 |
| | Receptor | | (Ueda J Biol Chem. 2002, 277(9): 7076-85). |
| c.JUN_pS73 | Jun | Paclitaxel- | A physical interaction of Stat3 with c-Jun has |
| | oncogene | resistant Human | been reported both in vitro and in vivo. Stat3 |
| | | Ovarian Cancer | and c-Jun cooperated to yield maximal enhancer |
| | | Cells Undergo c- | function, point mutations of Stat3 within the |
| | | Jun NH2-terminal | interacting domains blocked both physical |
| | | Kinase-mediated | interaction of Stat3 with c-Jun and their |
| | | Apoptosis (Zhou | cooperation (Zhang et al., Mol Cell Biol. 1999, |
| | | Biol Chem. Vol. | 19(10): 7138-46) |
| | | 277, No. 42, | |
| | | 39777-39785, | |
| | | 2002) | |
| Crystalline | Crystalline | | Subunits of crystallin interact with tubulin |
| | | | subunits to regulate the equilibrium between |
| | | | tubulin and microtubules (Houck Clark JI |
| | | | (2010) PLoS ONE 5(7): e11795). |
| Cyclin.D1 | Cyclin D1 | | Cyclin D1 promotes anchorage-independent cell |
| | | | survival by inhibiting FOXO3-mediated anoikis |
| | | | (Gan et al., Cell Death Differ. 2009, 16(10): |
| | | | 1408-1417). |
| E.FABP.C20. | Epidermal | Liu et al., J | E-FABP expression that is blocked by mitogen- |
| | fatty acid | Neurochem. 2008, | activated protein kinase kinase (MEK) inhibitor |
| | binding | 106(5): 2015- | U0126 (Liu et al., J Neurochem. 2008, 106(5): |
| | protein | 2029 | 2015-2029). |
| Erg.1_2_3 | Ets Related | Lu et al., J | Expression of EGR-1 mediated by p38MAPK |
| | Gene | Huazhong Univ | pathway plays a critical role in paclitaxel |
| | | Sci Technolog | resistance of ovarian carcinoma cells (Lu et al., |
| | | Med Sci. 2008, | J Huazhong Univ Sci Technolog Med Sci. 2008, |
| | | 28(4): 451-5 | 28(4): 451-5) |
| FAK_pY397 | FAK | Haider et al., Clin | Docetaxel induces FAK cleavage in taxane- |
| | | Cancer Res | sensitive ovarian cancer cells but not in taxane- |
| | | 2005; 11: 8829- | resistant cells (Haider et al., Clin Cancer Res |
| | | 8836 | 2005; 11: 8829-8836). |
| FOXO3a | Forkhead | | ERK promotes tumorigenesis by inhibiting |
| | Box O3 | | FOXO3a (Yang et al., nature cell biology vol. |
| | | | 10(2), 2008). |
| MAPK_pT202 | Erk/Mek | McDaid Cancer | MEK inhibitor CI-1040 potentiates efficacy of |
| | | Res 65: 2854- | Taxol in xenograft tumor modes (McDaid, |
| | | 2860, 2005; | 2005). RNAi screening identified Erk1 as |
| | | Bauer et al. Breast | enhancing paclitaxel activity (Bauer et al. |
| | | Cancer Research | Breast Cancer Research 2010, 12: R41). |
| | | 2010, 12: R41; Xu | Inactivation of ERK is necessary for the |
| | | 2009 | enhancement of paclitaxel cytotoxicity by |
| | | | U0126 (McDaid Cancer Res 65: 2854-2860, |
| | | | 2005). |
| N.Cadherin | N-Cadherin | Rosano et al., | N-Cadherin is over-expressed in Taxol resistant |
| | | Cancer Res; 17(8); | cells (Rosano et al., Cancer Res; 17(8); 2350- |
| | | 2350-60. 2011 | 60. 2011 AACR). |
| | | AACR | |
| p38_pT180 | Mitogen- | | Constitutive increase of p38-MAPK was found |
| | activated | | in vincristine-resistant cells. Inhibition of p38- |
| | protein | | MAPK by SB202190 reduced increased the |
| | kinase 14 | | sensitivity of cells to chemotherapy (Guo et al., |
| | | | BMC Cancer 2008, 8: 375; Lu et al., J Huazhong |
| | | | Univ Sci Technolog Med Sci. 2008, 28(4): 451- |
| | | | 5). p38 MAP kinase phosphorylates c-Jun (Lo et |
| | | | al., Mol Nutr Food Res. 2007, 51(12): 1452-60). |
| PAI.1 | Plasminogen | | MEK/ERK1/2 and SMAD3 was essential for |
| | activator | | PAI-1 induction initiated by microtubule |
| | inhibitor | | disruption (Samarakoon et al., Cell Signal. 2009 |
| | type 1 | | June; 21(6): 986-995). |
| PAX2 | Paired Box 2 | Buttiglieri 2003 | PAX2 expression correlated with enhanced |
| | | | resistance against apoptotic signals and with the |
| | | | proinvasive phenotype (Buttiglieri 2003). |
| PKCa | Protein | | Purified protein kinase C phosphorylates |
| | Kinase C - | | microtubule-associated protein 2. (Akiyama et |
| | alpha | | al., J Biol Chem (1986) Vol. 261, No. 33, |
| | | | 15648-15651). |
| PRKAG2 | Protein | | |
| | Kinase | | |
| | AMP- | | |
| | Activated | | |
| | Gamma 2 | | |
| PTEN.138G50. | Phosphatase | | Silencing Akt in PTEN-mutated prostate cancer |
| | and Tensin | | cells enhances the antitumor effects of Taxol |
| | homolog | | (Priulla et al., The Prostate 67: 782-789 (2007)). |
| SMAD3 | SMAD3 | Increased | SMAD3 binds to microtubules (Dong et al., |
| | | expression in | Molecular Cell, Vol. 5, 27-34, 2000). SMAD3 |
| | | Paclitaxel resistant | and SMAD4 cooperate with c-Jun/c-Fos to |
| | | cells (Kashkin et | mediated transcription (Zhang et al., Nature. |
| | | al., Doklady | 1998, 394(6696): 909-13). |
| | | Biochemistry and | |
| | | Biophysics, 2011, | |
| | | Vol. 437, pp. 105- | |
| | | 108) | |
| SRC | Sarcoma | Knockdown of Src | SRC activates STAT3 (Cao 1996). STAT3 |
| | viral | enhanced | siRNA inhibited Bcl-2 expression (Choi et al., |
| | oncogene | paclitaxel- | Exp Mol Med. 2009, 41(2): 94-101). Bcl-2 |
| | homolog | mediated growth | down-regulation is associated with Paclitaxel |
| | | inhibition in | reesistance (Ferlini et al., Molecular |
| | | ovarian cancer | Pharmacology Vol. 64, No. 1, 51-58, 2003). |
| | | cells (Le et al., | Constitutive activation of Stat3 by the Src |
| | | Cancer Biology & | causes growth of breast carcinoma cells (Garcia |
| | | Therapy 12: 4, | Oncogene (2001) 20, 2499-2513). Dasatinib |
| | | 260-269, 2011; | has synergistic activity with paclitaxel in |
| | | Chen 2005) | ovarian cancer cells (Teoh, et al. Gynecologic |
| | | | Oncology 121 (2011) 187-192). |
| STAT3 | Signal | STAT3 activation | STAT3 is activated by ERK1 and and induces |
| | Transducer | through Src leads | AKT. STAT3 binds the C-terminal tubulin (Ng |
| | and | to Taxol resistance | et al Biochem J. 2009, 425(1): 95-105). |
| | Activator of | (Hawthorne Mol | Knockdown of Stat3 reduces AKT1 expression |
| | Transcription 3 | Cancer Res 2009, | (Park et al., J Biol Chem. Vol. 280, No. 47, |
| | | 7(4)) | 38932-38941, 2005) STAT3 is induced by |
| | | | Src (Zhang et al., JBC Vol. 275, No. 32, |
| | | | 24935-24944, 2000). |
| STAT5 | Signal | | STAT5 was shown to activate cyclin D1 gene |
| | Transducer | | expression (Magne et al., Mol Cell Biol. 2003, |
| | and | | 23(24): 8934-45). |
| | Activator of | | |
| | Transcription 5 | | |

TABLE 2
Proteins with Taxol resistance association over-expressed in Cluster 1
Proteins with Taxol resistance association over-expressed in Cluster 1

| | Function and Association with | |
|---|---|---|
| | Taxol Resistance | Reference |
| a. Tubulin | Tubulin: Target for binding of | Murphy et al., |
| | Taxol. | Biochimica et |
| | | Biophysica Acta |
| | | 1784 (2008) 1184- |
| | | 1191; |
| | | L'esperance |
| | | International Journal |
| | | of Oncology 29: 5- |
| | | 24, 2006 |
| AKT | Rapamycin with paclitaxel | Akt induces |
| | displayed synergistic | survival in |
| | effects | paclitaxel treated |
| | (Aissat 2008; Liu 2006; Zhang | cells (Bava |
| | 2010; Priulla 2007) | 2011). |
| | Akt directly regulates the | |
| | transcriptional activity of | |
| | c-Jun (Shin 2009) | |
| c.JUN_pS73 | Stat3 and c-Jun cooperate to | |
| | yield maximal enhancer function. | |
| Cyclin.D1 | Cyclin D1 promotes anchorage- | (GAN 2009). |
| | independent cell survival by | |
| | inhibiting FOXO3-mediated | |
| | anoikis | |
| E.FABP.C20. | E-FABP expression that is | Liu 2008 |
| | blocked by mitogen-activated | |
| | protein kinase kinase (MEK) | |
| | inhibitor U0126. | |
| Erg.1_2_3 | | Lu 2008 |
| FAK_pY397 | | Haider 2005 |
| FOXO3a | ERK promotes tumorigenesis | (Yang 2008) |
| | by inhibiting FOXO3a | |
| MAPK_pT202 | | McDaid 2005, Xu |
| | | 2009 |
| N.Cadherin | | Rosano 2011 |
| P38_pT180 | Constitutive increase of | (Guo 2008, Lu |
| | p38-MAPK was found in | 2008, Lo 2007) |
| | vincristine-resistant cells. | |
| | Inhibition of p38-MAPK by | |
| | SB202190 increased the | |
| | sensitivity of cells to | |
| | chemotherapy | |
| PAX2 | | Buttiglieri 2003 |
| PTEN.138G50. | | Priulla 2007 |
| SMAD3 | SMAD3 binds to microtubules | Kashkin 2011 |
| | (Dong 2000) SMAD3 and SMAD4 | |
| | cooperate with c-Jun/c-Fos to | |
| | mediated transcription | |
| | (Zhang 1998) | |
| SRC | SRC activates STAT3 (Cao 1996) | Teoh 2011, Le |
| | STAT3 siRNA inhibited Bcl-2 | 2011, Fournier |
| | expression (Choi 2009). | 2011, Chen |
| | Constitutive activation of | 2005, |
| | Stat3 by the Src causes growth | Hawthorne 2009 |
| | of breast carcinoma cells | |
| | (Garcia 2001). | |
| STAT3 | STAT3 is activated by ERK1 | Hawthorne 2009 |
| | and induces AKT. STAT3 binds | |
| | the C-terminal tubulin (Ng 2009). | |
| | Knockdown of Stat3 reduces | |
| | AKT1 expression (Park 2005) | |

As described in FIGS. 1 and 2, we developed a test to tell which patients will respond to Taxol, which is the first line drug for ovarian cancer (it is also used for many other cancers including breast). In one embodiment this test may be in the form of analyzing the expression of the proteins that are listed in FIG. 1 in patient tumors. In another embodiment, it may take the form of making cell lines from patients and carrying out an in vitro test to determine the IC50 on the cell lines as we show in these figures.

### Example 2

### Characterization of Novel Ovarian Tumor Cell Lines that Retain the Phenotype of Primary Tumors

Currently available human tumor cell line panels consist of a small number of lines that generally fail to retain the phenotype of the original patient tumor. We have developed a cell culture medium that enables us to routinely establish cell lines from diverse subtypes of human ovarian cancers with >95% percent efficiency. Importantly, the 25 ovarian tumor cell lines described here retained the genomic landscape, histopathology, and molecular features of the original tumors. Furthermore, the molecular profile and drug response of these cell lines correlated with distinct groups of primary tumors with different outcomes. Thus, tumor cell lines derived using this new methodology represent a significantly improved new platform to study human tumor biology and treatment.

Human carcinomas that grow uncontrollably in the body are paradoxically difficult to grow in cell culture. A robust and efficient cell line model system that predicts a patient's response to various drugs would greatly improve development of new drugs for personalized treatment of cancer patients. The cell lines we established capture the in vivo heterogeneity of human ovarian tumors and correspond to a more malignant, drug-resistant cancer phenotype than standard ovarian cancer cell lines.

We set out to develop a new culture system for common human cancers, driven both by the clear need for improved in vitro models and by the encouraging results with the WIT medium that we described previously (Ince et al., Cancer Cell 12, 160-170, 2007). These results encouraged us to hypothesize that perhaps human tumors could also be grown consistently in such a medium. This report characterizes the phenotypic properties of 25 new continuous OCI derived using cell culture media (WIT-OC) optimized for human ovarian cancer subtypes.

### Results

Tumor cells fail to thrive in standard cell culture media. Consistent with prior reports, we were able to establish tumor cell lines in standard culture media only with less than one percent success rate. In the single successful case, the ovarian tumor line OCI-U1a was derived in RPMI medium; in which a brief period of rapid growth (days 0-20), was followed by growth arrest (days 20-40), widespread cell death (days 40-50), and the eventual emergence of rapidly growing rare clones that gave rise to a continuous cell line (days 60-90). Importantly, the copy number variants (CNV) measured in the genome of the cell line grown in RPMI varied significantly from those found in the starting tumor cell population, consistent with clonal outgrowth of select subpopulations or the acquisition of additional genetic aberrations during tissue culture. Consistent with the experience of others in this field, over the course of this nearly ten year-long study, this was the only patient tumor specimen that yielded a continuous ovarian tumor cell line using standard media.

Optimization of culture conditions for ovarian tumor cells in WIT medium. We also discovered that the original WIT medium developed for breast cells (Ince et al., Cancer Cell 12, 160-170, 2007) did not support the growth of either normal ovarian cells or tumors of the ovary. Normal human breast cells, like most normal epithelium, are never in direct contact with blood or serum under physiologic conditions. Accordingly, the medium we developed for normal breast cells was completely devoid of serum in order to more closely approximate the physiologic environment. In contrast, normal ovarian and fallopian tube epithelial cells are known to be directly in contact with normal peritoneal fluid, which contains a physiologic serum protein concentrations that can be as high as fifty percent of the levels present in the circulating blood. Indeed, in many cases the ovarian tumors grow in malignant ascites fluid that has concentrations of proteins and growth factors that are actually higher than those present in serum. Thus, we added serum into WIT medium in order to mimic the physiologic growth conditions of malignant ovarian cells. However, supplementation of WIT medium with serum was not sufficient for growth of ovarian tumor cells, without additional factors. After testing many modifications over the years, we found that a combination of factors including particular concentrations of serum, insulin, hydrocortisone, EGF, cholera toxin, estrogen were also necessary but not sufficient to culture different ovarian tumor types with high efficiency. In addition to medium optimization, it was necessary to optimize O₂ levels and the cell attachment surfaces, because while endometrioid and mucinous histotypes of ovarian tumors were best cultured in low O₂ conditions (5-10%), the serous, clear cell and other subtypes proliferated best in ambient O₂ (18-21%). Lastly, we found that a modified plastic surface (Primaria, BD) performed best for the culture of primary ovarian tumors.

It is worth noting that during the course of this work we discovered that optimizing individual culture medium variables one at a time resulted in small improvements in culture success. Nevertheless, several components that had little effect on culture success by themselves had a large combined effect. However, these synergistic effects were difficult to predict and empirically testing all possible combinations of multiple components was prohibitive due to very large number of permutations. In addition, optimization of conditions on one tumor sample did not ensure universal success, since a particular variable was sometimes dispensable for some tumor samples, and absolutely essential for the successful culture of others. Lastly, effects of changing culture conditions or medium formulation sometimes became apparent after successive passages. Thus, a very time consuming aspect of this process was the need to test combinations of variables in multiple primary ovarian cancer samples over many passages to ensure broad applicability across all ovarian cancer subtypes. These four factors: (1) the non-obvious nature of synergistic combinatorial effects of medium components, (2) the need to test long-term effects of each variable over many months, (3) the necessity to test each variable in multiple tumor samples, and (4) the very large number of permutations to test, precluded an incremental systematic approach to the development of WIT-OC media. Nevertheless, once the medium and cell culture conditions were optimized, only one sample out of twenty-six failed to generate a cell line.

OCI cell lines encompass the major histological subtypes of ovarian cancer. Adenocarcinoma of the ovary is a heterogeneous disease that is comprised of many histopathological subtypes with distinct features. In many cases the original subtype of tumor that gave rise to most of the “standard ovarian carcinoma” (SOC) cell lines is unknown. In this study we used the small subset of SOC cell lines in which the histologic subtype is known. In order to distinguish the cell lines derived using WIT-OC medium from SOC lines, they will be referred to as “OCI” cell lines. In two cases the bulk of the tumor was located in the fallopian tubes—these cell lines are referred to as “FCI” cell lines. The capital letter after the ovarian carcinoma designation “OCI” refers to the histological subtype of the original tumor. The OCI panel includes papillary serous (P), clear cell (C), endometrioid (E), mucinous (M) cancers, and rare types such as carcinosarcoma (CS) and dysgerminoma (D). Together, the P, C, E and M subtypes account for more than ninety percent of ovarian adenocarcinomas; accordingly this panel of cell lines is broadly representative of ovarian cancer. The lower case letter at the end of each cell line name refers to tissue source; 14 of the cell lines were established from primary solid tumors (p), seven from ascites fluid (a), and four from primary mouse xenografts derived from direct implantation of human tumors into immunocompromised mice (x). All 25 OCI lines were able to form colonies in soft agar, consistent with retention of a transformed phenotype in culture.

In WIT-OC medium the tumor cells were able to proliferate immediately, suggesting that most of the tumor cells proliferated without significant in vitro clonal selection or a need to acquire additional genomic or epigenetic aberrations. Furthermore, it was possible to culture these cells continuously for 30-100 population doublings with no decrease in growth rate; we have not yet identified an upper limit of population doublings.

Standard media fail to support OCI cell lines. We observed that none of the OCI lines we tested could be cultured in existing standard media. In contrast, all of the SOC lines we tested could be cultured in WIT-OC medium. Until now, none of the standard media support the culture of all of the existing SOC lines, making it difficult to compare a large panel of SOC lines with one another because they require being cultured in a variety of different media. Our results indicate that WIT-OC medium has the potential to serve as a universal culture medium for SOC lines facilitating comparisons across cell lines.

OCI cell lines mirror the genomic landscape of the original tumor. Major genetic alterations may accumulate during cell culture in standard media. In order to compare tumor vs. cell line genomes, we examined their loss-of-heterozygosity (LOH) profiles and found that each OCI cell line exhibited remarkable similarity to its corresponding uncultured tumor sample. In several cases there were especially striking similarities between the cell line and tumor (OCI-M1p/TM1, OCI-P2a/TP2, OCI-C2p/TC2, OCI-EP1/TEP1). Two of the OCI lines (OCI-U1p and P5x) and their matched tumors had large-scale alterations that involved whole chromosome arms. In contrast, the remaining ten OCI lines and their matched tumors contained genomic regions of LOH that spanned narrow regions of the chromosomes. There was a more than 90% identity between the LOH pattern of the uncultured tumor and the matching cell lines, except in four cases, there were significant differences in the LOH profile between cultured cells and the primary tumor. The overall CVN trends were similar between OCI lines and ovarian tumors; in both data sets CNV trend was copy number gain in chromosomes 2, 19, 20 and copy number loss in chromosomes 4, 9, 13, 15, and 18. The remaining chromosomes had a more complex pattern. Interestingly, while copy number losses were predominant in short arm of chromosomes 3 and 8, gains were predominant in the long arm, and this pattern was replicated in OCI lines. These results are consistent with the LOH comparison between OCI lines and their matched tumors and indicate that the genomic landscape of primary tumors are preserved in OCI lines. Because the fragment of tumor from which the cell line is established is necessarily different than the fragment of tumor from which the DNA is isolated, it is possible that intra-tumoral genetic heterogeneity may be responsible for some of these differences. It is also possible that in a few cases some of the changes may be due to accumulation of genetic alterations during culture, even though there was no noticeable difference in the growth rate among these lines and the other OCI lines.

We were not able to compare the DNA of the SOC lines to the matched original tumor DNA because these cells were established decades ago and the original tumor sample is not available. Next we compared copy number variation (CNV) patterns of OCI cells with the ovarian tumors analyzed in the TCGA dataset. Consistent with the LOH analysis, the overall CNV trends of the OCI lines was similar to primary tumor samples.

A persistent problem in the cell culture field has been cross-contamination and misidentification of lines, existing in up to 15-20% of published reports. The close genomic match between OCI lines and the original tumor tissues ensure that the OCI lines were each derived from a unique patient. Furthermore, we sequenced the mitochondrial DNA of the OCI and SOC lines to provide a permanent unique identifier for authentication of these cell lines. Overall, these results indicate that a majority of the OCI cell lines faithfully preserve the genetic alterations present in the tissue.

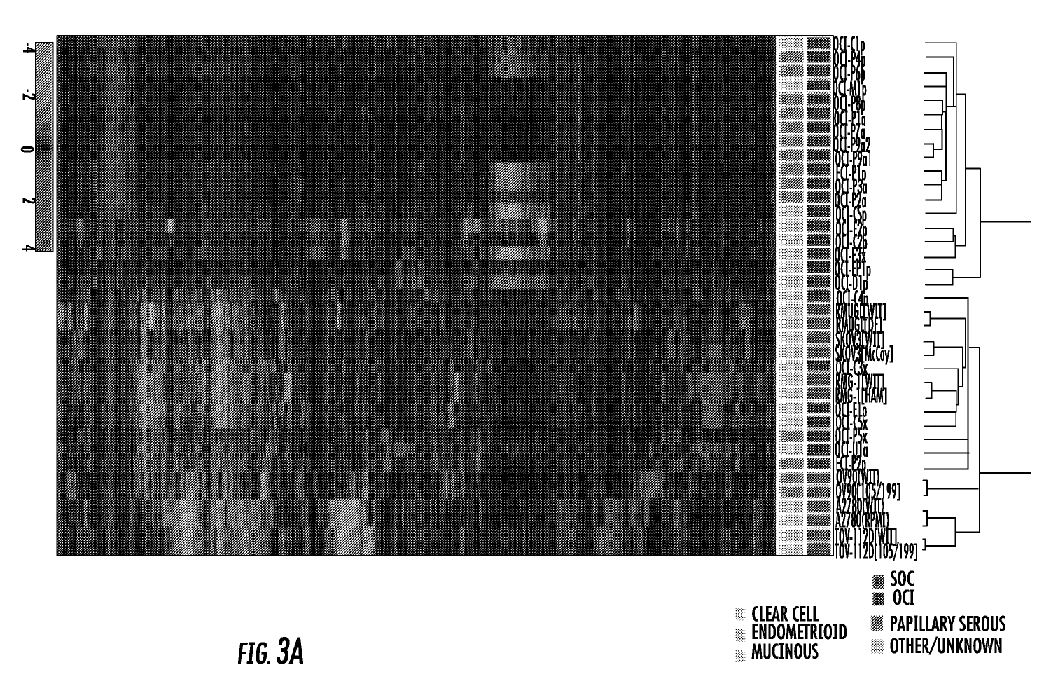

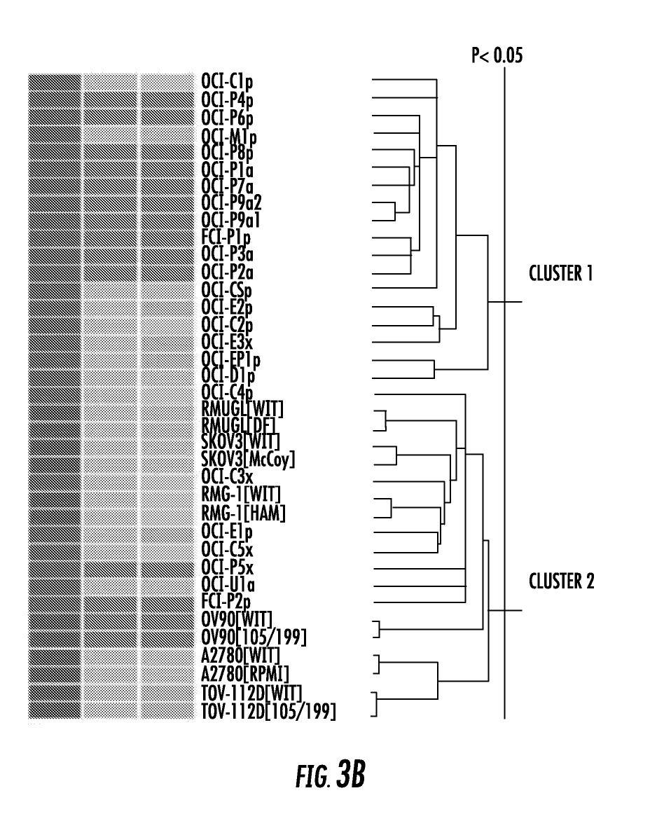

OCI and SOC lines possess different gene expression signatures. Unsupervised hierarchical clustering of the mRNA expression data of 25 OCI and six SOC lines revealed two major clusters; 558 and 265 genes were found up-regulated in clusters 1 and 2 respectively (FIG. 3, Table 3).

TABLE 3
List of 20 most up and down regulated mRNAs in Cluster
1. The complete dataset is available through the NIH's
Gene Expression Omnibus, GEO accession number GSE40785.

| | | fold |
|---|---|---|
| Cluster#1, Up regulated genes | | |
| HSD11B1 | hydroxysteroid (11-beta) dehydrogenase 1 | 32.20 |
| DIRAS3 | DIRAS family, GTP-binding RAS-like 3 | 27.35 |
| HSD11B1 | hydroxysteroid (11-beta) dehydrogenase 1 | 23.89 |
| HSD11B1 | hydroxysteroid (11-beta) dehydrogenase 1 | 22.85 |
| COL1A2 | collagen, type I, alpha 2 | 22.61 |
| COL1A2 | collagen, type I, alpha 2 | 19.01 |
| COL1A1 | collagen, type I, alpha 1 | 12.53 |
| THBS2 | thrombospondin 2 | 11.51 |
| ANXA8 | annexin A8 | 11.46 |
| BDKRB1 | bradykinin receptor B1 | 11.34 |
| RARRES1 | retinoic acid receptor responder 1 | 9.23 |
| COL5A1 | collagen, type V, alpha 1 | 9.11 |
| C13orf33 | chromosome 13 open reading frame 33 | 8.58 |
| ANXA8 | annexin A8 (ANXA8) | 8.40 |
| SPARC | secreted protein, acidic, cysteine-rich | 8.27 |
| | (osteonectin) | |
| SERPINE1 | serpin peptidase inhibitor 1 | 8.18 |
| CPA4 | carboxypeptidase A4 | 7.85 |
| RARRES1 | retinoic acid receptor responder 1 | 7.80 |
| WISP1 | WNT1 inducible signaling pathway protein 1 | 7.74 |
| LOC652846 | imilar to Annexin A8 (Vascular | 7.53 |
| | anticoagulant-beta) | |
| Cluster #1, Down regulated genes | | |
| CD24 | CD24 molecule (CD24) | 0.17 |
| QPRT | quinolinate phosphoribosyltransferase | 0.29 |
| IGF2BP3 | insulin-like growth factor 2 mRNA binding | 0.32 |
| | protein 3 | |
| PITX1 | paired-like homeodomain transcription factor 1 | 0.35 |
| SPINT2 | serine peptidase inhibitor, Kunitz type, 2 | 0.38 |
| IMPA2 | inositol(myo)-1(or 4)-monophosphatase 2 | 0.40 |
| CLDN1 | claudin 1 | 0.41 |
| CTSL2 | cathepsin L2 | 0.41 |
| HIST1H4C | histone cluster 1, H4c | 0.43 |
| ABLIM1 | actin binding LIM protein 1 | 0.43 |
| AURKB | aurora kinase B | 0.43 |
| CMTM8 | CKLF-like MARVEL TM 8 | 0.43 |
| SDC1 | syndecan 1 (SDC1), transcript variant 1 | 0.44 |
| UBE2C | ubiquitin-conjugating enzyme E2C | 0.44 |
| AIF1L | allograft inflammatory factor 1-like | 0.44 |
| HES4 | hairy and enhancer of split 4 (Drosophila) | 0.44 |
| UBE2C | ubiquitin-conjugating enzyme E2C | 0.44 |
| CDCA5 | cell division cycle associated 5 | 0.44 |
| DBNDD1 | dysbindin, dystrobrevin binding protein 1 | 0.44 |
| CDCA7 | cell division cycle associated 7 | 0.45 |

TABLE 4
List of 20 up and down regulated mRNAs in Cluster 2.
The complete dataset is available at the NIH's Gene
Expression Omnibus, GEO accession number GSE40785.

| | | fold |
|---|---|---|
| Cluster #2, Up regulated genes | | |
| TACSTD1 | tumor-associated calcium signal transducer 1 | 12.42 |
| EPCAM | epithelial cell adhesion molecule | 9.81 |
| SPINT2 | serine peptidase inhibitor, Kunitz type, 2 | 9.24 |
| S100A4 | S100 calcium binding protein A4 | 8.67 |
| TACSTD2 | tumor-associated calcium signal transducer 2 | 7.71 |
| CDH1 | cadherin 1, type 1, E-cadherin | 6.49 |
| MAL | mal, T-cell differentiation protein | 6.16 |
| ZIC2 | Zinc family member 2 (Drosophila) | 6.03 |
| C10orf58 | chromosome 10 open reading frame 58 | 5.98 |
| MAL2 | mal, T-cell differentiation protein 2 | 5.94 |
| S100A4 | S100 calcium binding protein A4 | 5.93 |
| UGT2B7 | UDP glucuronosyltransferase 2 family, | 5.72 |
| | polypeptide B7 | |
| APOE | apolipoprotein E | 5.71 |
| SPP1 | secreted phosphoprotein 1 | 5.70 |
| GLDC | glycine dehydrogenase | 5.48 |
| SPP1 | secreted phosphoprotein 1 | 5.44 |
| UCP2 | uncoupling protein 2 (mitochondrial, proton | 5.39 |
| | carrier) | |
| MDK | midkine (neurite growth-promoting factor 2) | 5.39 |
| ALDH1A1 | aldehyde dehydrogenase 1 family, member A1 | 5.13 |
| FAM84B | family with sequence similarity 84, member B | 5.08 |
| Cluster #2, Down regulated genes | | |
| TMEM98 | transmembrane protein 98 | 0.07 |
| EFEMP1 | EGF-containing fibulin-like extracellular | 0.07 |
| | matrix protein 1 | |
| DCN | decorin (DCN), transcript variant C | 0.07 |
| CDH11 | cadherin 11, type 2, OB-cadherin (osteoblast) | 0.09 |
| MT1E | metallothionein 1E (MT1E) | 0.10 |
| COL3A1 | collagen, type III, alpha 1 (COL3A1) | 0.10 |
| ALDH1A3 | aldehyde dehydrogenase 1 family, member A3 | 0.10 |
| IGFBP4 | insulin-like growth factor binding protein 4 | 0.11 |
| COL5A2 | collagen, type V, alpha 2 | 0.11 |
| PDPN | podoplanin (PDPN) | 0.12 |
| PLOD2 | procollagen-lysine, 2-oxoglutarate | 0.12 |
| | 5-dioxygenase 2 | |
| SPOCK1 | sparc/osteonectin, (testican) 1 | 0.12 |
| FLNC | filamin C, gamma (actin binding protein 280) | 0.12 |
| CRISPLD2 | cysteine-rich secretory protein LCCL domain | 0.14 |
| | containing 2 | |
| DKK3 | dickkopf homolog 3 (Xenopus laevis) ( | 0.14 |
| RAC2 | rho family, small GTP binding protein Rac2 | 0.14 |
| SGK1 | serum/glucocorticoid regulated kinase 1 | 0.15 |
| DPYSL3 | dihydropyrimidinase-like 3 | 0.15 |
| SGK1 | serum/glucocorticoid regulated kinase 1 | 0.15 |
| PLIN2 | perilipin 2 | 0.15 |

Cluster 1 contained only OCI lines (FIG. 3a-b). Most of the OCI papillary serous lines were in this cluster (10/12) (FIG. 3a-b, Cluster 1). In contrast, Cluster 2 was predominantly composed of non-papillary serous tumors (10/13), and contained the entire SOC panel of cell line samples (12/12) (FIG. 3a-b).

Consistent with the above results, others have shown that while mRNA profiles of human serous cancers constitute a distinct group, the profiles of a small subset of endometrioid and clear cell tumors overlap those of papillary serous tumors. Reminiscent of this pattern, some endometrioid and clear cell OCI lines were associated with papillary serous-dominant Cluster 1, suggesting that some tumors classified as endometrioid and clear cell histologically may have a papillary serous-like gene expression signature (FIG. 3b, Cluster 1). The mRNA expression profile of OCI lines in Cluster 2 resembled the SOC lines (FIG. 3a-b). Notably, three out of four xenograft-derived OCI-lines were in Cluster 2 (FIG. 3a-b, C3x, C5x, and P5x), suggesting that the cell lines derived from xenograft explants (Cluster 2) have distinct expression profiles compared to cell lines established from primary tumors (Cluster 1).

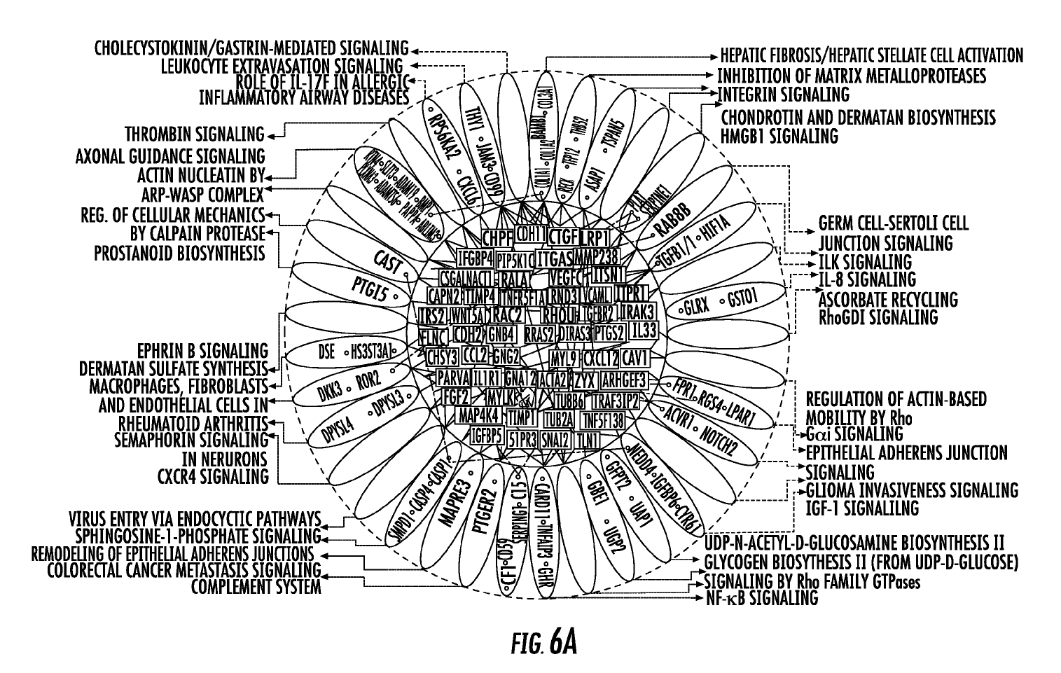

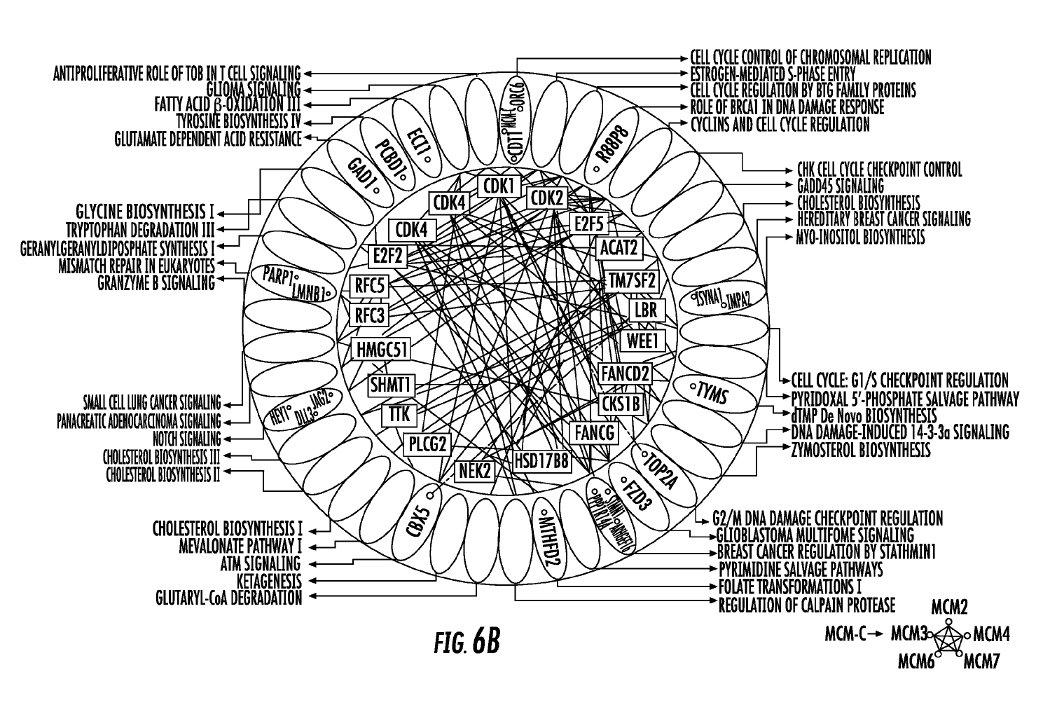

Importantly, SOC lines that were cultured in both standard media and WIT-OC clustered next to one another, indicating that the differences observed between OCI and SOC lines were not due to differences in constituents contained in the culture medium. (FIG. 3b). The mRNA probes that were up-regulated and down regulated in cluster 1 vs. cluster 2 involved 37 and 41 pathways respectively (p<0.05) (FIG. 6a); including NF-kB, CXCR4, IGF-1, Rho-GDI, ILK, and IL-8 signaling (cluster 1); Notch, BRCA, GADD45, Granzyme and Stathmin signaling (cluster 2) among others (FIG. 6b). In summary, Cluster 1 generally correlated with papillary serous histology, OCI cell lines and primary tumor-derived lines; Cluster 2 correlated with non-papillary serous histology, SOC cell lines and xenograft explants.

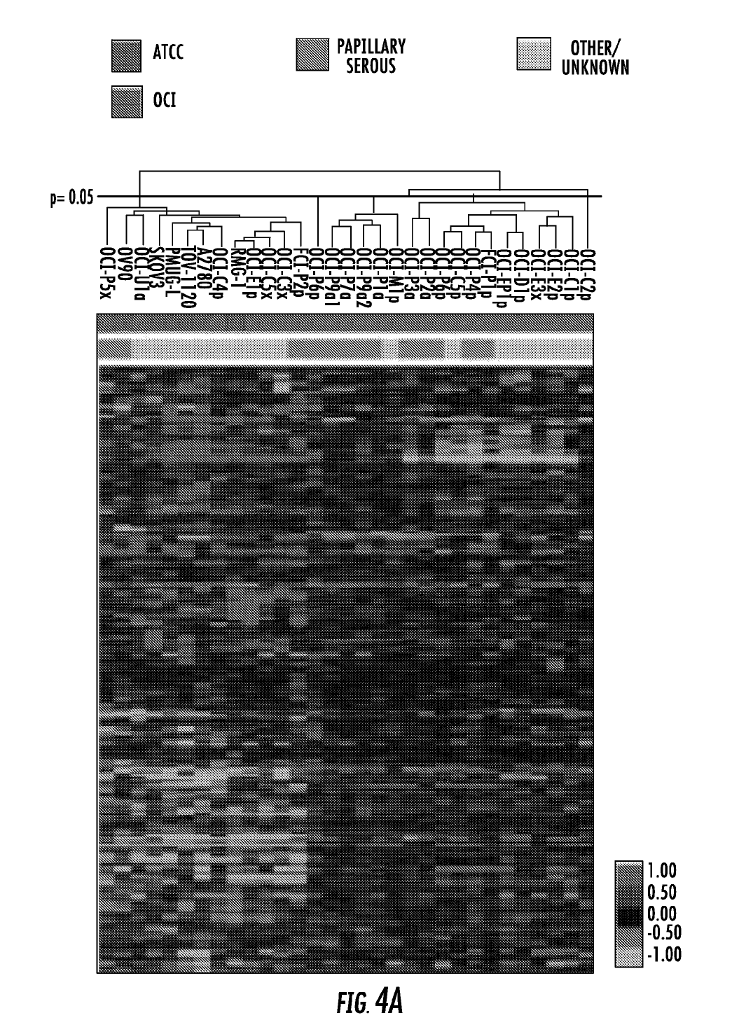

Protein and mRNA expression profiles identify the same OCI cell line clusters. We next examined the expression levels of 226 proteins representing major signaling pathways using Reverse Phase Protein Analysis (RPPA). The unsupervised hierarchical clustering of the protein expression data revealed two major clusters; once again Cluster 1 contained only OCI lines as well as most of the papillary serous lines (10/12) (FIG. 4). In contrast, Cluster 2 was predominantly non-papillary serous lines (10/13), and contained all of the SOC lines (6/6) (FIG. 4). See Tables 5 and 6 for lists of differentially expressed genes between Clusters 1 and 2. To assess reproducibility of these phenotypes, the protein extracts were prepared in triplicate from three different passages across two experiments; in each case we observed that the replicates from each cell line clustered together. Comparison of the cell lines' mRNA and RPPA profiles also revealed a remarkable degree of consistency in molecular phenotypes. The mRNA and RPPA clusters were identical with one exception (OCI-C4p). These results indicate that the molecular differences between OCI vs. SOC lines are stable and reproducible across passages and analytical platforms.

TABLE 5
PATHWAYS UPREGULATED IN CLUSTER 1

| Ingenuity Canonical | |
|---|---|
| Pathways | Molecules |
| Hepatic Fibrosis/ | IGFBP4, VCAM1, CTGF, TNFRSF1A, FGF2, |
| Hepatic Stellate | ACTA2, BAMBI, VEGFC, IGFBP5, IL1R1, |
| Cell Activation | MYL9, TGFBR2, COL1A2, COL1A1, CCL2, |
| | TIMP1, COL3A1 |
| Inhibition of | TIMP4, MMP23B, TIMP1, RECK, THBS2, |
| Matrix | TFPI2, LRP1 |
| Metalloproteases | |
| Integrin Signaling | RAC2, PARVA, RALA, TSPAN5, ASAP1, |
| | DIRAS3, ACTA2, ITGA5, TLN1, MYLK, |
| | MYL9, RRAS2, RND3, RHOU, CAV1, ZYX, |
| | CAPN2 |
| Chondroitin and | CHSY3, CHPF, CSGALNACT1 |
| Dermatan | |
| Biosynthesis | |
| HMGB1 Signaling | VCAM1, RRAS2, CCL2, RND3, TNFRSF1A, |
| | DIRAS3, RHOU, IL1R1, SERPINE1, PLAT |
| Germ Cell-Sertoli | TGFBR2, RAC2, CDH2, RRAS2, RND3, |
| Cell Junction | TUBB6, TNFRSF1A, DIRAS3, TUBB2A, |
| Signaling | ACTA2, RHOU, ZYX, RAB8B |
| ILK Signaling | PARVA, TNFRSF1A, DIRAS3, ACTA2, |
| | VEGFC, HIF1A, MYL9, TGFB1I1, RND3, |
| | FLNC, SNAI2, RHOU, IRS2, PTGS2 |
| IL-8 Signaling | RAC2, VCAM1, DIRAS3, VEGFC, MAP4K4, |
| | IRAK3, GNAI2, MYL9, GNB4, RRAS2, |
| | RND3, RHOU, GNG2, PTGS2 |
| Ascorbate Recycling | GLRX, GSTO1 |
| (Cytosolic) | |
| RhoGDI Signaling | DIRAS3, ACTA2, ITGA5, CDH11, GNAI2, |
| | MYL9, GNB4, CDH2, RND3, PIP5K1C, |
| | RHOU, GNG2, ARHGEF3 |
| Regulation of | MYLK, MYL9, RAC2, RND3, PIP5K1C, |
| Actin-based | DIRAS3, ACTA2, RHOU |
| Motility by Rho | |
| Gαi Signaling | GNAI2, S1PR3, GNB4, RRAS2, RALA, |
| | LPAR1, CAV1, RGS4, GNG2, FPR1 |
| Epithelial Adherens | MYL9, TGFBR2, CDH2, NOTCH2, RRAS2, |
| Junction Signaling | TUBB6, SNAI2, ACTA2, TUBB2A, ACVR1, |
| | ZYX |
| Glioma Invasiveness | TIMP4, RRAS2, RND3, TIMP1, DIRAS3, |
| Signaling | RHOU |
| IGF-1 Signaling | IGFBP4, IGFBP6, NEDD4, CTGF, RRAS2, |
| | IGFBP5, IRS2, CYR61 |
| UDP-N-acetyl-D-glu- | UAP1, GFPT2 |
| cosamine | |
| Biosynthesis II | |
| Glycogen | UGP2, GBE1 |
| Biosynthesis II | |
| (from | |
| UDP-D-Glucose) | |
| Signaling by Rho | DIRAS3, ACTA2, ITGA5, CDH11, GNAI2, |
| Family GTPases | MYLK, MYL9, GNB4, CDH2, RND3, |
| | PIP5K1C, RHOU, GNG2, ARHGEF3 |
| NF-κB Signaling | IL33, TGFBR2, GHR, RRAS2, TNFRSF1A, |
| Complement System | TNFAIP3, MAP4K4, IL1R1, IRAK3, |
| | CARD11, TNFSF13B SERPING1, CD59, |
| | C1S, CFI |
| Colorectal Cancer | TGFBR2, GNB4, RRAS2, MMP23B, RND3, |
| Metastasis | TNFRSF1A, DIRAS3, RHOU, VEGFC, |
| Signaling | GNG2, PTGS2, PTGER2, LRP1, WNT5A |
| Remodeling of | RALA, TUBB6, ACTA2, TUBB2A, ZYX, |
| Epithelial Adherens | MAPRE3 |
| Junctions | |
| Sphingosine-1-phos- | GNAI2, S1PR3, RND3, DIRAS3, CASP1, |
| phate | RHOU, CASP4, SMPD1 |
| Signaling | |
| Virus Entry via | RAC2, RRAS2, ITSN1, FLNC, ACTA2, |
| Endocytic Pathways | CAV1, ITGA5 |
| CXCR4 Signaling | GNAI2, MYL9, GNB4, RRAS2, RND3, |
| | DIRAS3, CXCL12, RHOU, ITPR1, GNG2 |
| Semaphorin | RND3, DPYSL3, DPYSL4, DIRAS3, RHOU |
| Signaling in | |
| Neurons | |
| Role of | VCAM1, TNFRSF1A, FGF2, CXCL12, |
| Macrophages, | VEGFC, IRAK3, IL1R1, IL33, ROR2, |
| Fibroblasts and | TRAF3IP2, RRAS2, CCL2, DKK3, LRP1, |
| Endothelial | TNFSF13B, WNT5A |
| Cells in Rheumatoid | |
| Arthritis | |
| Dermatan Sulfate | CHSY3, CHPF, CSGALNACT1, HS3ST3A1, |
| Biosynthesis | DSE |
| Ephrin B Signaling | GNAI2, RAC2, GNB4, ITSN1, CXCL12, |
| Prostanoid Biosynthesis | GNG2 PTGIS, PTGS2 |
| Regulation of | RRAS2, ITGA5, TLN1, CAPN2, CAST |
| Cellular Mechanics | |
| by Calpain | |
| Protease | |
| Actin Nucleation by | RRAS2, RND3, DIRAS3, RHOU, ITGA5 |
| ARP-WASP Complex | |
| Axonal Guidance | SLIT3, RAC2, PAPPA, PLXNA3, ITSN1, |
| Signaling | TUBB2A, CXCL12, VEGFC, ITGA5, MYL9, |
| | GNAI2, GNB4, ADAMTS6, RRAS2, TUBB6, |
| | ABLIM3, RTN4, ADAM19, GNG2, BMP1, |
| | WNT5A |
| Thrombin Signaling | GNAI2, MYLK, MYL9, GNB4, RRAS2, |
| | RND3, DIRAS3, RHOU, ARHGEF3, ITPR1, |
| | GNG2 |
| Role of IL-17F in | TRAF3IP2, CCL2, RPS6KA2, CXCL6 |
| Allergic | |
| Inflammatory Airway | |
| Diseases | |
| Leukocyte | GNAI2, RAC2, CD99, TIMP4, VCAM1, |
| Extravasation | MMP23B, JAM3, TIMP1, ACTA2, CXCL12, |
| Signaling | THY1 |
| Cholecystokinin/ | IL33, RRAS2, RND3, DIRAS3, RHOU, |
| Gastrin-mediated | PTGS2, ITPR1 |
| Signaling | |
| Antiproliferative | GNB4, RRAS2, CDKN1A, GNG2, NPR2 |
| Role of Somato- | |
| statin Receptor 2 | |
| Chondroitin Sulfate | CHSY3, CHPF, CSGALNACT1, HS3ST3A1 |
| Biosynthesis (Late | |
| Stages) | |
| Ephrin Receptor | GNAI2, RAC2, GNB4, RRAS2, ITSN1, |
| Signaling | CXCL12, ITGA5, VEGFC, MAP4K4, GNG2 |
| FAK Signaling | RRAS2, ASAP1, ACTA2, ITGA5, TLN1, |
| | CAPN2 |
| Intrinsic | COL1A2, COL1A1, COL3A1 |
| Prothrombin | |
| Activation | |
| Pathway | |
| Gap Junction | GNAI2, RRAS2, TUBB6, LPAR1, ACTA2, |
| Signaling | TUBB2A, CAV1, ITPR1, NPR2 |
| Sulfate Activation | PAPSS2 |
| for Sulfonation | |
| Fatty Acid | ALDH1A3, PTGS2 |
| α-oxidation | |
| Chemokine | GNAI2, CCL13, RRAS2, CCL2, CXCL12 |
| Signaling | |
| PPAR Signaling | IL33, RRAS2, TNFRSF1A, MAP4K4, |
| | IL1R1, PTGS2 |
| Colanic Acid | UGP2, PMM1 |
| Building Blocks | |
| Biosynthesis | |
| LPS/IL-1 Mediated | IL33, ALDH1A3, TNFRSF1A, ACSL4, |
| Inhibition of RXR | IL1R1, ABCC3, PAPSS2, HS3ST3A1, |
| Function | ABCA1, GSTO1, MAOA |
| IL-6 Signaling | IL33, COL1A1, RRAS2, TNFRSF1A, |
| | MAP4K4, IL1R1, TNFAIP6 |
| Caveolar-mediated | ITSN1, FLNC, ACTA2, CAV1, ITGA5 |
| Endocytosis Signaling | |
| IL-17 Signaling | TRAF3IP2, RRAS2, CCL2, TIMP1, PTGS2 |
| Chondroitin Sulfate | CHSY3, CHPF, CSGALNACT1, HS3ST3A1 |
| Biosynthesis | |
| Triacylglycerol | PPAPDC1A, GPAM, PPAP2A |
| Biosynthesis | |
| Breast Cancer | GNAI2, GNB4, RRAS2, TUBB6, PPP1R3C, |
| Regulation by | CDKN1A, TUBB2A, ARHGEF3, ITPR1, |
| Stathmin1 | GNG2 |
| Tryptophan | ALDH1A3, MAOA |
| Degradation X | |
| (Mammalian, via | |
| Tryptamine) | |
| Putrescine | ALDH1A3, MAOA |
| Degradation III | |
| Glutathione Redox | GLRX |
| Reactions II | |
| Atherosclerosis | IL33, COL1A2, COL1A1, VCAM1, CCL2, |
| Signaling | CXCL12, COL3A1 |
| Toll-like Receptor | TICAM2, TNFAIP3, MAP4K4, IRAK3 |
| Signaling | |
| γ-linolenate | CYB5R3, ACSL4 |
| Biosynthesis II | |
| (Animals) | |
| Coagulation System | SERPINE1, BDKRB1, PLAT |
| Phospholipase C | MYL9, GNB4, RRAS2, RALA, RND3, |
| Signaling | DIRAS3, RHOU, ITGA5, ARHGEF3, ITPR1, |
| | GNG2 |
| Arsenate | GSTO1 |
| Detoxification I | |
| (Glutaredoxin) | |
| Melatonin | MAOA |
| Degradation II | |
| Molecular | RAC2, RALA, APH1B, DIRAS3, HIF1A, |
| Mechanisms of | TGFBR2, GNAI2, RRAS2, RND3, CDKN1A, |
| Cancer | RHOU, ARHGEF3, LRP1, BMP1, WNT5A |
| fMLP Signaling in | GNAI2, GNB4, RRAS2, ITPR1, GNG2, |
| Neutrophils | FPR1 |
| TR/RXR Activation | KLF9, COL6A3, SLC16A2, HIF1A, RCAN2 |
| α-Adrenergic | GNAI2, GNB4, RRAS2, ITPR1, GNG2 |
| Signaling | |
| Actin Cytoskeleton | MYLK, MYL9, RAC2, RRAS2, PIP5K1C, |
| Signaling | FGF2, ACTA2, ITGA5, TLN1, ARHGAP24 |
| Dopamine | ALDH1A3, MAOA |
| Degradation | |
| Bladder Cancer | MMP23B, RRAS2, FGF2, CDKN1A, VEGFC |
| Signaling | |
| Pyruvate | LDHA |
| Fermentation to | |
| Lactate | |
| Tetrapyrrole | ALAD |
| Biosynthesis II | |
| Glycerol | GK |
| Degradation I | |
| Purine | PGM2 |
| Ribonucleosides | |
| Degradation to | |
| Ribose-1-phosphate | |
| Tyrosine | FAH |
| Degradation I | |
| G Beta Gamma | GNAI2, GNB4, RRAS2, CAV1, GNG2 |
| Signaling | |
| CCR3 Signaling in | GNAI2, MYLK, GNB4, RRAS2, ITPR1, |
| Eosinophils | GNG2 |
| PPARα/RXRα | TGFBR2, GHR, RRAS2, ACVR1, MAP4K4, |
| Activation | IL1R1, GK, ABCA1 |
| RhoA Signaling | MYLK, MYL9, LPAR1, RND3, PIP5K1C, |
| | ACTA2 |
| Agrin Interactions | RAC2, RRAS2, ACTA2, ITGA5 |
| at Neuromuscular | |
| Junction | |
| IL-1 Signaling | GNAI2, GNB4, IL1R1, IRAK3, GNG2 |
| p38 MAPK Signaling | IL33, TGFBR2, TNFRSF1A, IL1R1, |
| | RPS6KA2, IRAK3 |
| Cardiac Hypertrophy | GNAI2, MYL9, TGFBR2, GNB4, HAND2, |
| Signaling | RRAS2, RND3, DIRAS3, RHOU, GNG2 |
| Acute Phase | IL33, SERPING1, RRAS2, TNFRSF1A, |
| Response | C1S, OSMR, IL1R1, SERPINE1 |
| Signaling | |
| MSP-RON Signaling | CCL2, ACTA2, MST1 |
| Pathway | |
| Thioredoxin | TXNRD1 |
| Pathway | |
| GDP-glucose | PGM2 |
| Biosynthesis | |
| GDP-mannose | PMM1 |
| Biosynthesis | |
| GDNF Family | RRAS2, DOK4, IRS2, ITPR1 |
| Ligand-Receptor | |
| Interactions | |
| PTEN Signaling | TGFBR2, RAC2, GHR, RRAS2, CDKN1A, |
| | ITGA5 |
| Glioblastoma | RRAS2, RND3, DIRAS3, CDKN1A, RHOU, |
| Multiforme | ITPR1, WNT5A |
| Signaling | |
| Gαq Signaling | GNB4, RND3, DIRAS3, RHOU, RGS4, |
| | ITPR1, GNG2 |
| LXR/RXR Activation | IL33, CCL2, TNFRSF1A, IL1R1, PTGS2, |
| | ABCA1 |
| Glucose and | PGM2 |
| Glucose-1-phosphate | |
| Degradation | |
| Sphingomyelin | SMPD1 |
| Metabolism | |
| Sertoli | RRAS2, TUBB6, JAM3, TNFRSF1A, ACTA2, |
| Cell-Sertoli Cell | TUBB2A, ITGA5, RAB8B |
| Junction Signaling | |
| Role of NFAT in | GNAI2, TGFBR2, RCAN1, GNB4, RRAS2, |
| Cardiac | ITPR1, GNG2, RCAN2 |
| Hypertrophy | |
| Paxillin Signaling | PARVA, RRAS2, ACTA2, ITGA5, TLN1 |
| Glucocorticoid | TGFBR2, VCAM1, CCL13, RRAS2, CCL2, |
| Receptor | SMARCA2, SGK1, CDKN1A, PTGS2, |
| Signaling | SERPINE1, FKBP5 |
| Serotonin | ALDH1A3, CSGALNACT1, MAOA |
| Degradation | |
| HIF1α Signaling | MMP23B, RRAS2, VEGFC, HIF1A, LDHA |
| Clathrin-mediated | SH3BP4, PIP5K1C, FGF2, ACTA2, ITGA5, |
| Endocytosis | DAB2, VEGFC, SH3KBP1 |
| Signaling | |
| mTOR Signaling | RRAS2, RND3, DIRAS3, RHOU, VEGFC, |
| | HIF1A, RPS6KA2, RPS27L |
| VDR/RXR Activation | IGFBP6, WT1, CDKN1A, IGFBP5 |
| Ceramide Signaling | S1PR3, RRAS2, TNFRSF1A, SMPD1 |
| Role of IL-17A in | CCL2, PTGS2, CXCL6 |
| Arthritis | |
| Calcium Transport I | ATP2B4 |
| Heme | ALAD |
| Biosynthesis II | |
| UDP-N-acetyl-D- | UAP1 |
| galactosamine | |
| Biosynthesis II | |
| Pancreatic | TGFBR2, RALA, CDKN1A, VEGFC, PTGS2 |
| Adenocarcinoma | |
| Signaling | |
| TREM1 | CCL2, CASP1, ITGA5 |
| Signaling | |
| G Protein Signaling | GNB4, GNG2 |
| Mediated by Tubby | |
| Role of Tissue | CTGF, RRAS2, VEGFC, RPS6KA2, CYR61 |
| Factor in Cancer | |
| Human Embryonic | S1PR3, TGFBR2, FGF2, ACVR1, WNT5A, |
| Stem Cell | BMP1 |
| Pluripotency | |
| Role of Osteoblasts, | IL33, COL1A1, DKK3, TNFRSF1A, ITGA5, |
| Osteoclasts and | IL1R1, LRP1, WNT5A, BMP1 |
| Chondrocytes | |
| in Rheumatoid | |
| Arthritis | |
| Role of JAK2 in | GHR, IRS2 |
| Hormone-like | |
| Cytokine Signaling | |
| Noradrenaline and | ALDH1A3, MAOA |
| Adrenaline | |
| Degradation | |
| Glycogen | PGM2 |
| Degradation II | |
| TGF-β Signaling | TGFBR2, RRAS2, ACVR1, SERPINE1 |
| Circadian Rhythm | ARNTL, BHLHE40 |
| Signaling | |
| Role of NFAT in | GNAI2, RCAN1, GNB4, RRAS2, ITPR1, |
| Regulation of the | GNG2, RCAN2 |
| Immune Response | |
| Oncostatin M | RRAS2, OSMR |
| Signaling | |
| Neuregulin | RRAS2, DCN, ITGA5, ERRFI1 |
| Signaling | |
| Endothelin-1 | GNAI2, RRAS2, CASP1, CASP4, PTGER2, |
| Signaling | PTGS2, ITPR1 |
| Role of JAK1 and | IL7R, RRAS2, IRS2 |
| JAK3 in γc | |
| Cytokine Signaling | |
| Factors Promoting | TGFBR2, ACVR1, LRP1, BMP1 |
| Cardiogenesis in | |
| Vertebrates | |
| IL-17A Signaling in | TRAF3IP2, CCL2 |
| Fibroblasts | |
| Eicosanoid | PTGIS, PTGER2, PTGS2 |
| Signaling | |
| PAK Signaling | MYLK, MYL9, RRAS2, ITGA5 |
| Apoptosis | RRAS2, TNFRSF1A, MAP4K4, CAPN2 |
| Signaling | |
| Calcium Signaling | MYL9, RCAN1, ACTA2, TPM2, ITPR1, |
| | RCAN2, ATP2B4 |
| Glycogen | PGM2 |
| Degradation III | |
| Histamine | ALDH1A3 |
| Degradation | |
| Guanosine | AOX1 |
| Nucleotides | |
| Degradation III | |
| VEGF Signaling | RRAS2, ACTA2, VEGFC, HIF1A |
| Gα12/13 | MYL9, CDH2, RRAS2, LPAR1, CDH11 |
| Signaling | |
| ERK5 Signaling | RRAS2, SGK1, RPS6KA2 |
| Notch Signaling | NOTCH2, APH1B |
| Role of IL-17A in | CXCL6 |
| Psoriasis | |
| Fatty Acid | ACSL4 |
| Activation | |
| Urate | AOX1 |
| Biosynthesis/Inosine | |
| 5′-phosphate | |
| Degradation | |
| NRF2-mediated | RRAS2, ACTA2, HSPB8, AOX1, FKBP5, |
| Oxidative | TXNRD1, GSTO1 |
| Stress Response | |
| p53 Signaling | WT1, SNAI2, CDKN1A, SERPINE2 |
| Fcγ Receptor- | MYO5A, RAC2, ACTA2, TLN1 |
| mediated | |
| Phagocytosis in | |
| Macrophages and | |
| Monocytes | |
| SAPK/JNK Signaling | RAC2, RRAS2, MAP4K4, GNG2 |
| Netrin Signaling | RAC2, ABLIM3 |
| Role of MAPK | RRAS2, CCL2, PTGS2 |
| Signaling in | |
| the Pathogenesis of | |
| Influenza | |
| Aldosterone | NEDD4, PIP5K1C, SGK1, HSPB8, ITPR1, |
| Signaling in | HSPB6 |
| Epithelial Cells | |
| Phenylalanine | MAOA |
| Degradation IV | |
| (Mammalian, via | |
| Side Chain) | |
| Adenosine | AOX1 |
| Nucleotides | |
| Degradation II | |
| CCR5 Signaling in | GNAI2, GNB4, GNG2 |
| Macrophages | |
| Tight Junction | MYLK, MYL9, TGFBR2, JAM3, TNFRSF1A, |
| Signaling | ACTA2 |
| Mechanisms of Viral | NEDD4, ACTA2 |
| Exit from Host | |
| Cells | |
| IL-10 Signaling | IL33, MAP4K4, IL1R1 |
| eNOS Signaling | LPAR1, CAV1, VEGFC, ITPR1, BDKRB1 |
| Role of | |
| Hypercytokinemia/ | IL33, CCL2 |
| hyperchemokinemia | |
| in the | |
| Pathogenesis of | |
| Influenza | |
| Dermatan Sulfate | HS3ST3A1, DSE |
| Biosynthesis | |
| (Late Stages) | |
| CDP-diacylglycerol | GPAM |
| Biosynthesis I | |
| Oxidative Ethanol | ALDH1A3 |
| Degradation III | |
| Melanoma Signaling | RRAS2, CDKN1A |
| Huntington's | GNB4, SGK1, CASP1, CASP4, CAPN2, |
| Disease Signaling | ITPR1, GNG2, SNAP25 |
| cAMP-mediated | GNAI2, S1PR3, GPER, PDE7B, LPAR1, |
| signaling | RGS4, PTGER2, FPR1 |
| Nicotine | CSGALNACT1, AOX1 |
| Degradation III | |
| Phosphatidylglycerol | GPAM |
| Biosynthesis II | |
| (Non-plastidic) | |
| Purine Nucleotides | AOX1 |
| Degradation II | |
| (Aerobic) | |
| Mitochondrial | ACSL4 |
| L-carnitine | |
| Shuttle Pathway | |
| Ethanol | ALDH1A3 |
| Degradation IV | |
| Nitric Oxide | CAV1, VEGFC, ITPR1 |
| Signaling in the | |
| Cardiovascular | |
| System | |
| Relaxin Signaling | GNAI2, GNB4, PDE7B, GNG2, NPR2 |
| G-Protein Coupled | GNAI2, S1PR3, GPER, RRAS2, PDE7B, |
| Receptor Signaling | LPAR1, RGS4, PTGER2, FPR1 |
| Differential | CCL2 |
| Regulation of | |
| Cytokine Production | |
| in Macrophages and | |
| T Helper Cells by | |
| IL-17A and IL-17F | |
| Hepatic Cholestasis | IL33, TNFRSF1A, IL1R1, IRAK3, ABCC3 |
| TNFR1 Signaling | TNFRSF1A, TNFAIP3 |
| Wnt/β-catenin | TGFBR2, CDH2, DKK3, ACVR1, LRP1, |
| Signaling | WNT5A |
| Lipid Antigen | CD1D |
| Presentation | |
| by CD1 | |
| GADD45 Signaling | CDKN1A |
| Regulation of IL-2 | TGFBR2, RRAS2, CARD11 |
| Expression in | |
| Activated and | |
| Anergic T | |
| Lymphocytes | |
| Renin-Angiotensin | RRAS2, CCL2, PTGER2, ITPR1 |
| Signaling | |
| Gas Signaling | GNB4, GPER, PTGER2, GNG2 |
| Corticotropin | GNAI2, PTGS2, ITPR1, NPR2 |
| Releasing | |
| Hormone Signaling | |
| CNTF Signaling | RRAS2, RPS6KA2 |
| Dendritic Cell | IL33, COL1A2, COL1A1, TNFRSF1A, |
| Maturation | COL3A1, CD1D |
| Androgen Signaling | GNAI2, GNB4, TGFB1I1, GNG2 |
| Nicotine | CSGALNACT1, AOX1 |
| Degradation II | |
| Amyloid Processing | APH1B, CAPN2 |
| Superpathway of | CSGALNACT1, MAOA |
| Melatonin | |
| Degradation | |
| Type II Diabetes | TNFRSF1A, ACSL4, IRS2, SMPD1 |
| Mellitus Signaling | |
| Glycolysis I | ENO2 |
| 14-3-3-mediated | RRAS2, TUBB6, TNFRSF1A, TUBB2A |
| Signaling | |
| Protein Kinase A | GNAI2, MYLK, MYL9, TGFBR2, GNB4, |
| Signaling | PDE7B, FLNC, PPP1R3C, KDELR3, PTGS2, |
| | ITPR1, GNG2 |
| Differential | CCL2 |
| Regulation of | |
| Cytokine Production | |
| in Intestinal | |
| Epithelial Cells by | |
| IL-17A and IL-17F | |
| Glutathione-mediated | GSTO1 |
| Detoxification | |
| Gluconeogenesis I | ENO2 |
| P2Y Purigenic | GNAI2, GNB4, RRAS2, GNG2 |
| Receptor Signaling | |
| Pathway | |
| Thrombopoietin | RRAS2, IRS2 |
| Signaling | |
| Tumoricidal | SRGN |
| Function of | |
| Hepatic Natural | |
| Killer Cells | |
| Estrogen-mediated | CDKN1A |
| S-phase Entry | |
| Thyroid Hormone | CSGALNACT1 |
| Metabolism II (via | |
| Conjugation and/or | |
| Degradation) | |
| PI3K/AKT Signaling | RRAS2, CDKN1A, ITGA5, PTGS2 |
| Role of JAK family | OSMR |
| kinases in | |
| IL-6-type Cytokine | |
| Signaling | |
| Calcium-induced T | CAPN2, ITPR1 |
| Lymphocyte | |
| Apoptosis | |
| ErbB4 Signaling | RRAS2, APH1B |
| Death Receptor | TNFRSF1A, MAP4K4 |
| Signaling | |
| ATM Signaling | CDKN1A, TDP1 |
| Antiproliferative | TGFBR2 |
| Role of TOB in T | |
| Cell Signaling | |
| D-myo-inositol | PIP5K1C |
| (1,4,5)-Trisphos- | |
| phate | |
| Biosynthesis | |
| Chronic Myeloid | TGFBR2, RRAS2, CDKN1A |
| Leukemia Signaling | |
| Role of BRCA1 in | SMARCA2, CDKN1A |
| DNA Damage | |
| Response | |
| B Cell Development | IL7R |
| TNFR2 Signaling | TNFAIP3 |
| Retinoate | ALDH1A3 |
| Biosynthesis I | |
| Fatty Acid | ACSL4 |
| β-oxidation I | |
| Ethanol | ALDH1A3 |
| Degradation II | |

TABLE 6
PATHWAYS UPREGULATED IN CLUSTER 2

| Ingenuity Canonical | |
|---|---|
| Pathways | Molecules |
| Cell Cycle Control | MCM3, MCM6, MCM2, CDT1, CDK4, |
| of Chromosomal | ORC6, MCM4, CDK2, MCM7 |
| Replication | |
| Estrogen-mediated | CCNE1, CDK4, E2F5, CDK1, E2F2, |
| S-phase Entry | CDK2 |
| Cell Cycle | PRMT1, CCNE1, CDK4, E2F5, E2F2, |
| Regulation by | CDK2 |
| BTG Family Proteins | |
| Role of BRCA1 in | FANCD2, FANCG, E2F5, RBBP8, RFC5, |
| DNA Damage Response | E2F2, RFC3 |
| Cyclins and Cell | CCNE1, CDK4, WEE1, E2F5, CDK1, |
| Cycle Regulation | E2F2, CDK2 |
| Role of CHK Proteins | E2F5, RFC5, CDK1, E2F2, CDK2, |
| in Cell Cycle | RFC3 |
| Checkpoint Control | |
| GADD45 Signaling | CCNE1, CDK4, CDK1, CDK2 |
| Superpathway of | ACAT2, TM7SF2, HMGCS1, LBR |
| Cholesterol | |
| Biosynthesis | |
| Hereditary Breast | FANCD2, FANCG, CDK4, WEE1, RFC5, |
| Cancer Signaling | CDK1, RFC3 |
| Myo-inositol | ISYNA1, IMPA2 |
| Biosynthesis | |
| Cell Cycle: G1/S | CCNE1, CDK4, E2F5, E2F2, CDK2 |
| Checkpoint | |
| Regulation | |
| Pyridoxal | NEK2, CDK4, TTK, CDK1, CDK2 |
| 5′-phosphate | |
| Salvage Pathway | |
| dTMP De Novo | TYMS, SHMT1 |
| Biosynthesis | |
| DNA damage-induced | CCNE1, CDK1, CDK2 |
| 14-3-3σ | |
| Signaling | |
| Zymosterol | TM7SF2, LBR |
| Biosynthesis | |
| Cell Cycle: G2/M | WEE1, CKS1B, TOP2A, CDK1 |
| DNA Damage | |
| Checkpoint | |
| Regulation | |
| Glioblastoma | CCNE1, PLCG2, CDK4, FZD3, E2F5, |
| Multiforme | E2F2, CDK2 |
| Signaling | |
| Breast Cancer | STMN1, CCNE1, ARHGEF16, E2F5, |
| Regulation by | PPP1R14A, CDK1, E2F2, CDK2 |
| Stathmin1 | |
| Salvage Pathways of | NEK2, CDK4, TTK, CDK1, CDK2 |
| Pyrimidine | |
| Ribonucleotides | |
| Folate | MTHFD2, SHMT1 |
| Transformations I | |
| Regulation of | CCNE1, CDK4, CDK1, CDK2 |
| Cellular | |
| Mechanics by Calpain | |
| Protease | |
| Glutaryl-CoA | ACAT2, HSD17B8 |
| Degradation | |
| Ketogenesis | ACAT2, HMGCS1 |
| ATM Signaling | FANCD2, CBX5, CDK1, CDK2 |
| Mevalonate Pathway I | ACAT2, HMGCS1 |
| Cholesterol | TM7SF2, LBR |
| Biosynthesis I | |
| Cholesterol | TM7SF2, LBR |
| Biosynthesis II (via | |
| 24,25-dihydrolano- | |
| sterol) | |
| Cholesterol | TM7SF2, LBR |
| Biosynthesis III | |
| (via Desmosterol) | |
| Notch Signaling | JAG2, DLL3, HEY1 |
| Pancreatic | CCNE1, CDK4, E2F5, E2F2, CDK2 |
| Adenocarcinoma | |
| Signaling | |
| Small Cell Lung | CCNE1, CDK4, CKS1B, CDK2 |
| Cancer Signaling | |
| Granzyme B Signaling | LMNB1, PARP1 |
| Mismatch Repair in | RFC5, RFC3 |
| Eukaryotes | |
| Superpathway of | ACAT2, HMGCS1 |
| Geranylgeranyl- | |
| diphosphate | |
| Biosynthesis I (via | |
| Mevalonate) | |
| Tryptophan | ACAT2, HSD17B8 |
| Degradation III | |
| (Eukaryotic) | |
| Glycine | SHMT1 |
| Biosynthesis I | |
| Glutamate Dependent | GAD1 |
| Acid Resistance | |
| Tyrosine | PCBD1 |
| Biosynthesis IV | |
| Fatty Acid | ECI1 |
| β-oxidation III | |
| (Unsaturated, Odd | |
| Number) | |
| Glioma Signaling | PLCG2, CDK4, E2F5, E2F2 |
| Antiproliferative Role | CCNE1, CDK2 |
| of TOB in T Cell | |
| Signaling | |
| Mitotic Roles of | PLK4, WEE1, CDK1 |
| Polo-Like Kinase | |
| Creatine-phosphate | CKB |
| Biosynthesis | |
| Methylmalonyl Pathway | PCCB |
| Phenylalanine | PCBD1 |
| Degradation I | |
| (Aerobic) | |
| Fatty Acid | ECI1, HSD17B8 |
| β-oxidation I | |
| Superpathway of | PRMT1, PCCB |
| Methionine Degradation | |
| Tetrahydrofolate | MTHFD2 |
| Salvage from 5,10- | |
| methenyltetrahydro- | |
| folate | |
| 2-oxobutanoate | PCCB |
| Degradation I | |
| Glutamate Degradation | GAD1 |
| III (via | |
| 4-aminobutyrate) | |
| Folate | SHMT1 |
| Polyglutamylation | |
| NAD Biosynthesis from | QPRT |
| 2-amino-3- | |
| carboxymuconate | |
| Semialdehyde | |
| Superpathway of Serine | SHMT1 |
| and Glycine | |
| Biosynthesis I | |
| Pentose Phosphate | RPIA |
| Pathway | |
| (Non-oxidative Branch) | |
| Glycine Cleavage | GLDC |
| Complex | |
| Selenocysteine | SARS2 |
| Biosynthesis II | |
| (Archaea and | |
| Eukaryotes) | |
| Salvage Pathways of | TK1 |
| Pyrimidine | |
| Deoxyribonucleotides | |
| Molecular Mechanisms | CCNE1, FANCD2, CDK4, ARHGEF16, |
| of Cancer | FZD3, E2F5, E2F2, CDK2 |
| Phosphatidylcholine | CHKA |
| Biosynthesis I | |
| Phosphatidylethanolamine | CHKA |
| Biosynthesis II | |
| Histidine Degradation | MTHFD2 |
| III | |
| Ketolysis | ACAT2 |
| Aryl Hydrocarbon | CCNE1, CDK4, CDK2, MCM7 |
| Receptor Signaling | |
| Factors Promoting | CCNE1, FZD3, CDK2 |
| Cardiogenesis in | |
| Vertebrates | |
| Apoptosis Signaling | PLCG2, CDK1, PARP1 |
| Pentose Phosphate | RPIA |
| Pathway | |
| Glycine Betaine | SHMT1 |
| Degradation | |
| Chronic Myeloid | CDK4, E2F5, E2F2 |
| Leukemia Signaling | |
| HGF Signaling | ELF3, PLCG2, CDK2 |
| Tight Junction | CDK4, CGN, CNKSR3, PARD6A |
| Signaling | |
| NAD biosynthesis II | QPRT |
| (from tryptophan) | |
| DNA Double-Strand Break | PARP1 |
| Repair by | |
| Non-Homologous End | |
| Joining | |
| Isoleucine | ACAT2 |
| Degradation I | |
| Pyrimidine | TYMS |
| Deoxyribonucleotides | |
| De Novo Biosynthesis I | |
| Methionine | PRMT1 |
| Degradation I (to | |
| Homocysteine) | |
| Extrinsic Prothrombin | F12 |
| Activation Pathway | |
| Glutathione Redox | GPX4 |
| Reactions I | |
| Granzyme A Signaling | HMGB2 |
| Cysteine Biosynthesis | PRMT1 |
| III (mammalia) | |
| D-myo-inositol | IMPA2 |
| (1,4,5)-trisphosphate | |
| Degradation | |
| Dopamine Receptor | PPP1R14A, PCBD1 |
| Signaling | |
| Mitochondrial | UCP2, CYC1, GPX4 |
| Dysfunction | |
| HER-2 Signaling in | CCNE1, PARD6A |
| Breast Cancer | |
| Superpathway of | IMPA2 |
| D-myo-inositol | |
| (1,4,5)-trisphosphate | |
| Metabolism | |
| Reelin Signaling in | ARHGEF16, PAFAH1B3 |
| Neurons | |
| Prostate Cancer | CCNE1, CDK2 |
| Signaling | |
| D-myo-inositol | PLCG2 |
| (1,4,5)-Trisphosphate | |
| Biosynthesis | |
| Systemic Lupus | LSM2, SNRPB, PLCG2, SNRPF |
| Erythematosus | |
| Signaling | |
| Intrinsic Prothrombin | F12 |
| Activation Pathway | |
| FXR/RXR Activation | SDC1, FGFR4 |
| TR/RXR Activation | UCP2, STRBP |
| Sonic Hedgehog | CDK1 |
| Signaling | |
| G Protein Signaling | PLCG2 |
| Mediated by Tubby | |
| Serotonin Receptor | PCBD1 |
| Signaling | |
| p53 Signaling | CDK4, CDK2 |
| Inhibition of | SDC1 |
| Angiogenesis by TSP1 | |
| tRNA Splicing | PDE9A |
| Coagulation System | F12 |
| Estrogen Biosynthesis | HSD17B8 |
| Wnt/β-catenin | SOX4, FZD3, BCL9 |
| Signaling | |
| tRNA Charging | SARS2 |
| Inhibition of Matrix | SDC1 |
| Metalloproteases | |
| Netrin Signaling | ABLIM1 |
| Mechanisms of Viral | LMNB1 |
| Exit from Host Cells | |
| Transcriptional | FOXC1 |
| Regulatory Network in | |
| Embryonic Stem Cells | |
| FcγRIIB Signaling in B | PLCG2 |
| Lymphocytes | |
| Melanoma Signaling | CDK4 |
| Role of Oct4 in | PARP1 |
| Mammalian Embryonic | |
| Stem Cell | |
| Pluripotency | |
| MSP-RON Signaling | F12 |
| Pathway | |
| ERK/MAPK Signaling | ELF3, PLCG2, PPP1R14A |
| Primary | UNG |
| Immunodeficiency | |
| Signaling | |
| GABA Receptor | GAD1 |
| Signaling | |
| Synaptic Long Term | PLCG2, PPP1R14A |
| Potentiation | |
| PTEN Signaling | FGFR4, CNKSR3 |
| Ephrin A Signaling | EFNA1 |
| CD27 Signaling in | SIVA1 |
| Lymphocytes | |
| Semaphorin Signaling | SEMA4D |
| in Neurons | |
| D-myo-inositol-5-phos- | PLCG2, PPP1R14A |
| phate Metabolism | |
| TREM1 Signaling | PLCG2 |
| Thrombopoietin | PLCG2 |
| Signaling | |
| Phospholipases | PLCG2 |
| ErbB2-ErbB3 Signaling | ETV4 |
| Ovarian Cancer | CDK4, FZD3 |
| Signaling | |
| Cardiac | PDE9A, PPP1R14A |
| β-adrenergic | |
| Signaling | |
| ErbB4 Signaling | PLCG2 |
| Human Embryonic Stem | FGFR4, FZD3 |
| Cell Pluripotency | |
| Retinoic acid Mediated | PARP1 |
| Apoptosis Signaling | |
| Estrogen-Dependent | HSD17B8 |
| Breast Cancer | |
| Signaling | |
| Hypoxia Signaling in | UBE2C |
| the Cardiovascular | |
| System | |
| Angiopoietin Signaling | GRB14 |
| Non-Small Cell Lung | CDK4 |
| Cancer Signaling | |
| Erythropoietin | PLCG2 |
| Signaling | |
| CCR5 Signaling in | PLCG2 |
| Macrophages | |
| Melatonin Signaling | PLCG2 |
| Growth Hormone | PLCG2 |
| Signaling | |
| GDNF Family | PLCG2 |
| Ligand-Receptor | |
| Interactions | |
| Chemokine Signaling | PLCG2 |
| Macropinocytosis | PLCG2 |
| Signaling | |
| Basal Cell Carcinoma | FZD3 |
| Signaling | |

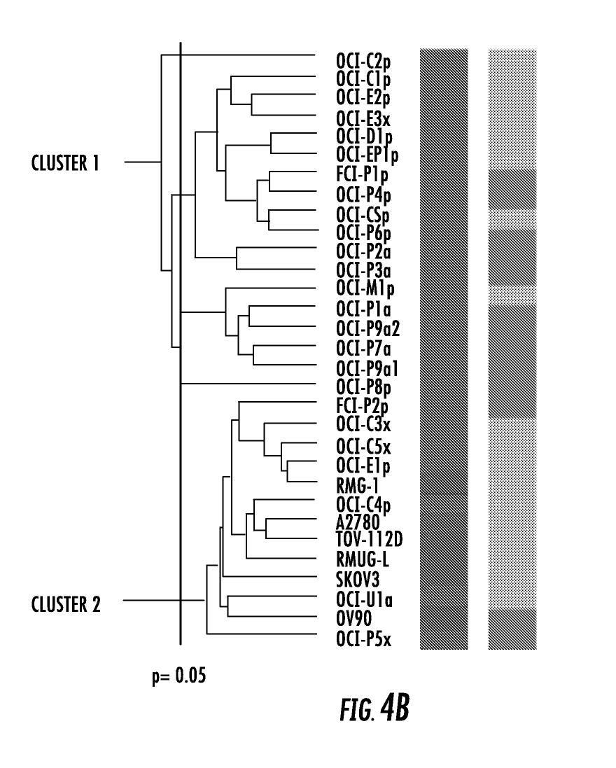

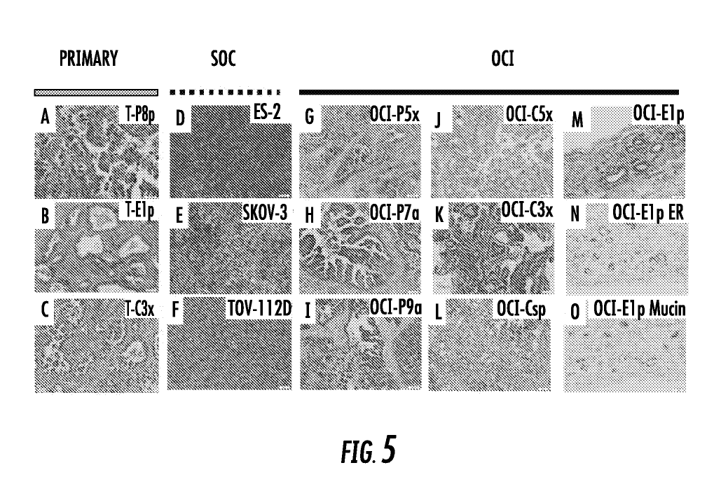

OCI cell lines reproduce human tumor histopathology as mouse xenografts. To examine the in vivo tumor phenotype of the OCI lines, we injected each into immunocompromised mouse hosts. The microscopic features of tumors have long been used to describe ovarian tumor subtypes. The most common malignant ovarian tumor subtype, papillary serous carcinoma, displays finger-like structures (papillae) that consist of central stromal cores giving rise to smaller branches lined by a malignant epithelium with minimal cytoplasm and very large, high grade, round nuclei (FIG. 5a). Endometrioid adenocarcinoma named for its similarity to endometrial tissue, features glands organized around central lumina surrounded by elongated malignant epithelial cells with abundant cytoplasm (FIG. 5b). Clear cell carcinoma typically forms back-to-back micro-cysts, glands and/or papillae that are lined with cells with abundant cytoplasm that appears clear in H&E stains due to excess cytoplasmic glycogen (FIG. 5c). Instead of glycogens, mucinous cancers have high levels of mucin in their cytoplasm. These differences in tumor morphology reflect relevant differences in gene expression and clinical features. However, recapitulating architectural features of primary tumors has been an elusive goal in most xenograft tumor models.

The SOC lines generally produce poorly differentiated xenograft tumors in mice without distinctive histopathologic features of specific ovarian tumor subtypes (FIG. 5 d-f). In contrast, the OCI lines produced tumor xenografts with a histopathology strongly resembling the original human tumor (FIG. 5 g-o). OCI-P5x, P7a and P9a were established from human papillary serous carcinoma, and they recapitulated the papillary serous-like specific architecture in immunocompromised mice (FIG. 5, g-i). The OCI-C3x and C5x lines were established from human clear cell, and they formed microcysts and papillae lined by clear cells in mice (FIGS. 5, j and k). The OCI-CSp line was established from a poorly differentiated carcinosarcoma and it formed a poorly differentiated tumor in mice (FIG. 5 l). The OCI-Elp line was established from an endometrioid adenocarcinoma and formed estrogen receptor positive tumors with a glandular architecture, recapitulating the original tumor phenotype (FIG. 5, m-o).

In summary, quite remarkably, the OCI lines formed tumors that were morphologic phenocopies of corresponding human ovarian carcinomas at the histopathologic level, unlike SOC lines, which generally lack this characteristic (FIG. 5 d-f).

mRNA profile of OCI lines identify human tumors with different outcomes. Clustering analysis of the OCI and SOC cell line panel together with 285 human ovarian tumor specimens revealed two distinct patient clusters. Patient Cluster P1 included only OCI lines, and Cluster P2 included all the SOC lines (FIG. 1). The distribution of the cell lines within human tumor samples was identical to the in vitro cell line clusters, with the exception of a single cell line (OCI-C4p), indicating that the in vitro phenotype of these cell lines conform to in vivo clinical tumor phenotypes. Furthermore, the comparison of the clinical outcomes of these two groups of patients revealed that the patients with OCI-like tumors in Cluster 1 had a shorter progression-free and overall survival than patients in Cluster 2 with a SOC-like profile (FIG. 1).

In vitro Taxol response of OCI lines correlate with patient outcome. The striking correlation between poor patient outcomes and OCI lines in mRNA/RPPA Cluster 1 prompted us to test the response of these cell lines to Taxol and Cisplatin, which are two of the most commonly used drugs for the treatment of ovarian cancer. We selected a panel of lines that correspond to the OCI lines in mRNA/RPPA Clusters 1 and 2 and the SOC lines in mRNA/RPPA Cluster 2; each panel included examples of different tumor subtypes (P, C, CS, E, M), and tissue sources (solid tumors, ascites fluid, and xenograft explants). Both OCI and SOC lines were plated in WIT-OC medium for the above experiments. In these experiments we observed that the OCI lines in mRNA/RPPA Cluster 1 were less sensitive to Taxol than SOC lines in mRNA/RPPA Cluster 2 (FIG. 2). The subset of OCI lines in Cluster 2, similar to SOC lines in the same cluster, was also more sensitive to Taxol compared to OCI lines in Cluster 1 (FIG. 2). These results were confirmed with a full dose-response curve. In contrast, we did not find a significant difference in the response to Cisplatin between OCI and SOC lines.

To explore the possible basis for the relative Taxol resistance of OCI cells, we compared the protein profiles of Cluster1/OCI lines with Cluster2/SOC lines (FIG. 2, Table 7). Cluster 1 drug-resistant lines over-expressed several proteins that have been previously associated with Taxol resistance including Tubulin (the target of Taxol), PAX2, Cox2, PAI.1, AKT, PTEN, SMAD3 and activated Erk (MAPKpT202). Cluster 2 drug-sensitive cell lines displayed higher levels of several pro-apoptotic proteins, e.g. Bim, SMAC-DIABLO, and cleaved caspase 7, and lower levels of inactive phosphorylated BAD (pS112), a BH3-only pro-apoptotic protein; these proteins could render Cluster 2 cells more sensitive to Taxol-induced apoptosis. High Bim levels were anti-correlated with activated Erk (MAPKpT202), which phosphorylates Bim and targets it for ubiqutination and degradation (Table 2). These results suggest that OCI cell lines may be a valuable addition to the existing SOC cell lines for preclinical studies of ovarian cancer drug response.

TABLE 7
Protein profiles of Cluster1/OCI lines with Cluster2/SOC lines

| | Mean | Mean | std dev | sted dev | | |
|---|---|---|---|---|---|---|
| | Cluster 1 | Cluster 2 | Cluster 1 | Cluster 2 | | |
| Label | (OCI) | (OCI + SOC) | (OCI) | (OCI + SOC) | fold change | p-value |
| PAX2 | 0.936673031 | 0.592255571 | 0.060566718 | 0.036848886 | 1.581535198 | 3.95E−09 |
| PAI.1 | 9.111794132 | 0.787009925 | 2.22850434 | 1.190401543 | 11.57773726 | 3.41E−05 |
| Collagen.VI | 1.257114205 | 0.913938863 | 0.126287946 | 0.097700332 | 1.37549048 | 5.05E−05 |
| ab_Crystalline | 8.560730437 | 4.741980821 | 1.61070263 | 0.85144729 | 1.805306846 | 5.88E−05 |
| ACC_pS79 | 0.140629123 | 0.319996596 | 0.038849229 | 0.101743744 | 0.439470684 | 0.000260295 |
| p90RSK | 0.228736327 | 0.409342638 | 0.016120081 | 0.116487022 | 0.5587894 | 0.000329342 |
| N.Cadherin | 0.147774037 | 0.115515317 | 0.009422125 | 0.015243629 | 1.279259251 | 0.000439611 |
| PTEN | 0.914013957 | 0.504556192 | 0.039399212 | 0.143910783 | 1.811520644 | 0.000641675 |
| PKCa | 0.456910425 | 0.685572435 | 0.047698757 | 0.138207103 | 0.666465573 | 0.000699211 |
| c.JUN_pS73 | 1.006136589 | 0.708828011 | 0.115870307 | 0.121858154 | 1.419436835 | 0.00070545 |
| a.Tubulin | 1.003120187 | 0.774513722 | 0.082059239 | 0.098128633 | 1.295161286 | 0.000884047 |
| AMPKa | 0.436859037 | 0.607033367 | 0.03904812 | 0.105815391 | 0.719662313 | 0.000938561 |
| PTEN.138G50 | 1.240263585 | 0.603426629 | 0.365937621 | 0.186326156 | 2.05536767 | 0.000945558 |
| NBS1 | 0.14211307 | 0.399044618 | 0.025371057 | 0.21905263 | 0.356133284 | 0.001236694 |
| AIB1 | 0.471798082 | 0.645676264 | 0.039446088 | 0.110710201 | 0.730703773 | 0.001254419 |
| Cyclin.E1 | 0.241988287 | 0.453231317 | 0.026545469 | 0.160356461 | 0.533917842 | 0.001338894 |
| BAD_pS112 | 0.619547486 | 0.427306573 | 0.044253938 | 0.108484118 | 1.449889903 | 0.001935755 |
| S6_pS235 | 5.385752187 | 3.633016199 | 0.759775259 | 0.873583716 | 1.482446511 | 0.001979049 |
| HSP27 | 0.088889887 | 0.077851933 | 0.004911926 | 0.005578592 | 1.141781374 | 0.001993912 |
| XRCC1 | 0.250894253 | 0.322061401 | 0.010853126 | 0.048390367 | 0.77902615 | 0.002241178 |
| Cyclin.B1 | 4.327278784 | 9.399528662 | 2.17030685 | 3.335661529 | 0.460371891 | 0.002259383 |
| AKT | 1.48069539 | 0.975833441 | 0.132717774 | 0.256655662 | 1.517364878 | 0.002954693 |
| YBI | 0.646603654 | 0.507543953 | 0.085191009 | 0.05806186 | 1.273985533 | 0.003084537 |
| Cyclin.B1 | 2.174627532 | 4.268345819 | 0.90522439 | 1.404006796 | 0.509477822 | 0.003313811 |
| MAPK_pT202 | 2.267392447 | 1.144062982 | 0.647965498 | 0.42888824 | 1.981877295 | 0.003461053 |
| PKCa | 0.550054804 | 0.339295766 | 0.034736791 | 0.113008781 | 1.62116613 | 0.003931032 |
| ZNF342 | 0.316408205 | 0.420202025 | 0.009314226 | 0.0823791 | 0.752990673 | 0.004227772 |
| Cyclin.E | 0.086468844 | 0.098218713 | 0.003674399 | 0.008303872 | 0.880370365 | 0.004865277 |
| JUNB | 0.167895621 | 0.258467882 | 0.014995054 | 0.074227464 | 0.649580209 | 0.005422927 |
| Stathmin | 0.1229442 | 0.147197597 | 0.004532115 | 0.018962469 | 0.835232386 | 0.007116514 |
| SMAD3_pS423 | 0.949999223 | 0.673110996 | 0.078013936 | 0.176667033 | 1.411355969 | 0.007574451 |
| BIM.V | 0.062752516 | 0.096715957 | 0.005305906 | 0.032303803 | 0.648833125 | 0.007933903 |
| SMAD3 | 0.819219072 | 0.636648167 | 0.07248105 | 0.123758391 | 1.286768915 | 0.008315075 |
| p90RSK_pT359 | 0.155382049 | 0.226028573 | 0.003749312 | 0.065970472 | 0.687444276 | 0.009226147 |
| SRC_pY527 | 0.300030651 | 0.330055576 | 0.020242786 | 0.018501452 | 0.909030699 | 0.010181105 |
| Cyclin.E2 | 0.080850327 | 0.089866904 | 0.005294214 | 0.006183488 | 0.899667438 | 0.011162766 |
| HSP70 | 0.141095761 | 0.176949357 | 0.002268128 | 0.031325412 | 0.797379334 | 0.012885038 |
| PKCa_pS657 | 0.575457667 | 0.393686762 | 0.073947078 | 0.123826766 | 1.461714549 | 0.013235102 |
| LCK | 0.130625496 | 0.146612436 | 0.007286725 | 0.012797804 | 0.890957816 | 0.013929921 |
| COX2 | 1.817838712 | 0.983441812 | 0.755043842 | 0.453139861 | 1.848445622 | 0.01395324 |
| ERa_pS118 | 0.17254063 | 0.207110071 | 0.010326974 | 0.030805741 | 0.833086625 | 0.014305935 |
| AR.N20 | 0.265542006 | 0.354883603 | 0.013251788 | 0.08913207 | 0.748250986 | 0.014628095 |
| MAPK_pT202 | 1.113732161 | 0.659697674 | 0.272557066 | 0.229311046 | 1.688246307 | 0.014649845 |
| p90RSK_pT359 | 0.144041053 | 0.205274251 | 0.005434398 | 0.06171065 | 0.701700539 | 0.014778159 |
| AR.C19 | 0.261828155 | 0.20758835 | 0.020519497 | 0.043021325 | 1.261285399 | 0.015131243 |
| SMAC_DIABO | 0.536000728 | 0.753380508 | 0.045323626 | 0.206411607 | 0.711460839 | 0.015792213 |
| CD20 | 0.290865288 | 0.267930907 | 0.012844354 | 0.016915557 | 1.085598118 | 0.01672375 |
| Fibronectin | 0.42410535 | 0.205118855 | 0.13203637 | 0.193872486 | 2.06760783 | 0.016964377 |
| AR.N20 | 0.130991596 | 0.179906841 | 0.00602801 | 0.051440004 | 0.728107919 | 0.019204424 |
| ATRIP | 0.229798441 | 0.279415155 | 0.02181929 | 0.041430395 | 0.822426547 | 0.02035317 |
| Caveolin.1 | 4.755652577 | 2.199403803 | 1.629013819 | 2.991228888 | 2.162246228 | 0.020541529 |
| c.Myc_pT58 | 0.422502872 | 0.528767835 | 0.058668516 | 0.096156645 | 0.799032852 | 0.021065911 |
| MKLP.1.D17 | 0.230309584 | 0.26303369 | 0.014707742 | 0.028696833 | 0.875589681 | 0.021157188 |
| Erg.1_2_3 | 0.410442075 | 0.363985398 | 0.023236535 | 0.039687373 | 1.127633353 | 0.021267619 |
| CHK2 | 0.401026879 | 0.825096433 | 0.058956734 | 0.525737514 | 0.486036375 | 0.022898757 |
| CASK | 0.492300503 | 0.751055319 | 0.151882286 | 0.288799482 | 0.655478352 | 0.023569958 |
| p53 | 0.035826397 | 0.124294469 | 0.004226237 | 0.095992678 | 0.288238064 | 0.025379046 |
| Cofilin_pS3 | 0.346237209 | 0.228437482 | 0.092672779 | 0.0878754 | 1.515676001 | 0.025485898 |
| BOP1.N16 | 0.429226579 | 0.385085051 | 0.039897647 | 0.028196326 | 1.114627997 | 0.025561801 |
| PLK1 | 0.222964733 | 0.297190452 | 0.035843483 | 0.068121982 | 0.750241911 | 0.025902717 |
| TSC2_pT1462 | 0.893560544 | 0.693325906 | 0.108578551 | 0.16397596 | 1.288803052 | 0.027675477 |
| p21 | 1.22987335 | 0.844439492 | 0.219840638 | 0.419570685 | 1.456437509 | 0.027932641 |
| FOXO3a | 0.548814434 | 0.460243415 | 0.042833509 | 0.081500433 | 1.192443858 | 0.028041731 |
| Caspase.7.cleaved | 0.089003248 | 0.103285318 | 0.003637304 | 0.01460329 | 0.861722166 | 0.028792986 |
| FAK_pY397 | 0.735472219 | 0.561156246 | 0.094458416 | 0.174797551 | 1.310637143 | 0.029945909 |
| Cofilin | 0.442926477 | 0.376200481 | 0.039191505 | 0.063279258 | 1.177368183 | 0.038066178 |
| BRCA2 | 0.105413968 | 0.100419914 | 0.00314486 | 0.004583585 | 1.049731708 | 0.038111517 |
| eIF4E | 1.528373754 | 1.218549691 | 0.329844715 | 0.26734958 | 1.254256404 | 0.041742913 |
| Telomerase | 0.152065508 | 0.168360704 | 0.012598225 | 0.01498783 | 0.903212591 | 0.046021829 |
| BCl.XL | 0.172627638 | 0.299736435 | 0.015252275 | 0.201466269 | 0.575931447 | 0.048741592 |
| CHK2_pT68 | 0.136609011 | 0.203246592 | 0.023132263 | 0.083309829 | 0.672134323 | 0.0499792 |

Here we present a method for propagating a diverse array of ovarian carcinoma cell types. Use of this method, in the form of a novel medium, has yielded to date a panel of 25 new ovarian cancer cell lines that are extensively characterized, with histopathological and molecular analysis that includes whole genome profiles of DNA and RNA data and protein arrays.

The molecular profile of OCI cell lines we describe here demonstrate a remarkable consistency and robustness across the DNA, mRNA and protein profiles, and recapitulate clinically relevant patient populations. For example, a subset of the OCI cell lines have a gene expression profile that resembles tumors from patients with worse outcomes and are more resistant to Taxol, a first line treatment for ovarian cancer. This result shows that the in vitro drug responses of these OCI lines may indeed correlate closely with in vivo patient responses to drug treatments. Such a correlation between in vitro cell line data and in vivo patient data is especially encouraging since it has been recognized that such correlations are rare with standard tumor cell lines, which tend to be more drug-sensitive than human tumors, leading to false-positive hits in cell culture based drug screens. The closer correlation between OCI lines and human ovarian tumors is perhaps not surprising, since we observed that cytological, morphological and molecular features of the OCI lines and their xenograft tumors resembled specific subtypes of human ovarian cancer, which has not been the case for most SOC lines.

A robust and efficient culture system yielding cancer cell populations that predict patient responses to various drugs will greatly improve development of new drugs for personalized treatment of cancer patients. Our results suggest that this methodology can be adopted to culture other tumor types such as leukemias, breast cancers, pancreatic cancers, gastrointestinal sarcomas, etc. The methodology described herein can be adapted for personalized oncology where the drug sensitivity profile of each patient's tumor can be assessed real-time in cultured tumor cells, and this information can be used to guide treatment decisions.

### Methods

Primary tumor culture and cell lines: Fresh tumor tissue fragments were minced and plated on Primaria (BD Biosciences) plates before and after digestion with 1 mg/ml collagenase (Roche). The tumor cells were cultured in WIT nutrient medium described previously (Ince et al., Cancer Cell 12, 160-170, 2007), supplemented with insulin, hydrocortisone, EGF, cholera toxin, and serum. We refer to this version of the medium as WIT-OC. This formulation was supplemented with 17β-estradiol for endometrioid and mucinous tumors. The papillary serous, clear cell, dysgerminoma, and carcinosarcoma tumors were cultured in 5% CO₂ and regular O₂ at 37° C. as monolayers attached to Primaria culture plates. The endometrioid and mucinous tumors were cultured in 5% CO₂ and low O₂ at 37° C. as monolayers attached to Primaria culture plates. The tumor cells were passaged at a ˜1:3 ratio once a week and plated into a new flask at approximately 1×10⁴ cells/cm². During the initial weeks of culture, (˜1-5) the plates were treated with diluted trypsin first, in order to deplete stromal cells. The remaining cells that are still attached to the culture plate were treated with 0.25% trypsin for sub-culturing. In general, tumor cultures were free of stromal and normal cell types within 4-6 passages (see Supplemental Methods for further details of culture methods). The SOC cell lines were grown as per the instructions of the vendor. OCI lines will be available from the Ince laboratory upon publication. All study procedures were approved by the Institutional Review Boards at the Brigham and Women's Hospital (BWH) and Massachusetts General Hospital (MGH) to collect discarded tissues.

Protein, DNA and RNA analysis: Protein expression was analyzed by RPPA, as described previously (Hu et al., Bioinformatics 23, 1986-1994, 2007). Replicate data were averaged, log₂ transformed, median centered and subjected to hierarchical clustering using un-centered Pearson correlation in Cluster (v. 3.0) and Java TreeView (v. 1.1.1). mRNA expression for the cell lines was measured using the Illumina HumanHT-12 v4 Expression BeadChip platform. The gene expression data for 285 ovarian tumor samples were obtained from the Gene Expression Omnibus (GEO) (accession number: GSE9899) and normalized by RMA method. The genomic DNA from tumors and cell lines were analyzed with Affymetrix 250K Sty chips. The copy number analysis was performed using the Molecular Inversion Probe (MIP) 330k microarrays from Affymetrix.

Drug sensitivity experiments: The relative sensitivities of OCI and SOC cell lines to chemotherapy drugs was measured by seeding an equal number of cells in six replicates in 96-well black-walled clear bottom Corning plates at 3000 cells/well, and allowing attachment in WIT-OC for 12 h. Both OCI and SOC cell lines were exposed to drugs in WIT-OC medium. The cell lines were cultured in the presence of drug or vehicle control for 96 h. The fraction of metabolically active cells after drug treatment was measured by incubation with 2:10 (v/v) CellTiter-Blue reagent (Promega Cat#G8081) in media for 2 h, and the reaction was stopped by addition of 3% SDS. Fluorescence was measured in a SpectraMax M5 plate reader (Molecular Devices, CA) using SoftMax software (555EX/585EM).

Analysis of tumorigenicity: Single-cell suspensions were prepared in a Matrigel: WIT mixture (1:1) and 1-5 million cells per 100 μl volume were injected in one intraperitoneal and two subcutaneous sites per mouse. Tumor cell injections were performed on 6-8 week old female immunodeficient nude (Nu/Nu) mice (Charles River Laboratories International, Inc, Wilmington, Mass.). Tumors were harvested 5 to 9 weeks after implantation. Tumor histopathology was assessed with hematoxylin and eosin stained sections of formalin-fixed paraffin-embedded (FFPE) tissues. All mouse studies adhered to protocols reviewed and approved by either the BWH or MGH Institutional Animal Care and Use Committee.

### Supplemental Methods

Cell Culture Medium: Several different media have been previously used to culture human breast and ovarian cells including RPMI, DMEM, Ham's F12, MCDB-105, McCoy's 5A, and MCDB-170 (MEGM). In general only a small percent of primary ovarian or breast cancer samples can be established as cell lines using these standard cell culture media. Consistent with this, we failed to establish any permanent human ovarian cancer cell lines using these standard media to culture cells from more than one hundred tumors. Therefore, we explored the use of a chemically-defined serum-free cell culture media (WIT) that we had previously developed to support growth of human breast epithelial cells derived from the normal tissue as described in Ince et al., Cancer Cell 12, 160-170, 2007.

WIT media include a family of novel chemically-defined cell culture media that can support long-term growth of normal and transformed human breast cells without undefined components such as serum, feeder-layers, tissue extracts or pharmacological reagents (Ince et al., Cancer Cell 12, 160-170, 2007). Using a version of this medium optimized for normal cells (WIT-P), we were able to culture human breast epithelial cells (BPEC) for more than seventy population doublings during six months of continuous culture, a nearly 1021-fold expansion of cell number (Ince et al., Cancer Cell 12, 160-170, 2007). In contrast, in standard medium these normal breast epithelial cells ceased growing after several passages (Ince et al., Cancer Cell 12, 160-170, 2007). We initially tested a version of the medium (WIT-T) optimized for transformed human breast cells (BPLER) (Ince et al., Cancer Cell 12, 160-170, 2007) to establish human ovarian tumor cell lines, but were unsuccessful with more than a dozen tumor samples using this medium.

Next, we examined whether modifications in the concentration of distinct components of WI-T medium would support the growth of primary ovarian tumors cells. First, we reasoned that low levels of serum may be required to mimic the physiologic environment of normal ovary, fallopian tube and ovarian cancers in the peritoneal cavity. Normal human breast cells, like most epithelium, never contact blood or serum directly under physiologic conditions and thus the WIT medium we developed for normal breast epithelial cells was completely devoid of serum. In contrast, the ovaries and fallopian tubes are directly in contact with normal peritoneal fluid, which contains a physiologic serum protein concentration that can be as high as fifty percent of the circulating blood. Importantly, concentrations of proteins and growth factors in human ascites fluid present in ovarian cancer patients can be higher than serum. The addition of serum to WIT medium proved to be necessary, but not sufficient for growth of ovarian tumor cells; additional factors had to be optimized.

One of the difficulties associated with optimizing media is the non-obvious synergistic combinatorial effects of individual components. In many cases, individual additives did not increase ovarian cancer culture success incrementally; but cell growth increased exponentially when all components were added at optimal concentrations. After many years of optimization, we found that inclusion of insulin, hydrocortisone, EGF, and cholera toxin in addition to fetal bovine serum to WIT-T media showed broad efficacy in supporting the growth of the different ovarian tumor subtypes.

Some ovarian tumor subtypes, such as endometrioid and mucinous cancers, express estrogen receptor (ER), and addition of β-Estradiol (E2) enhanced the growth of these tumors subtypes.

We also had to optimize O₂ levels because while papillary serous and clear cell tumors proliferated best in ambient O₂ (18 to 21%), ER+ endometrioid and mucinous tumors proliferated best at low O₂ levels (5 to 10%), lower O₂ levels (1%) were detrimental. Furthermore, culture at lower O₂ levels (5%) was necessary to maintain ER expression. It has been shown that estrogens can play a role as pro-oxidants or anti-oxidants depending on the cell types. This might be one reason ER+ endometrioid and mucinous OCI lines in 100 nM β-Estradiol maintain their phenotype best in low O₂ levels.

A very time consuming aspect of this process was the need to validate the ability of each medium to support the derivation of multiple primary ovarian cancer samples to ensure that the final formula has applicability across the broad spectrum of specimens and cancer subtypes. While some ingredients had little effect on culture efficiency for some samples, they were absolutely essential for others. Importantly, the effects of removing these components became more apparent after multiple passages. Hence, effects of each ingredient had to be tested over many passages.

The cell attachment surfaces was also important; while uniformly negatively charged regular tissue culture plastic produced variable results, a modified cell culture plastic with mixed positive and negative charges (Primaria, BD) helped in preserving cell morphology and heterogeneity.

All of these factors—the non-obvious nature of combinatorial outcomes, the need to test conditions in multiple passages and in multiple cell lines, and the very large number of conditions to test—precluded an incremental approach to medium development. Furthermore, even leaving aside 30 to 50 differences between WIT and standard media, and just concentrating on the seven differences between WIT-T vs. WIT-OCe, at three concentrations would require examining 262,144 combinations. Therefore, it is not possible to systematically test each of these variations even in retrospect.

Despite these daunting odds, our empiric efforts led to identification of a combination of insulin, hydrocortisone, EGF, cholera toxin, serum, β-Estradiol, O₂ and cell culture flasks that supported long term culture of a majority of primary ovarian cancers. The medium optimized for human ovarian tumors, named WIT-OC (—OCe when β-Estradiol added), contains final concentrations of EGF (0.01 ug/mL, E9644, Sigma-Aldrich, St. Louis, Mo.), Insulin (20 ug/mL, 10516, Sigma-Aldrich), Hydrocortisone (0.5 ug/mL, H0888, Sigma-Aldrich), 25 ng/mL Cholera Toxin (227035, Calbiochem, EMD Millipore, Billerica, Mass.) together with 2-5% heat inactivated fetal bovine serum (HyClone, Thermo Fisher Scientific, Waltham, Mass.). With the current formulation of WIT-OC we were able to culture ovarian tumors with relatively high efficiency (25/26).

Lastly, we observed that none of the OCI lines we tested could be cultured in existing standard media. In contrast, all of the SOC lines we tested could be cultured in WIT-OC medium. To our knowledge, none of the standard media support the culture of all of the existing SOC lines, thus, it has been difficult to compare a large panel of SOC lines with each other. Our results indicate that WIT-OC medium has the potential to serve as a universal culture medium for SOC lines facilitating comparisons across cell lines.

Tumor Tissue Collection and Clinical Information: All study procedures were approved by the Internal Review Board at the Brigham and Women's Hospital to collect discarded tissues. In this initial study we concentrated on developing methods for successful culture of human ovarian tumors. For this purpose we used anonymized discarded human tissue and did not have access to clinical patient follow up information retrospectively. A prospective study with larger number of patients and clinical follow up will be needed to examine the direct comparison of individual patients to treatment and in vitro response of their corresponding cell line, which is underway.

Establishment of Cell Lines: Tumors are complex tissues composed of many cell types including stromal cells such as fibroblasts, endothelial cells, leukocytes, macrophages as well as normal epithelial cells that are intermingled with tumor cells. Among these, fibroblasts have historically been the easiest cells to grow in standard culture medium. In general serum promotes fibroblast growth and inhibits epithelial cell proliferation. When tumor tissue is cultured in medium with high serum content, typically there is an exponential growth of fibroblasts such that in a few weeks the fibroblasts completely overtake the culture plate, and soon all other cell types including tumor cells are eliminated. For this reason we used low levels of serum (2%) to culture ovarian tumor cells during the initial passages (1-5) to suppress fibroblast growth. Another difference between epithelial cell and fibroblasts is adherence to tissue culture plastic; in general epithelial cells are more strongly adherent to the culture flasks and require higher concentrations of trypsin to release them. Thus, it is possible to treat the plates with diluted trypsin first (0.05%), which selectively removes stromal cells. Afterwards, the epithelial cells that are still attached to the culture plate can be were treated with 0.25% trypsin for sub-culturing. WIT-OC was designed to support epithelial tumor cell proliferation and suppress fibroblast growth. However, in general it takes 4-6 passages with differential trypsinization to establish tumor cultures free of stromal and normal cell types. Afterwards the FBS levels were increased to 5% to increase tumor cell proliferation.

All OCI cell lines we cultured for at least 20-25 population doublings. In several cases, we carried out a formal population doubling analysis, which showed that the OCI lines can proliferate for at least 120 days (˜60 population doublings). Even though the mRNA extracts (FIG. 3) and the protein extracts (FIG. 4) were prepared at different times (passages) by different investigators and he drug sensitivity experiments (FIG. 1) were carried out separately by different authors, we observed a remarkable degree for consistency between mRNA, Protein profiles and drug response. These results indicate that these cell lines have a robust and stable phenotype.

Clonal Selection: Mindful of the possibility of clonal selection, we carefully monitored all OCI cultures for emergence of fast growing colonies, eliminated plates with too few starting cells and avoided partial trypsinization of plates during sub-culturing of OCI lines.

Measures of cell proliferation: In many previous reports, the cumulative number of cell passages has been used to indicate successful establishment of cell lines. However, it is important to note that the number of passages is not adequate by itself to verify net increase in tumor cell numbers. The cell passage number refers to the number of times the cells are successfully lifted from one plate and seeded into a new culture plate. This indicates that at least some of the cells can tolerate the transfer and are still alive. However, passage number does not necessarily correlate with increased cell numbers. For example, we were able to passage the tumor cell line OCI-05x in MCDB-105/M199 for nearly 20 passages. However, the population doubling curve of these cells stayed flat after 7 passages. Thus, there was no net increase between passages 7 and 20. Hence these cells, when grown using MCDB-105/199, could not provide a practical platform to carry out many experiments. The utility as a platform is better assessed by measuring population doublings.

An objective comparison of results from different studies can be made with previous work in terms of ‘population doublings’, or the log 2 of the number of cells harvested less the log 2 of the number of cells seeded; hence 2 cells expand to 1,024 cells in 10 population doublings (2¹⁰=1,024). Each 10 population doublings is approximately equal to 3 orders of magnitude (×10³) net increase in cell numbers, and so 20 population doublings would be close to a 1 million-fold increase and 30 population doublings would be close to a 1 billion fold increase in net cell numbers. We have achieved 30-100 population doublings with OCI lines, with no decrease in cell growth rate. At this point we ended long-term cell growth experiments, thus the upper limit of population doublings that can be achieved is likely to be much greater with OCI lines. Sixty population doublings would be approximately equal to 10²⁰-fold expansion in cell numbers (˜100 quintillion cells) more than adequate for any research use of these cell lines.

The growth rate plateau that is seen during the culture of tumor cells in standard media is linked to the long lag time between the initial plating of tumor tissue and the emergence of a cell line. This is a significant variable in evaluating the efficiency and practicality of a culture system, and has significant implications for the quality of the cell lines. For example, it was reported that on average it took more than five months (21 weeks) before tumor cells could be passaged for the first time, which is similar to our experience using RPMI medium (Verschraegen et al., Clinical cancer research: an official journal of the American Association for Cancer Research 9, 845-852, 2003).

In standard cell culture medium both normal and ovarian tumor cells are growth arrested within the initial several passages. Since the growth arrest due to telomere-shortening occurs typically after 50-70 passages, this type of early growth arrest is due to inadequate culturing conditions.

Soft agar colony assay: In order to confirm that the OCI cell lines we established maintained their transformed phenotype in culture we carried out anchorage independent growth assays in soft agar. Since normal cells are in capable of forming soft agar colonies, this is an excellent method to ensure that we have indeed established tumor cell lines. For these assays, well bottoms of a 12-well plate were sealed with 0.6% agar prepared in WIT-OC medium to prevent monolayer formation. Cells from established cultures (passage 6-8) were harvested. A single cell suspension in 0.4% agar in WIT-OC medium was added and allowed to set at room temperature, and placed in 37° C. incubators with 5% CO₂. The cells were fed with 0.4% agar in WIT-OC at 2 weeks, and colony formation was assessed 2-4 weeks after plating.

Alternatively, tumor cells were grown in suspension cultures. The tumor spheres were grown in WIT-OC medium with 2% B27 supplement (Gibco), 20 ng/ml EGF, 20 ng/ml bFGF (BD Biosciences), 4 ug/ml heparin, and 0.5% methyl cellulose. For sphere formation experiments, 15,000-20,000 cells/well were plated into 6-well ultra-low attachment plates (Corning), fed at days 1, 3,5, and spheres were counted at day 7.

LOH Analysis: The genomic DNA of tumor tissues were extracted from paraffin sections or, when available, from fresh tissues. The fresh tumor tissues were homogenized directly in RLT+ cell lysis buffer (Qiagen). The DNA was extracted from the lysates using the Qiagen All-Prep mini kit. Briefly, DNA is cleaved with Sty1, and the fragments are PCR amplified. The purified products were further fragmented with DNaseI, biotinylated, hybridized to a chip, and fluorescently labeled with phycoerythrin-conjugated streptavidin with signal amplification. Inferred LOH analysis was performed using dCHIP software and employed the hidden Markov model with a reference heterozygosity rate of 0.2.

LOH segment analyses was performed using the Affymetrix Genotyping Console (version 4.1.4.840). The BRLMM algorithm was used for genotyping (score threshold=0.5, prior size=10,000, DM threshold=0.17). Unpaired sample analysis was performed for CN and LOH using 20 female samples taken from the HapMap samples that Affymetrix has provided for the platform (default configuration, i.e. quantile normalization, 0.1 Mb genomic smoothing). Then the Segment Reporting Tool within the software was run to get the filtered result (minimum number of markers per segment=5, minimum genomic size of a segment=100 kbp). Finally, after synchronizing the probe sets for all the samples, we further summarized the LOH calls for every 60 kbp region along the chromosomes. When there were no LOH calls in such a region for all samples, the region was excluded from the final table.

Copy Number Analysis: The copy number analysis was performed using the Molecular Inversion Probe (MIP) 330k microarrays from Affymetrix. MIP probes are oligonucleotides in which the two end sequences are complementary to two adjacent genomic sequences; these two ends anneal to the genomic DNA in an inverted fashion with a single base between them. In copy number analysis the genomic DNA is hybridized to the MIP probe and the reaction split into two separate tubes containing nucleotide mixes (triphosphates of either Adenine+Thymine or Cytosine+Guanine). With the addition of polymerase and ligase, the MIP probe circulates in the presence of the nucleotide complementary to the allele on the genome. Genomic DNA is limiting in the reaction such that the number of circulated probes proportionally reflects the absolute amount of template DNA. After circularization, unused probes and genomic DNA are eliminated from the reaction by exonuclease leaving only circularized probes. These probes are then amplified, labeled, detected, and quantified by hybridization to tag microarrays; tags are designed to have low cross hybridization. The data was analyzed using the Nexus 5.1 software from BioDiscovery.

In order to compare CNV patterns of OCI cells with the ovarian tumors in the TCGA dataset, we downloaded the MSKCC Agilent 1M Copy Number Variation data from the TCGA data portal. This set included 497 copy-number segmentation files generated from 487 TCGA ovarian samples using the CBS algorithm. We randomly selected 100 files and merged individual copy number profile into a single consensus using an interval-merging algorithm that sums together the mean log 2 intensity values in overlapping intervals.

Similarly, we generated individual copy number profile for 25 ovarian cell-line samples using Affymetrix MIP array and the CBS algorithm in the DNAcopy package in R bioconductor. A consensus copy number profiling was generated from these 25 samples using the same interval-merging algorithm. Method for Copy Number/LOH.

RNA expression analysis: Total RNA was extracted from each cell line in triplicate (different passages from the same cell line) using the RNeasy Mini kit (Qiagen, Valencia, Calif.) according to the manufacturer's instructions. RNA was checked with a size fractionation procedure using a capillary electrophoresis instrument (Bioanalyzer 2100, Agilent Technologies, Santa Clara, Calif.) to ensure high quality and RNA concentrations were estimated using the Nanodrop ND-1000 (Nanodrop Technologies Inc, Wilmington, Del.). Gene expression for the cell lines was measured using the Illumina HumanHT-12 v4 Expression BeadChip platform. Raw signals of all the built-in controls were checked as quality control for the performance of the arrays. The sample-independent controls were used to check hybridization and signal generation and the housekeeping genes were used as sample-dependent controls. After background subtraction, the data were normalized across arrays using quantile normalization (Bolstad et al., 2003). The average signal intensities were used for gene expression profiling.

Gene expression data for 285 ovarian tumor samples were also obtained from the Gene Expression Omnibus (GEO) (accession number: GSE9899). The samples were assayed using the Affymetrix HG-U133 Plus 2.0 platform. The data were normalized by RMA method (Irizarry et al., Nucleic acids research 31, e15, 2003). The two data sets were combined by matching gene symbols. The data were median-centered for each sample. Genes with an expression level that had at least a 2-fold difference relative to the median value across tissues in at least 4 cell lines were selected. This resulted in 3831 genes for further analysis.

In FIG. 3 the combined samples were clustered using hierarchical clustering to see whether the cell lines could be grouped with patient samples with different clinical outcomes. This was done using a Spearman correlation coefficient based distance matrix and Ward's minimum variance based agglomeration algorithm. The sample tree was cut into three main branches (FIG. 1). Cluster P2 has all the SOC cell lines (and a few OCI cell lines), and all the OCI cell lines were grouped in Cluster P1. Branch 3 was not included in survival analysis because it is small and has no cell line samples. The Kaplan-Meier curves for progression free survival and overall survival were plotted in FIG. 1. All the statistical analysis, after the raw data had been generated from the platform vendor software, was performed in R (Team, 2011).

Comparison of the mRNA expression of cell lines with primary tumor tissue is challenging, because primary tumors are a heterogeneous mix of normal cells, tumor cells, stromal cell, inflammatory cells, apoptotic cells, blood vessels, necrotic matrix etc. Furthermore, cell lines in culture have a much higher cell in cycle in exponential growth phase compared to tumors. Hence, comparisons of tissues with cell lines generally result in cell cycle, stroma, matrix genes, inflammatory genes dominating the profile. Since we had limited fresh tumor material that was mostly used for optimizing culture conditions, we did not have enough tissue material to carry out microdissection that may address some of these problem. For these reasons we were not able to compare the primary tumor tissue with cell lines.

Methods used for microarray data based pathway enrichment analysis: For pathway analysis in FIG. 6 we used average value of expressions in log 2 transformed microarray data, for each gene on each sample, detected by different probes to denote the consensus gene expression. In MATLAB 2010b, student's t-test p-value and fold-change value were calculated for each gene with the partition of clusters 1 and 2. Gene names and their affiliated p values, fold-change values were imported into Ingenuity Pathway Analysis (IPA). By setting cutoffs as 0.05 and 1 for p and fold-change values (log 2-based, either up- or down-regulation), 823 genes were identified as significantly differentiate expressed. 558 and 265 genes were found up-regulated in clusters 1 and 2 respectively. Using the ‘core analysis’ module in IPA, 37 and 41 pathways were found significantly enriched (with the p value <0.05 by IPA) correspondingly for cluster 1 and 2 as up-regulated.

Protein expression analysis: In FIG. 4 the cell lysates were immobilized on nitrocellulose coated slides, and each slide was incubated with an antibody specific for a protein of interest. The protein lysates were prepared in a lysis buffer containing SDS and protease inhibitors. Semi-confluent wells in 6-well plates were lysed in 125 uL lysis buffer on ice in triplicate (at least two different passages from the same cell line). Sample concentrations were adjusted after BCA measurements. Each sample was spotted onto the slide in dilution series (5 dilutions), and the slides were probed with 156 (first experiment) and 191 (second experiment) primary antibodies and the signal intensity was captured by a biotin conjugated secondary antibody and amplified by a DakoCytomation-catalyzed system. The slides were scanned, analyzed and quantitated using MicroVigene software (Vigene Tech inc. Carlise, Mass.) to generate spot signal intensities, which were processed by the R package SuperCurve. Protein concentrations were derived from the supercurve for each lysate by curve-fitting and normalized by median polish. The antibodies utilized in this study were primarily targeting proteins involved in PI3K/Akt pathway or were otherwise cancer related signaling pathways. The signal intensity data was collected and normalized using software specifically developed for RPPA analyses. Replicate data were averaged, log 2-median centered, hierarchically clustered (Cluster 3.0), and visualized in heatmaps (Java TreeView 1.1.1). Two-sided Student's tests of log transformed RPPA values were performed using the t.test function in bioConductor/R.

Cell Line Unique Identifier mtDNA: A common problem in cell culture is cross-contamination or misidentification of cells. In repeated studies since 1970s, it has been shown that 15-25% of cell lines are contaminated with a second line, or is completely misidentified. In the 1970s and 1980s, it was shown that over 100 cancer cell lines were actually HeLa cells. An effective cell culture quality and identity control is required in order to avoid inter- and intra-species contamination of cell lines and their further propagation and dissemination. However, vigilant monitoring against misidentification and cross-contamination is possible by developing a practical “unique identifier” for the cells by the establishing laboratory.

We generated mtDNA sequence evidence that 16 cell lines examined in this manuscript are from unrelated individuals. Thus, the OCI cell lines can be verified by the recipient laboratories and can be monitored for purity and integrity. This will significantly reduce the incidence of cell line contamination and misidentification. The control region of the human mtDNA is highly polymorphic due to a rapid rate of evolution. The mtDNA does not undergo recombination and is present in high copy number per cell. For this reason, its analysis is very useful for the identification of cell lines.

DNA was extracted using the QTAamp DNA Mini Kit using standard methods. The HVI and HVII segments were amplified by PCR using specific primers. The two segments were directly sequenced by capillary electrophoresis on both strands. Nucleotide substitutions and insertions/deletions were found by comparison with Cambridge reference sequence (NCBI Reference Sequence NC_012920.1). PCR amplification was performed in 50 μl with a Bio-Rad thermocycler (Applied Biosystems Inc., USA). The PCR product amplified from D-loop mtDNA was detected by electrophoresis on a 1% agarose gel with 1×TBE buffer at 120 V and 60 mA for 60 min and under UV transillumination after ethidium bromide staining, and photographed. After purification with the QIAquick Gel Extraction Kit (QIAGEN, USA), all of the PCR products were sequenced (Operon, Petaluma, CA) in both directions using the same primers as PCR. After nucleotide sequencing, sequence variations were determined by comparison with the Cambridge reference sequence using CLUSTALW2.

Drug sensitivity experiments: The relative sensitivities of OCI and SOC cell lines to Taxol was measured by seeding 3000 cells/well in six replicates in 96-well black-walled clear bottom Corning plates and allowing attachment in WIT-OC for 12 h. Both OCI and SOC cell lines were cultured in the presence of Taxol dosages ranging from 1 to 800 nM (or vehicle control) in WIT-OC medium for 120 h. The fraction of metabolically active cells after drug treatment was measured by incubation with 2:10 (v/v) CellTiter-Blue reagent (Promega Cat#G8081) in media for 2 h, and the reaction was stopped by addition of 3% SDS. Fluorescence was measured in SpectraMax M5 plate reader (Molecular Devices, CA) using SoftMax software (555EX/585EM). In case of high variation among calculated values for four independent assays, four additional independent assays were performed to allow defining and discarding outliers.

Lethal Dose Analysis: Data was analyzed using GraphPad Prism 5 Software, and values were fit to a dose response-inhibition curve with variable slope (sigmoidal with four parameters). The cell viability as response r between bottom (B) and top (T) values (B<r<T) was assumed to depend on concentration (C) via a general Hill equation for inhibition as in equation (1)

$$r = B + ( T - B ) \frac{{C}^{n}}{{C}^{n} + {IC}_{50}^{n}} \qquad ( 1 )$$

where IC₅₀ is the concentration producing a response that is halfway between Bottom and Top (notation as used in Prism) and n is the Hill coefficient. T was constrained to be constant and equal to 100 and B to be equal or greater than zero. Accordingly, the concentration that produces a given response r (viability) can be calculated from equation (2), and after obtaining B, T, IC₅₀, and n from fitting the data, this was used to estimate LD₉₀ values corresponding to concentrations causing 90% lethality (r=10% viable cells).

$${C}_{r} = {{IC}_{50} ( \frac{r - B}{T - r} )}^{1 / n} \qquad ( 2 )$$

### Example 3

### Gene Expression Signature of Normal Cell-of-Origin Predicts Ovarian Tumor Outcomes Tumors

Most epithelial ovarian cancers are thought to arise from different cells in the ovarian or fallopian tube epithelium. We hypothesized that these distinct cells-of-origin may play a role in determining ovarian tumor phenotype and also could inform the molecular classification of ovarian cancer. To test this hypothesis, we developed new methods to isolate and culture paired normal human ovarian (OV) and fallopian tube (FT) epithelial cells from multiple donors without cancer and identified a cell-of-origin gene expression signature that distinguished these cell types within the same patient. Application of the OV versus FT cell-of-origin gene signature to gene expression profiles of primary ovarian cancers permitted identification of distinct OV and FT-like subgroups among these cancers. Importantly, the normal FT-like tumor classification correlated with a significantly worse disease-free survival. This work describes a new experimental method for culture of normal human OV and FT epithelial cells from the same patient. These findings provide new evidence that cell-of-origin is an important source of ovarian tumor heterogeneity and the associated differences in tumor outcome.

Studies investigating the molecular basis of ovarian tumor heterogeneity have identified distinct transcriptional subtypes of ovarian cancer based on their gene expression signatures. Understanding the source of this molecular heterogeneity is crucial to highlight aberrant genes or pathways that could be targeted to improve treatment outcomes through subtype-stratified care. Our objective was to investigate the role of ovarian and fallopian tube cell-of-origin in determining the associated tumor behavior and to define their contribution to the molecular heterogeneity observed in ovarian cancer. Towards this goal, we developed a new cell culture medium and methods to culture and propagate normal ovarian epithelium and fallopian tube epithelium as paired cultured cells from the same individuals. We then identified a gene signature that distinguished normal ovarian epithelium and fallopian tube epithelium from the same patients and applied this information to classify primary ovarian tumors as fallopian tube (FT)-like and ovarian epithelial (OV)-like; this classification was predictive of patient outcome. These findings provide new evidence that cell-of-origin is an important source of ovarian tumor heterogeneity and the associated differences in tumor phenotype.

### Materials and Methods

Tissue collection and culture of normal human fallopian tube and ovarian epithelium. Scrapings from the normal ovary and fallopian tube were collected using a kittner (e.g., Aspen Surgical) from two postmenopausal donors who were being treated at the Brigham and Women's Hospital for benign gynecologic disease following an IRB approved protocol to collect discarded tissues (see Supplementary Methods below). The cells used in this study are primary cell cultures established directly from tissue samples during the course of this study by the investigators. Collected cells were immediately placed in WIT-fo cell culture media and transferred to a tissue culture flask with a modified surface (Primaria, BD, Bedford, Mass.) and incubated at 37° with 5% CO₂ in ambient air. WIT medium was previously described (Stemgent, Cambridge, Mass.) and WIT-fo is a modified version of this medium optimized for fallopian tube and ovarian epithelial cells (see Supplementary Methods below). After 10-15 days, during which the medium was changed every 2-3 days, cells were lifted using 0.05% trypsin at room temperature (˜15 seconds exposure), then trypsin was inactivated in 10% serum-containing medium, followed by centrifugation of cells in polypropylene tubes (500×g, 4 minutes) to remove excess trypsin and serum. Subcultures were established by seeding cells at a minimum density of 1×10⁴/cm² (a split ratio of 1:2 was generally applied, i.e. one flask of cells was split and seeded into two equivalent-sized flasks). However, we highly recommend counting cells to seed at the required minimum density rather than relying on a split ratio. Medium was replaced 24 hrs after re-plating cells and every 48-72 hours thereafter.

To culture ovarian epithelial cells, we tested several previously described cell culture media (14-16), including a 1:1 mixture of MCDB 105/Medium 199 with a range of 5-10% fetal bovine serum, 2 mM 1-glutamine with and without 10 ng/ml epidermal growth factor, and Dulbecco's modified Eagle's medium (DMEM)/Ham's F-12 (1:1 mixture) with 10-15% fetal bovine serum. In neither case were we able to propagate ovarian epithelial cells beyond a few population doublings. For fallopian tube epithelium culture we tested several previously described media (17-19), a 1:1 mixture of DMEM/Ham's F12, supplemented with 0.1% BSA, 5% serum (1:1 mix of 2.5% fetal bovine serum plus 2.5% Nu Serum) and 17β estradiol, or a slightly modified version of this medium supplemented with 2% serum substitute. None of the above-mentioned traditional cell culture media that we tested supported long-term propagation of normal epithelial cells from human ovary or fallopian tube. Cell immortalization and transformation of the normal cells with defined genetic elements, and the analysis of tumorigenicity was carried out as previously described (see Supplementary Methods below).

Western blotting, live cell imaging and FACS. Protein expression was determined by immunoblotting of total cell proteins on Bis-Tris gels (Invitrogen, Carlsbad, Calif.) that were transferred onto PVDF membranes and probed with antibodies for Cytokeratin 7 (MAB3554) (Millepore, Billerica, Mass.), PAX8 (10336-1-AP) (ProteinTech Group, Inc, Chicago, Ill.), FOXJ1 (HPA005714), HOXA5 (ab82645) (Abcam, Cambridge, Mass.), and HOXC6 (ab41587) (Abcam) and β-Actin (clone AC-15) (Sigma-Aldrich, St. Louis, Mo.) (see Supplementary Methods below). Cells were grown for two days on fluorodishes (World Precision Instruments, Sarasota, Fla.) and images of live cells were taken at 40× magnification using the Nikon TE2000-U inverted microscope. Fluorescence activated cell sorting (FACS) analysis using a FACS Aria multicolor high speed sorter (BD) was used to quantify ovarian and fallopian tube cells that were GFP positive following infection with pmig-GFP-hTERT.

Expression profiling and microarray analysis. Total RNA was extracted using the RNeasy Mini kit (Qiagen, Valencia, Calif.) and quality was verified using a Bioanalyzer (Agilent Technologies, Santa Clara, Calif.). Between 5-15 μg of RNA was used to generate biotinylated cDNA target that was hybridized to Affymetrix HG U133 Plus 2.0 microarrays (Affymetrix Inc., Santa Clara, Calif.) at the Dana-Farber Cancer Institute Microarray Core Facility. Microarray CEL files are available at GEO (GSE37648).

The OV/FT signatures were compared to publically available datasets that were generated with similar methods in order to minimize platform related and methodological bias. Hence, datasets generated from analyzing total unamplified RNA isolated from fresh frozen ovarian cancers and profiled using the same (HG U133 Plus 2.0) or a similar (HG U133A) Affymetrix microarray platform were used in these comparisons. We also prioritized datasets with the largest number of samples (TCGA) and those which contained non-serous tumors (Wu et al., (Cancer Cell 2007; 11: 321-33, Tothill et al., Clin Cancer Res 2008; 14: 5198-208). Affymetrix microarrays of four hTERT immortalized cell lines (OCE, FNE) from two patients as well as publically available ovarian cancer datasets by Wu et al. (Cancer Cell 2007; 11: 321-33) (GEO Series accession number GSE6008) and Tothill et al. (Clin Cancer Res 2008; 14: 5198-208) (GSE9891) were independently normalized using vsnrma (Huber et al., Bioinformatics 2002; 18 Suppl 1: S96-104). The TCGA mRNA expression data was normalized by the TCGA consortium.

We applied hierarchical clustering based on global expression profiles to the OCE/FNE cells and observed the strongest separation by patient (1 or 2) then by cell type (ovary or fallopian tube). To identify genes that were differentially expressed between paired hTERT immortalized human fallopian tube vs ovarian epithelium in the same patients, we applied a modified t-test (False Discovery Rate (FDR) adjusted P<0.05) using the duplicate correlation function in Limma to block for patient differences.

To classify human ovarian tumors as fallopian tube (FT)-like and ovary (OV)-like from three publically available gene expression datasets, we selected the most highly significant ten probesets with unique gene symbols that were over-expressed in either FNE or OCE and calculated the sum of the normalized expression values of these ten probesets in two ovarian cancer datasets by weighting FNE genes by (+1) and OCE genes by (−1) to calculate an overall signature expression score for each tumor (a higher score tumor is more FT-like). We then fit a bimodal distribution of Gaussian curves to this score using mixture modeling to classify ovarian tumors as OV-like or FT-like.

We compared the clinical characteristics of patient tumors classified as FT-like or OV-like using ordinal logistic regression (grade, stage) or Fisher's Exact Test (histologic subtype). Kaplan-Meier plots and univariate P-values using the log-rank test as well as multivariate Cox proportional hazards tests were calculated to evaluate the association of the FT/OV-like classification with survival. All analyses were conducted using R version 2.10.1.

### Results

Establishment of normal ovarian and fallopian tube epithelial cultures. The normal human ovarian epithelium and fallopian tube epithelium cells were collected from separate scrapings of the ovarian surface and the fimbriated end of the fallopian tube using an endoscopic kittner from two postmenopausal patients undergoing surgery for benign gynecologic conditions at the Brigham and Women's Hospital following an IRB-approved protocol to collect discarded tissues.

In order to culture normal human primary ovarian and fallopian tube epithelial cells, we modified the chemically-defined WIT medium that previously described. Next, we compared the long term growth of these cells in this modified medium optimized for fallopian tube and ovary cells (WIT-fo) with other media that have been previously used to culture ovarian epithelium and fallopian tube epithelium (Auersperg et al., Lab Invest 1994; 71: 510-8 and Comer et al., Hum Reprod 1998; 13: 3114-20) by plating cells from the same donor in replicate plates in either WIT-fo or control media conditions. It was possible to propagate both normal ovarian epithelium and fallopian tube epithelium in WIT-fo medium beyond 10 population doublings, which corresponds to >1000-fold net increase in cell numbers. In contrast, neither ovarian epithelium, nor fallopian tube epithelium could be propagated in traditional media beyond a few population doublings. We were not able to establish long-term cultures of normal ovarian epithelium or fallopian tube epithelium in any of the previously described media, including the unmodified WIT medium that was originally optimized to culture normal human breast cells (Ince et al., Cancer Cell 2007; 12:160-170) (see Supplementary Methods below).

To determine the origins of the cultured ovarian and fallopian tube epithelial cell populations, we investigated cell subtype specific markers in sections of normal human ovarian and fallopian tube formalin-fixed paraffin embedded (FFPE) tissues from 6 patients. Three antibodies (PAX8, FOXJ1 and CK7) distinguished ovarian surface from ovarian inclusion cyst epithelium, and ciliated epithelium from non-ciliated fallopian tube epithelium (see Supplementary Methods below). Both ovarian surface and inclusion cyst epithelia were CK7⁺. The ovarian surface epithelium was PAX8⁻ (mesothelial phenotype), except rare cells that were PAX8⁺; in contrast, the epithelium in >75% of the ovarian inclusion cysts was entirely composed of PAX8⁺ cells (Mullerian phenotype) (see Supplementary Methods below). In the fallopian tube, non-ciliated epithelium was CK7⁺/PAX8⁺/FOXJ1⁻, and the ciliated cells were CK7⁻/PAX8⁻/FOXJ1⁺ which is most consistent with the staining profiles of ovarian inclusion cyst epithelium and non-ciliated fallopian tube epithelium and these cultured cells are hereafter referred to as OC (ovarian epithelium) and FN (fallopian tube non-ciliated).

Immortalization of ovarian and fallopian tube epithelial cultures. Next, we introduced hTERT into the OC and FN cells to create immortalized derivatives (OCE and FNE cells, respectively). Immunoblotting showed that cultured ovarian and fallopian tube epithelium were CK7⁺/PAX8⁺/FOXJ1⁻ as expected and immunofluorescence confirmed that all of the OCE and FNE cells had a uniform PAX8⁺/FOXJ1⁻ phenotype. The OCE and FNE cells could be distinguished based on HOXA5 and HOXC6 expression. Western blotting confirmed that OCE cells are HOXA5⁺/HOXC6⁺ while in contrast these proteins were not detectable in FNE cells.

The immortalized OCE and FNE cells were cultured continuously beyond 40 population doublings, which corresponds to a ˜10¹²-fold net increase in cell numbers. In contrast, replicate plates of the same cells cultured in standard media (see Supplementary Methods below) or when transferred to unmodified WIT medium ceased growing after a few passages.

Immortalization of normal human ovarian epithelial cultures has been previously attempted using viral oncogenes such as HPV E6/E7 and SV40T/t (Maines-Bandiera et al., Am J Obstet Gynecol 1992; 167: 729-35 and Tsao et al., Exp Cell Res 1995; 218: 499-507) however this method also increases genetic instability and can cause the accumulation of DNA mutations that could significantly alter the gene expression profiles in the immortalized cells as compared with their finite lifespan counterparts. Furthermore, these SV40T/t and E6/E7 transformed cells are not immortal because the genetic instability eventually results in crisis and cell death within weeks to several months of continuous culture. Thus, using the WIT-fo media we have developed the first practical and robust system that allows long-term culture of hTERT immortalized OCE and FNE cells.

Application of a cell-of-origin (ovary vs. fallopian tube) gene signature to classify patient ovarian cancers. Based on studies suggesting that some ‘ovarian’ cancers may arise in the fallopian tube and others in the ovarian epithelium, we reasoned that it would be of value to determine if FNE and OCE cells expressed different gene signatures and that this cell-of-origin signature could be tested for its potential utility to distinguish these distinct subgroups of human ovarian cancer. Gene expression profiles of FNE and OCE cells were examined using HG U133 Plus 2.0 arrays. Application of a modified t-test (FDR adjusted P<0.05) using Limma while blocking for patient differences identified 632 and 525 probesets that were significantly up-regulated in FNE or OCE cells, respectively.

From this list we selected the top ten most highly significant differentially expressed probesets with unique gene symbols. Five of these genes are over-expressed in cultured fallopian tube cells (FNE genes: DOK5, CD47, HS6ST3, DPP6, OSBPL3) and the other five genes are over-expressed in cultured ovarian cells (OCE genes: STC2, SFRP1, SLC35F3, SHMT2, TMEM164). In preliminary analyses we determined that including additional probes with less significant differential expression was counterproductive; the inclusion of 20 or 100 highly significant probesets only appeared to introduce noise into the classification. Hence, all further analyses were carried out with the ten probesets.

Nucleic acid sequences for these genes are well known in the art and can be found in the National Center for Biotechnology Information (NCBI) database by their names and/or accession numbers. Examples of accession numbers (NCBI Reference Sequence numbers) for these genes are as follows: Homo sapiens docking protein 5 (DOK5): NM_018431; Homo sapiens CD47 molecule (CD47): NM_001777, NM_198793, BCO37306; Homo sapiens heparan sulfate 6-O-sulfotransferase 3 (HS6ST3): NM_153456, NM_205551, XM_926275, XM_931159, XM_941593, XM_945293; Homo sapiens dipeptidyl-peptidase 6 (DPP6): NM_130797, NM_001936.4, NM_001039350.2, NM_001290253.1, NM_001290252.1; Homo sapiens oxysterol-binding protein-like protein OSBPL3 (OSBPL3): AF392444, XM_005249698.1, NM_015550.3, NM_145320.2, NM_145321.2, NM_145322.2; Homo sapiens stanniocalcin 2 (STC2): NM_003714; Homo sapiens secreted frizzled-related protein 1 (SFRP1): NM_003012.4, BCO36503.1; Homo sapiens solute carrier family 35, member F3 (SLC35F3): NM_173508, XM_939358; Homo sapiens serine hydroxymethyltransferase 2 (mitochondrial) (SHMT2): NM_005412, NR_029416.1, NR_029415.1, NR_029417.1, NM_001166356.1, NM_001166357.1, NM_001166359.1, NM_001166358.1, NR_048562.1; and Homo sapiens transmembrane protein 164 (TMEM164): NM_017698, XM_001714477, XM_002343838, NM_032227. These sequences and accession numbers are incorporated herein by reference in their entirety.

In order to investigate the cellular origins of human ovarian cancers, we examined the expression profile of these ten probesets in previously published datasets. An overall signature expression score was calculated for each sample by weighting genes that are up-regulated in FNE by (+1) and genes that are up-regulated in OCE by (−1) and a bimodal distribution of Gaussian curves was applied to these scores together with mixture modeling to predict two subpopulations; those that were FT-like with high scores or OV-like with low scores.

We first validated the ten probeset FNE vs. OCE cell-of-origin signature in previously published datasets that had profiled normal human fallopian tube epithelium and normal ovarian surface epithelium. It is worth noting that in the previously published studies a single cell type was analyzed; the normal ovarian cells or the tubal cells were profiled in separate studies. Thus, different collection methods and analysis platforms were used, and ovarian and tubal cells were not patient matched. Despite these differences, the ten probeset FNE vs. OCE cell-of-origin signature correctly classified all of the microdissected FTE samples as fallopian tube (FT)-like (n=12) and all of the cultured OSE cells as ovary (OV)-like (n=6) in two different datasets. In addition, all four uncultured normal OSE scrapes in the Wu et al. (Cancer Cell 2007; 11: 321-33) dataset were correctly classified as OV-like.

The ten probeset gene signature was next used to classify 99 manually microdissected serous, endometrioid, clear cell and mucinous ovarian carcinomas in the Wu et al. (Cancer Cell 2007; 11: 321-33) dataset. Due to platform differences 8/10 probesets were available for analysis. The FNE vs. OCE signature expression scores visualized in a density plot showed a clear bimodal distribution which supports our binary classification of ovarian tumors into FT-like and OV-like subgroups. Comparisons of the clinical features of FT-like and OV-like tumors demonstrated that FT-like tumors were of significantly higher stage, higher grade and were predominantly composed of serous adenocarcinomas (P<0.001 for all comparisons) (FIG. 7a). In contrast, OV-like tumors included non-serous subtypes and lower grade cancers.

To further validate these results, we next evaluated the FNE vs. OCE cell-of-origin signature in a second ovarian tumor dataset (Tothill et al.) (Clin Cancer Res 2008; 14: 5198-208) which included mostly serous cancers (n=246) with a small subgroup of endometrioid tumors (n=20). In the Tothill dataset we observed a left skewing in the signature expression scores, which is consistent with a small subgroup of OV-like tumors. The tumors in the Tothill dataset were not microdissected, potentially causing a low signal to noise ratio due to stromal gene expression, and included just serous and endometrioid tumor subtypes. Thus, the lack of distinct bimodality of the scores in the Tothill dataset is likely due to these factors. In contrast, the tumor samples in the Wu dataset represented a diverse distribution of histological subtypes and were purified with microdis section, thus allowing a more direct comparison of the patient tumor signature with a signature derived from cultured epithelial cells without the interference of stromal signals.

Nonetheless, in the Tothill dataset, the FT-like subgroup was significantly enriched for serous tumors (P<0.01) and contained more advanced stage tumors (P=0.07) (FIG. 7b). However, no association with tumor grade was found (P=0.87) possibly because the Tothill data set included few low grade lesions. We also evaluated the associations with tumor histological subtype, grade and stage in the Wu and Tothill datasets using the continuous score from the cell-of-origin signature and observed similar results to those based on the FT/OV-like bipartition.

Analyses of patient survival in the Tothill dataset demonstrated that FT-like tumors had significantly worse disease-free survival (univariate log-rank P<0.001) and overall survival (univariate P=0.0495) (FIG. 7c). In multivariate analysis, after adjusting for tumor grade, stage, serous subtype, patient age and residual disease, the OV/FT-like subgroups were associated with disease-free survival (Cox proportional hazards P=0.01), but not overall survival (P=0.34).

Most ovarian cancer gene expression datasets are composed of high grade serous tumors that are thought to arise in the fallopian tube. These datasets underrepresent borderline and low grade tumors, as well as various histological subtypes such as endometrioid, clear cell, mucinous and transitional ovarian cancers. For example, the TCGA dataset (Nature 2011; 474: 609-15) includes only serous high grade tumors (n=491). In this dataset 6/10 probesets were available for analysis due to platform differences. With these 6 probesets we found that the FNE vs. OCE signature classified 43 TCGA tumors as OV-like and 448 tumors as FT-like. Hence, perhaps not surprisingly, there was little variability in the signature scores and there was no association of these subgroups with patient survival in the TCGA dataset. In the Tothill dataset, 20 high grade serous tumors were classified as OV-like and 217 tumors as FT-like. These results are consistent with the notion that most high grade serous carcinomas may indeed arise in the fallopian tube, but also highlight the limitations of these datasets in order for evaluating the FNE vs. OCE cell-of-origin signature.

To directly test the influence of the normal cell-of-origin on the associated tumor phenotype, we also created transformed derivatives of hTERT immortalized FNE and OCE cells by sequential introduction of SV40 Large T/small t (SV40T/t) antigen and H-Ras as we described before (Ince et al., Cancer Cell 2007; 12: 160-170 and Hahn et al., Nature 1999; 400: 464-8); these tumorigenic cells are hereafter referred to as FNLER and OCLER, respectively. Equal numbers of transformed FNLER and OCLER cells were injected into the intraperitoneal space and subcutaneous sites of 24 immunodeficient nude (Nu/Nu) mice (12 mice per cell type). Necropsy analyses of mice after 5-9 weeks after injection revealed similar rates of xenograft formation, total tumor burden and tumor histopathology (poorly differentiated with focal micropapillary-like architecture) in both cell types. Both FNLER and OCLER derived tumors were highly invasive into the surrounding intraperitoneal tissues (FNLER invasion). Examination of the lungs from mice bearing tumors (>0.5 g) revealed striking differences in the propensity to develop lung metastases; FNLER formed metastases in the lungs of 67% of mice (n=6) while isogenic OCLER formed metastases in only 13% of the mice (n=8) (P=0.04, Mann-Whitney test). The number of metastatic cells in each set of lungs was also higher in mice bearing FNLER tumors. The presence of the metastatic cells were confirmed in FFPE mouse lungs with H&E and immunohistochemical staining for p53 and SV40. No difference was observed in the total tumor burden or the time of tumor incubation in mice that were examined for lung metastases. These in vivo data, combined with our previous observations that FT-like patient tumors were associated with worse outcome, suggests that the normal cell of origin may indeed play a role in determining the associated tumor phenotype.

### Supplementary Methods

Tissue collection. All study procedures were approved by the Internal Review Board to collect discarded tissues. The study protocol allowed limited access to clinical information to exclude women who had an increased genetic risk for ovarian cancer or those currently taking medications that could modify their ovaries or fallopian tubes. During the optimization period, we tested various medium formulations and cell collection methods over several years, which were tested on a total of 37 samples, including 18 tissue fragments that were collected at the pathology suite following surgery and 19 tissue scrapes that were collected in the operating room.

The tissue fragments collected following surgery were dissociated mechanically or enzymatically and plated in various formulations of WIT-fo medium. With this approach we were not able to establish any short term ovarian cells in culture, and only three fallopian tube cultures could be established. In contrast, collecting surface scrapings during surgery was more successful. In this approach the scrapings from the normal ovary and fallopian tube (fimbriated end) were collected during the surgery using an endoscopic kittner (e.g., Aspen Surgical) from patients undergoing surgery for benign gynecologic conditions. Among these patients we were able to establish cells in culture from the fallopian tube fimbria in approximately 75% of the cases, and approximately 30% from the ovarian surface epithelium. However, in many cases paired normal ovarian surface and fallopian tube epithelial cells from the same patient were not available, either because only one of the tissues could be sampled, or they were not both disease free or one of them was removed in a previous surgery. We were also using these samples to optimize WIT-fo medium formulations therefore even in cases where both tissues were collected, one of the cell pairs was sometimes lost due to growth arrest or cell death during the optimization period. However, once all conditions were optimized we were able to establish paired ovarian surface and fallopian tube epithelial cell lines from two patients who were 56 and 65 years old and did not have any type of gynecologic cancer. The ovaries and fallopian tubes were disease free.

Ovarian surface epithelium: The normal ovarian surface epithelium is very delicate such that even gentle handling during surgery immediately strips away most of the normal surface epithelium. In order to collect the surface lining of the ovary, the cells need to be collected before the organ is handled extensively by the surgeon or the pathologist during routine surgical procedures. The ovarian inclusion cyst epithelium is sometimes located directly adjacent to the ovarian surface with no cell layers in between, or may be separated from the surface by just a few stromal cells and on occasion the cysts open up to the surface focally. Hence, a firm scraping of the ovarian surface can detach inclusion cyst epithelium.

Fallopian tube fimbria epithelium: To establish paired ovarian surface epithelium and fallopian tube epithelium cultures, we collected specimens in the operating room before their removal from the patient. Fallopian tube epithelial cells were collected using an endoscopic kittner, by rolling the fimbria around the end of the kittner. Cells were immediately placed into the WIT-fo cell culture media and then transferred into a small tissue culture dish (e.g., 1 or 2 wells of a 6-well plate to maximize cell density) and placed in a tissue culture incubator as soon as possible. It has been easier to establish fallopian tube epithelial cultures from specimens that have been removed from patients, likely due to the abundance of epithelial cells in the fallopian tube fimbria compared to the ovarian surface epithelium.

Culture of primary normal human fallopian tube and ovarian epithelium. The cells that were collected from fallopian tube and ovary were immediately placed in WIT-fo cell culture media and transferred to a tissue culture flask with a modified surface treatment (Primaria, BD Biosciences, Bedford, Mass.) and incubated at 37° with 5% CO₂ in ambient air. Please note that we strongly recommend the use of these culture plates since in our experience it will be nearly impossible to grow these cells in regular tissue culture plastic ware. Incubating the cells in lower O₂ levels did not improve the results, nor were we able to establish long term cultures using regular tissue culture plastic. WIT-fo is a modified version of WIT medium that we previously described (Bast et al., Nat Rev Cancer 2009; 9: 415-28) (Stemgent, Cambridge, Mass.). In order to adapt WIT medium for ovarian and fallopian tube epithelial cells it was modified with several supplements to a final concentration of 0.5 to 1% serum. The normal human epithelial cells are normally not in direct contact with blood or serum under physiologic conditions. Thus, the medium we use for most normal cells is completely serum-free in order to mimic physiologic conditions. However, cells on the surface of normal ovary and the fimbriated end of the fallopian tubes are directly in contact with normal peritoneal fluid which contains physiologic serum proteins. Indeed, the concentration of these serum proteins can be as high as fifty percent of the circulating blood. Thus, we added serum into WIT medium in order to mimic the physiologic growth conditions of normal ovarian cells. In addition to low concentrations of serum (0.5-1%), the WIT medium was supplemented with EGF (0.01 ug/mL, Sigma, E9644), Insulin (20 ug/mL, Sigma, I0516), Hydrocortisone (0.5 ug/mL, Sigma H0888) and 25 ng/mL Cholera Toxin (Calbiochem, 227035) in order to prepare WIT-fo medium. After 10-15 days, during which the medium was changed every 2-3 days, cells were lifted from the tissue culture plastic ware using 0.05% trypsin at room temperature while continuously checking and tapping the tissue culture flask to dislodge cells and therefore minimize exposure to trypsin (˜15-30 seconds exposure to trypsin or longer times only if necessary). Trypsin was inactivated using medium containing 10% serum, followed by centrifugation of cells in polypropylene tubes (500×g, 4 minutes) to remove excess trypsin and serum. Subcultures were established by seeding cells at a minimum density of 1×10⁴/cm² (a split ratio of 1:2 was generally applied, i.e. one flask of cells was split and seeded into two equivalent-sized flasks). However we highly recommend counting cells to seed at the required minimum density rather than relying on a split ratio. Medium was replaced 24 hrs after re-plating cells and every 48-72 hours thereafter. Primary cell cultures were generally split every 1-2 weeks or when cells reached ˜90-95% density.

The normal ovarian surface and fallopian tube epithelial cells were cultured in WIT-fo medium beyond 10 population doublings, while replicate plates of the same cells cultured under standard media conditions stopped growing after a few passages. In many previous reports the success in culturing normal ovarian and fallopian tube epithelium has been described as the number of cell passages that was achieved. It is worth noting that cell passage number refers to the number of times the cells are successfully lifted from one plate and seeded into a new culture plate. This indicates that at least some of the cells can tolerate the transfer and are still alive. However, passage number does not necessarily correlate with proliferation and an associated net increase in the number of cells. For example, we were able to ‘passage’ fallopian tube epithelium for nearly 60 days, with 4 passages in control medium. However, the curve was almost flat after 14 days and there was no net increase in the number of cells.

A fair comparison with our results would be in terms of ‘population doublings’, or the log 2 of the number of cells harvested less the number of cells seeded; hence 2 cells expand to 1,024 cells in 10 population doublings (2¹⁰=1,024). Each 10 population doublings is approximately equal to 3 orders of magnitude (×10³) net increase in cell numbers, 20 population doublings would be close to a 1 million fold increase and 30 population doublings would be close to a 1 billion fold increase in net cell numbers. In contrast, cell passages may be equal to almost no net increase in cell numbers. For example, ovarian epithelial cells grown in WIT-fo medium reach 14 population doublings in 7 passages (42 days), an 8,192-fold increase in net cell number. In standard control medium the same cells could be passaged 7 times (42 days) as well, however, they only had 2.4 population doublings which is equal to a 5.3-fold net increase in cell numbers, thus the same cells increased in number 1,546 times more in WIT-fo than in standard medium (8,192÷5.3=1,526).

To culture ovarian epithelial cells, we tested several previously described cell culture media (Ince et al., Cancer Cell 2007; 12: 160-170; Visvader et al., Nature 2011; 469: 314-22; Dubeau, Lancet, Oncol 2008; 9: 1191-7), including a 1:1 mixture of MCDB 105/Medium 199 with a range of 5-10% fetal bovine serum, 2 mM 1-glutamine with and without 10 ng/ml epidermal growth factor, and Dulbecco's modified Eagle's medium (DMEM)/Ham's F-12 (1:1 mixture) with 10-15% fetal bovine serum. In neither case were we able to propagate ovarian epithelial cells beyond a few population doublings. The ovarian epithelial cell growth rates that we observed when using the MCDB 105/Medium 199/10% fetal bovine serum control medium (˜2 population doublings) were within the lower range (2-12 population doublings using MCDB 105/Medium 199/15% fetal bovine serum) previously reported by Auersperg et al. (J Cell Physiol 1997; 173: 261-5 and Lab Invest 1994; 71: 510-8).

For fallopian tube epithelium culture we tested several previously described media (Piek et al., J Pathol 2001; 195: 451-6; Lee et al., J Pathol 2007; 211: 26-35; Kindelburger et al., Am J Surg Pathol 2007; 31: 161-9; and The Cancer Genome Atlas Research Network Nature 2011; 474: 609-15), a 1:1 mixture of DMEM/Ham's F12, supplemented with 0.1% BSA, 5% serum (1:1 mix of 2.5% fetal bovine serum plus 2.5% Nu Serum) and 17β estradiol, or a slightly modified version of this medium supplemented with 2% serum substitute. None of the above-mentioned traditional cell culture media that we tested supported long-term propagation of normal epithelial cells from human ovary or fallopian tube. Additional notes on culturing primary normal human fallopian tube and ovarian epithelial cells include:
Maintain the cells in large media volumes, which are greater than typical volumes;
   e.g. T25 flask=10 mls; T75 flask=28 mls
   6 cm plate=4 mls; 10 cm plate=15 mls
Change media every 2-3 days, or sooner if the media turns a yellowish/brown color.
Trypsinization: Cells trypsinize quickly (<1 min when adding trypsin at room temperature); inactivate trypsin as soon as cells come off the flask, otherwise cells will not survive. Trypsinize using freshly defrosted 0.05% trypsin, followed by trypsin inactivation in 10-20% serum containing media (aliquot trypsin & freeze for this purpose). Alternatively, ‘Tryple Express’ (BD) for trypsinization (designed for serum-free cell cultures) can be used to detach the cells from the plate (following the manufacturer's instructions).
Cell seeding density: Minimum seeding density ≧10,000 cells per cm² of growth area (tissue culture plate), e.g. seed 400,000 cells into one T25 flask.
Freezing cells: ‘Bambanker’ freeze down media works well (Bambanker, produced by Lymphotec Inc, is distributed by Wako Laboratory Chemicals) (follow manufacturer's instructions for use). Cells can also be frozen in 10% DMSO in media containing 20% serum, but this method is not as optimal as Bambanker. Freeze cells in a “Mr Freeze” container (Nalgene) (as per manufacturer's instructions).

Retroviral infections. Amphotropic retroviruses (for pmig-GFP-hTERT) were produced by transfection of the 293T producer cell line with 1 μg of retroviral vector and 1 μg total of the packaging (pUMVC3) and envelope (pCMV-VSV-G) plasmids at an 8:1 ratio using Fugene 6 (Roche Applied Science, Indianapolis, Ind.). Viral supernatants were harvested at 24 and 48 hrs and used to infect primary ovarian surface and fallopian tube epithelial cells with 8 μg/ml polybrene. Retroviruses were sequentially introduced to recipient cells in individual steps in the following order: pmig-GFP-hTERT, pBABE-zeo-SV40-ER and pBABE-puro-H-ras V12. Selection of infected cells was performed serially and drug selection (500 μg/ml zeocin (zeo) and 1 μg/ml puromycin (puro)) was used to purify polyclonal infected populations after each infection. Primary ovarian surface epithelial cells were immortalized with hTERT between passages 2 to 6 and transformed between passages 26 to 30. Primary fallopian tube epithelial cells were transduced with hTERT between passages 1 to 4 and transformed at passage 16. Cells immortalized with hTERT and those that were transduced with SV40 and/or H-ras were cultured in WIT-fo media on Primaria tissue culture ware (BD Biosciences). All protocols involving the use of retroviruses were approved by the Committee on Microbiological Safety.

Immortalized ovarian surface and fallopian tube epithelial cell lines (containing only the pmig-GFP-hTERT vector) will are referred to as OCE and FNE and fully transformed derivatives as OCLER and FNLER following the introduction of vectors encoding hTERT (E), SV40 early region (L) and HRas (R).

Analysis of tumorigenicity and metastasis. The protocol for tumorigenesis experiments in immunocompromised mice was approved by the Harvard Standing Committee on Animals. All experiments were performed in compliance with relevant institutional and national guidelines and regulations. Single-cell suspensions were prepared in a Matrigel: WIT-fo mixture (1:1) and 1 million cells per 100 μl volume were injected in one intraperitoneal (IP) and two subcutaneous (SC) sites per mouse. Tumor cell injections were performed on 6-8 week old female immunodeficient nude (Nu/Nu) mice (Charles River Laboratories International, Inc, Wilmington, Mass.). Tumors were harvested 5 to 9 weeks after implantation of tumorigenic cells from tissue culture into IP and SC sites in nude mice. Tumor histopathology was assessed from hematoxylin and eosin stained sections from formalin-fixed paraffin-embedded (FFPE) tissues. Immunohistochemistry was carried out on FFPE tissues using cell type specific markers (CK7, FOXJ1, PAX8) to determine immunostaining patterns in mouse OCLER and FTLER xenografts as well as normal human ovaries and fallopian tubes (discarded tissues collected under an IRB-approved protocol). Metastasis of GFP-expressing tumor cells to lungs was analyzed initially using an Olympus SZX16 Stereo Fluorescence microscope in fresh tissues, followed by microscopic examination of hematoxylin and eosin stained sections of FFPE tissues. The presence of tumor cells in mouse lungs was confirmed by immunostaining for SV40 LT (v-300) and p53 (FL-393) (Santa Cruz Biotechnology, Santa Cruz, Calif.). Immunostaining was carried out using the conventional ABC technique.

Live cell imaging and fluorescence activated cell sorting. Cells were grown for two days on untreated fluorodishes (World Precision Instruments, Sarasota, Fla.) and images of live cells were taken at 40× magnification with oil immersion using the Nikon TE2000-U inverted microscope and EZ-C1 software (Nikon) for image acquisition. Fluorescence activated cell sorting (FACS) analysis using a FACS Aria multicolor high speed sorter (BD Biosciences, San Jose, Calif.) was applied to quantify the proportion of ovarian and fallopian tube cells that were GFP positive following infection with pmig-GFP-hTERT.

Microarray data normalization and analysis. Affymetrix microarrays of hTERT immortalized cell lines (OCE, FNE) and publically available ovarian cancer datasets by Wu et al. (Cancer Cell 2007; 11: 321-33) (GEO Series accession number GSE6008) and Tothill et al. (Clin Cancer Res 2008; 14: 5198-208) (GEO Series accession number GSE9891) were independently normalized using the variance stabilization method (vsnrma) in R. We also used the TCGA mRNA expression data that was normalized by the TCGA consortium (Nature 2011; 474: 609-15). Comparisons of gene expression between cell lines were performed using 12 Human Genome U133 Plus 2 microarrays (HG U133Plus2.0, Affymetrix, Santa Clara, Calif.) measuring 54,675 probes. Samples that were arrayed included two biological replicates (paired hTERT immortalized OCE and FNE cells from two patients) and three experimental replicates (different passages) from each cell line. Microarray CEL files are available at GEO (GSE37648).

We first applied complete linkage hierarchical clustering (euclidean distance) based on global gene expression profiles and observed the strongest separation by patient (1 or 2) and the next subdivision of samples was by cell type (ovary or fallopian tube). To identify genes that were differentially expressed between epithelial cells of fallopian tube vs ovarian origin, we applied a modified t-test (P<0.05) using Linear Models for Microarray Data (Smyth et al., Stat Appl Genet Mol Biol 2004; 3: Article3) (Limma) and corrected for the False Discovery Rate (FDR). Setting the FDR adjusted P-value cutoff <0.05, 1,157 probesets varied significantly between immortalized (FNE vs OCE) cells. Since we observed differences between patients in unsupervised hierarchical clustering analysis, we applied the duplicate correlation function in Limma (Auersperg et al., Lab Invest 1994; 71: 510-8) to identify differentially expressed genes between FNE and OCE while blocking for patient differences.

To classify human ovarian tumors as fallopian tube (FT)-like and ovary (OV)-like within three publically available ovarian cancer datasets (detailed above), we sorted the FNE vs OCE genelist based on FDR-adjusted P-values and selected ten probesets with unique gene symbols that were over-expressed in either FNE or OCE and calculated the sum of the normalized expression values of these ten probesets in two ovarian cancer datasets by weighting FNE probesets by (+1) and OCE probesets by (−1); specifically, the sum of the normalized expression values of OCE genes were subtracted from the sum of expression values of FNE genes to calculate a score for each tumor (e.g. a higher score tumor is more FT-like). We then fit a bimodal distribution of Gaussian curves using mixture modeling to this score to identify two groups of tumors within each dataset, those that were more OV-like or FT-like.

We first performed this clustering in the Wu et al. (Cancer Cell 2007; 11: 321-33) dataset that contains expression profiles of 99 fresh frozen, microdissected epithelial ovarian cancers (including many non-serous histologic subtypes) arrayed on a similar platform (Affymetrix HG U133A). Eight of the 10 selected probesets were available for analysis due to array platform differences. We used this cell-of-origin signature to define FT-like and OV-like subpopulations in the Wu data (as discussed above) and visualized the distribution of these scores using density plots to determine the validity of this classification. We evaluated the clinical characteristics of patient tumors classified as FT-like/OV-like and calculated their associated P-values using ordinal logistic regression (grade, stage) or Fisher's Exact Test (histologic subtype).

The cell-of-origin signature was further validated in the Tothill (Clin Cancer Res 2008; 14: 5198-208) dataset which includes 246 serous and 20 endometrioid fresh frozen malignant tumors (not microdissected) that were arrayed on the HG U133 Plus 2.0 platform (Affymetrix) and importantly in this dataset gene expression patterns can be linked with patient survival data. Similar methods for Gaussian mixture modeling and tumor classification as described above were applied to the Tothill dataset. To assess whether the FT-like/OV-like classification was associated with differences in patient disease-free and overall survival, we constructed Kaplan-Meier plots and calculated univariate P-values using the log-rank test. We then applied a Cox proportional hazards test, adjusting for grade, stage, serous histologic subtype, patient age and residual disease, to determine multivariate statistical significance.

Lastly, using the same methods described above we tested the FNE vs. OCE cell-of-origin signature in the TCGA dataset (Nature 2011; 474: 609-15), which includes 491 serous high grade tumors (tumors that were missing stage/grade were excluded). In the TCGA dataset 6/10 probesets were available due to platform differences. All microarray and survival analyses were conducted using R version 2.10.1.

Mesothelial versus Mullerian phenotypes of ovarian epithelium. The ovarian surface epithelium is in general very similar to the flat or cuboidal cells of the mesothelium that lines the peritoneal surfaces, and has a predominantly mesothelial-like morphology. A second subpopulation of ovarian epithelial cells with columnar and/or ciliated epithelium that is consistent with a Mullerian phenotype can be occasionally identified on the ovarian surface. The ovarian inclusion cyst epithelium is traditionally thought to arise from an invagination of the ovarian surface epithelium into the underlying stroma and both mesothelial and Mullerian phenotypes have been observed in the ovarian inclusion cyst epithelium. The ovarian epithelial (OCE) cells that we cultured exhibited a Mullerian phenotype among these cell types. Ovarian epithelial cells with a Mullerian phenotype in normal adult ovaries may result from exposure of the ovarian epithelium to the microenvironment or hormonal milieu in the ovarian cortex or may originate in the uterus or fallopian tube, and implant themselves onto the ovarian surface by the retrograde flow of the endometrial cells, exfoliation or direct contact via tubal adhesions. We predominantly observed Mullerian phenotype ovarian epithelium only in the ovarian inclusion cysts, thus favoring that OCE cells may originate in Mullerian phenotype ovarian inclusion cyst epithelium. However, a recent study by Li et al. (Mod Pathol 2011) described rare Mullerian phenotype cells on the ovarian surface in addition to the inclusion cysts. Hence the cultured OCE cells also may have originated from Mullerian phenotype epithelial cells on the ovarian surface in addition to the cyst epithelium.

In summary, we demonstrated that the cell-of-origin may mediate important differences in ovarian tumor phenotype. In light of other findings that suggest that the same oncogenes can have vastly different phenotypic consequences depending on the cell-of-origin, an approach that combines the ongoing efforts to survey the genetic mutational spectrum in various types of tumors with contextual information about the cell-of-origin and differentiation state of each tumor is needed to evaluate the effects of different genetic aberrations. In ovarian cancer, this information may assist to devise approaches for personalized medicine based on the cell-of-origin classification and to address the role of site-of-origin in cancer prevention models. Here we describe a new culture system that will greatly improve our ability to study the role played by different cells-of-origin in the pathogenesis of ovarian carcinomas.

### Example 4

### Panel of Oncology Drugs

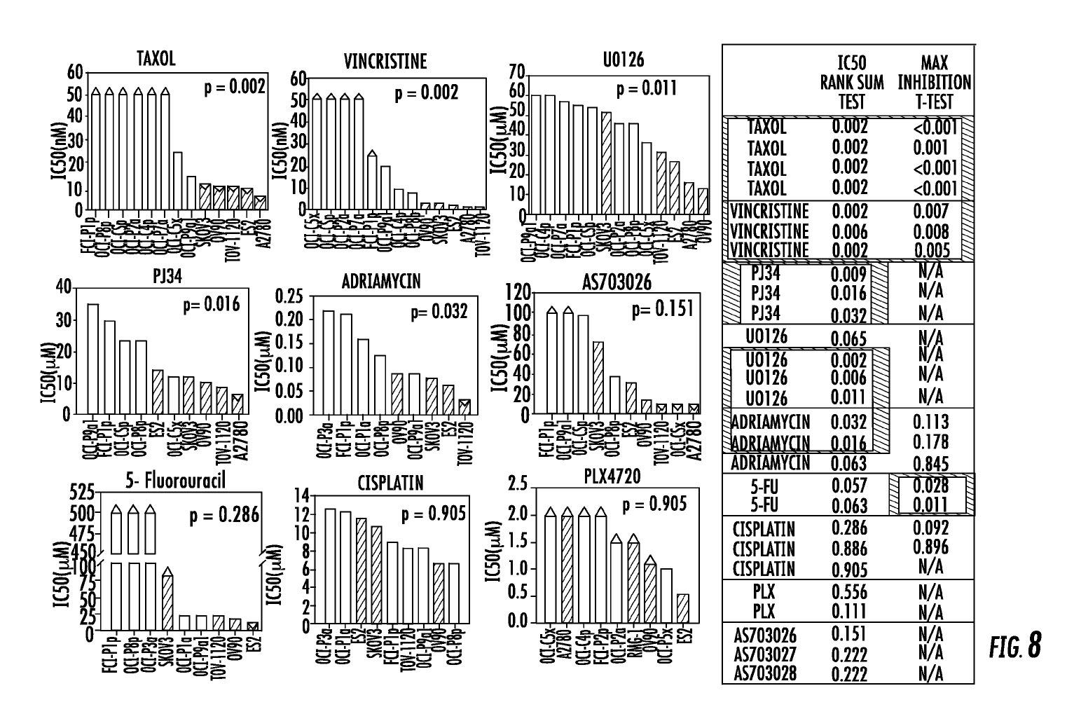

Referring to FIG. 8, our results show that OCI lines are significantly more resistant to a diverse panel of oncology drugs compared to standard cell lines. In these experiments, ATCC ovarian tumor lines (SKOV3, OV90, TOV-1120 and A2780) and OCI ovarian tumor lines (FCI-P2p, OCI-P5x, OCI-P2a, OCI-C4p, OCI-P7a, OCI-05x, OCI-CSp, FCI-P1p, OCI-P9a1, OCI-P8p, OCI-P3a, OCI-P1a) were plated in WIT-OC medium (5000 cells/well) in 96 well plates. The next day serial dilutions of Taxol, Vincristine, U0126, PJ34, Adriamycin, AS703026, 5-fluorouracil, Cisplatin, PLX4720 and PJ34 were added. The number of viable cells was measured as 590/530 florescence via Alamar Blue after 72-144 hrs incubation, depending on drug. We found that the OCI lines were in general more resistant to inhibition of cell proliferation by Poly (ADP-ribose) polymerase (PARP) inhibitor PJ34, which may be consistent with the low level of response seen to this drug in the clinic so far. OCI lines were also more resistant to MAPK inhibitor U0126 and DNA intercalating drug Adriamycin and 5-fluorouracil. Thus, the methods described herein of analyzing sensitivity of a subject's cancerous tumor to an oncology drug and developing a personalized therapy for the subject can be used for any drug.

### Other Embodiments

Any improvement may be made in part or all of the assays, kits, and method steps. All references, including publications, patent applications, and patents, cited herein are hereby incorporated by reference. The use of any and all examples, or exemplary language (e.g., “such as”) provided herein, is intended to illuminate the invention and does not pose a limitation on the scope of the invention unless otherwise claimed. Any statement herein as to the nature or benefits of the invention or of the preferred embodiments is not intended to be limiting, and the appended claims should not be deemed to be limited by such statements. More generally, no language in the specification should be construed as indicating any non-claimed element as being essential to the practice of the invention. Although the experiments described herein pertain to ovarian cancer, the assays, method and kits described herein can be applied to any cancer. This invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contraindicated by context.

What is claimed is:

1. A method for analyzing sensitivity of a subject's cancerous tumor to an oncology drug and developing a personalized therapy for the subject, the method comprising the steps of:
(a) obtaining cancer cells from the subject's cancerous tumor;
(b) examining expression of a set of proteins or mRNAs in the cancerous cells, wherein overexpression or underexpression of the set of proteins or mRNAs relative to a control is associated with resistance to the oncology drug; and
(c) correlating overexpression or underexpression of the set of proteins or mRNAs relative to the control with resistance of the subject's cancerous tumor to the oncology drug and correlating normal expression of the set of proteins or mRNAs relative to the control with sensitivity of the subject's cancerous tumor to the oncology drug.

2. The method of claim 1, wherein the oncology drug is selected from the group consisting of: Taxol, vincristine, U0126, PJ34, adriamycin, AS703026, 5-Fluorouracil, cisplatin, and PLX4720.

3. The method of claim 1, wherein the set of proteins or mRNAs are overexpressed or underexpressed in the subject's cancerous tumor relative to the control, and the method further comprises administering to the subject an oncology drug different from the oncology drug the subject's cancerous tumor is resistant to.

4. The method of claim 3, wherein the different oncology drug is selected from the group consisting of: Taxol, vincristine, U0126, PJ34, adriamycin, AS703026, 5-Fluorouracil, cisplatin, and PLX4720.

5. The method of claim 1, wherein the set of proteins or mRNAs are normally expressed relative to the control, and the method further comprises administering the oncology drug to the subject.

6. The method of claim 1, wherein the oncology drug is Taxol or vincristine, and the set of proteins comprises at least two proteins selected from the group consisting of: tubulin, AKT, androgen receptor, Jun oncogene, Crystalline, cyclin D1, epidermal fatty acid binding protein, Ets related gene, FAK, Forkhead Box O3, Erk/Mek, N-cadherin, mitogen-activated protein kinase 14, plasminogen activator inhibitor type 1, paired box 2, protein kinase C-alpha, protein kinase AMP-activated Gamma 2, phosphatase and tensin homolog, SMAD3, Sarcoma viral oncogene homolog, signal transducer and activator of transcription 3, and signal transducer and activator of transcription 5.

7. The method of claim 1, further comprising correlating overexpression or underexpression of the set of proteins or mRNAs relative to the control with a worse prognosis for the subject compared to a second subject having a cancerous tumor in which the first set of proteins or mRNAs are normally expressed relative to the control.

8. The method of claim 1, wherein the subject is a female human having an ovarian cancer tumor.

9. The method of claim 1, further comprising repeating steps b) and c) until an oncology drug that the subject's cancerous tumor is sensitive to is identified.

10. A method for predicting a response of a cancer patient's cancerous tumor to an oncology drug and developing a personalized therapy for the patient for treatment of the cancerous tumor, the method comprising the steps of:
a) obtaining cancer cells from the patient's cancerous tumor;
(b) culturing the cancer cells in WIT-OC, WIT-L, or WIT-OCe cell culture medium;
(c) contacting the cultured cancer cells with the oncology drug;
(d) determining an IC50 OR IC90 value for the oncology drug in the cultured cancer cells; and
(e) correlating an increased IC50 or IC90 value relative to an IC50 or IC90 value for the oncology drug in control cultured cells with a poor response of the patient's cancerous tumor to the oncology drug and correlating a normal or low IC50 or IC90 value relative to the IC50 or IC90 value for the oncology drug in control cultured cells with a positive response of the patient's cancerous tumor to the oncology drug.

11. The method of claim 10, wherein the cancer cells are ovarian cancer cells obtained from ascites fluid or primary solid ovarian tissue from the patient.

12. The method of claim 10, wherein the oncology drug is selected from the group consisting of: Taxol, vincristine, U0126, PJ34, adriamycin, AS703026, 5-Fluorouracil, cisplatin, and PLX4720.

13. The method of claim 10, wherein the IC50 or IC90 value is increased relative to the IC50 or IC90 value for the oncology drug in control cultured cells, and the method further comprises administering to the patient a second oncology drug.

14. The method of claim 13, wherein the second oncology drug is selected from the group consisting of: Taxol, vincristine, U0126, PJ34, adriamycin, AS703026, 5-Fluorouracil, cisplatin, and PLX4720.

15. The method of claim 10, wherein the IC50 or IC90 value is normal or decreased relative to the IC50 or IC90 value for the oncology drug in control cultured cells, and the method further comprises administering the oncology drug to the patient.

16. The method of claim 10, further comprising correlating an increased IC50 or IC90 value relative to an IC50 or IC90 value for the oncology drug in control cultured cells with a worse prognosis for the patient compared to a second patient having a cancerous tumor in which an IC50 or IC90 value for the oncology drug in cultured cancer cells from the second patient is normal or decreased relative to the IC50 or IC90 value for the oncology drug in control cultured cells.

17. The method of claim 10, wherein the patient is a female human having an ovarian cancer tumor.

18. A kit for analyzing sensitivity of a subject's cancerous tumor and predicting a response of a subject's cancerous tumor to an oncology drug and developing a personalized therapy for the subject, the kit comprising:
(a) one or more OCI lines as an internal control(s);
(b) instructions for use;
(c) WIT medium, or a derivative of WIT medium; and optionally,
(d) one or more probes.

19. The kit of claim 18, wherein the one or more probes comprise at least two probes specific to at least two proteins selected from the group consisting of: tubulin, AKT, androgen receptor, Jun oncogene, Crystalline, cyclin D1, epidermal fatty acid binding protein, Ets related gene, FAK, Forkhead Box O3, Erk/Mek, N-cadherin, mitogen-activated protein kinase 14, plasminogen activator inhibitor type 1, paired box 2, protein kinase C-alpha, protein kinase AMP-activated Gamma 2, phosphatase and tensin homolog, SMAD3, Sarcoma viral oncogene homolog, signal transducer and activator of transcription 3, and signal transducer and activator of transcription 5.

20. A method for determining a prognosis of a subject having an ovarian cancer tumor, the method comprising the steps of:
a) obtaining a sample from the subject's tumor;
b) subjecting the sample to gene expression profiling resulting in an expression profile comprising a first set of genes that are upregulated in fallopian tube cells relative to ovarian cells and a second set of genes that are upregulated in ovarian cells relative to fallopian tube cells;
c) determining expression levels of the first and second sets of genes; and
d) correlating an upregulation of the first set of genes but not of the second set of genes with a worse disease-free survival prognosis relative to a second subject having an ovarian cancer tumor in which the first set of genes are not upregulated and the second set of genes are upregulated.

21. The method of claim 20, wherein the first set of genes comprises DOK5, CD47, HS6ST3, DPP6, and OSBPL3 and the second set of genes comprises STC2, SFRP1, SLC35F3, SHMT2, and TMEM164.

22. The method of claim 20, wherein the first set of genes in the expression profile is upregulated, and the method further includes classifying the subject's ovarian cancer tumor as fallopian tube-like.

23. The method of claim 20, wherein the second set of genes in the expression profile is upregulated, and the method further includes classifying the subject's ovarian cancer tumor as ovary-like.

24. The method of claim 20, wherein the subject is a female human.

25. The method of claim 20, wherein the method further comprises administering an oncology drug to the subject.
